SEQUENCE LISTING (30 sequences)

SEQ ID NO: 1 - DNA, 4459 bp, synthetic construct
  misc_feature 1..4459: note = codon-optimised CFTR transgene (soCFTR2)
  source 1..4459: mol_type = other DNA
gctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggccc

SEQ ID NO: 2 - DNA, 574 bp, synthetic construct
  misc_feature 1..574: note = hCEF promoter
  source 1..574: mol_type = other DNA
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 3 - DNA, 873 bp, Cytomegalovirus
  source 1..873: mol_type = unassigned DNA
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 4 - DNA, 395 bp, Homo sapiens
  source 1..395: mol_type = unassigned DNA
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 5 - DNA, 4391 bp, Simian immunodeficiency virus
  source 1..4391: mol_type = unassigned DNA
atgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaa

SEQ ID NO: 6 - DNA, 4391 bp, synthetic construct
  misc_feature 1..4391: note = codon-optimised SIV gal-pol nucleic acid sequence
  source 1..4391: mol_type = other DNA
atgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattga

SEQ ID NO: 7 - DNA, 9064 bp, synthetic construct
  misc_feature 1..9064: note = pDNA2a pGM691
  source 1..9064: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccaccatgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattgaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 8 - DNA, 10528 bp, synthetic construct
  misc_feature 1..10528: note = pDNA1 pGM326
  source 1..10528: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 9 - DNA, 10536 bp, synthetic construct
  misc_feature 1..10536: note = pDNA1 pGM830
  source 1..10536: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaattgggggcggctacctcagcactaaataggagacaattagaccaatttgagaaaatacgacttcgcccgaacggaaagaaaaagtaccaaattaaacatttaatattgggcaggcaaggagattggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaattgcctgggtacatgtacccttgtcaccgcgcaccttaaatgcgtgggtaaaagcagtagaggagaaaaaatttggagcagaaatagtacccatgtttcaagccctatcgcctgcaggccgtttgtgctagggttcttaggcttcttgggggctgctggaactgcattgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaataagacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaaagggatttttagtaatagtaggaataatagggttaagattactttacacagtatatggatgtatagtgagggttaggcagggatatgttcctctatctccacagatccatataaagcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 10 - DNA, 9886 bp, synthetic construct
  misc_feature 1..9886: note = pDNA2a pGM297
  source 1..9886: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagactagtgacttggtgagtaggcttcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaaatggcagggaatagtcagatattggatgagacaaagaaatttgaaatggaactattatatgcatcagctggcggccgcgaattcactagtgattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccaatcgaattcccgcggccgcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 11 - DNA, 3384 bp, synthetic construct
  misc_feature 1..3384: note = pDNA2b pGM299
  source 1..3384: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgattatgcccctaggaccagaagaaagaagattgcttcgcttgatttggctcctttacagcaccaatccatatccaccaagtggggaagggacggccagacaacgccgacgagccaggagaaggtggagacaacagcaggatcaaattagagtcttggtagaaagactccaagagcaggtgtatgcagttgaccgcctggctgacgaggctcaacacttggctatacaacagttgcctgaccctcctcattcagcttagaatcactagtgaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgtcgaccaattgttgtgtctcaaaatctctgatgttacattgcacaagataaaaatatatcatcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctgttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 12 - DNA, 6264 bp, synthetic construct
  misc_feature 1..6264: note = pDNA3a pGM301
  source 1..6264: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgattgccatggcaacatatatccagagagtacagtgcatctcaacatcactactggttgttctcaccacattggtctcgtgtcagattcccagggataggctctctaacataggggtcatagtcgatgaagggaaatcactgaagatagctggatcccacgaatcgaggtacatagtactgagtctagttccgggggtagactttgagaatgggtgcggaacagcccaggttatccagtacaagagcctactgaacaggctgttaatcccattgagggatgccttagatcttcaggaggctctgataactgtcaccaatgatacgacacaaaatgccggtgctccccagtcgagattcttcggtgctgtgattggtactatcgcacttggagtggcgacatcagcacaaatcaccgcagggattgcactagccgaagcgagggaggccaaaagagacatagcgctcatcaaagaatcgatgacaaaaacacacaagtctatagaactgctgcaaaacgctgtgggggaacaaattcttgctctaaagacactccaggatttcgtgaatgatgagatcaaacccgcaataagcgaattaggctgtgagactgctgccttaagactgggtataaaattgacacagcattactccgagctgttaactgcgttcggctcgaatttcggaaccatcggagagaagagcctcacgctgcaggcgctgtcttcactttactctgctaacattactgagattatgaccacaatcaggacagggcagtctaacatctatgatgtcatttatacagaacagatcaaaggaacggtgatagatgtggatctagagagatacatggtcaccctgtctgtgaagatccctattctttctgaagtcccaggtgtgctcatacacaaggcatcatctatttcttacaacatagacggggaggaatggtatgtgactgtccccagccatatactcagtcgtgcttctttcttagggggtgcagacataaccgattgtgttgagtccagattgacctatatatgccccagggatcccgcacaactgatacctgacagccagcaaaagtgtatcctgggggacacaacaaggtgtcctgtcacaaaagttgtggacagccttatccccaagtttgcttttgtgaatgggggcgttgttgctaactgcatagcatccacatgtacctgcgggacaggccgaagaccaatcagtcaggatcgctctaaaggtgtagtattcctaacccatgacaactgtggtcttataggtgtcaatggggtagaattgtatgctaaccggagagggcacgatgccacttggggggtccagaacttgacagtcggtcctgcaattgctatcagacccgttgatatttctctcaaccttgctgatgctacgaatttcttgcaagactctaaggctgagcttgagaaagcacggaaaatcctctcggaggtaggtagatggtacaactcaagagagactgtgattacgatcatagtagttatggtcgtaatattggtggtcattatagtgatcatcatcgtgctttatagactcagaaggtgaaatcactagtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 13 - DNA, 6522 bp, synthetic construct
  misc_feature 1..6522: note = pDNA3b pGM303
  source 1..6522: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagcatgtggtctgagttaaaaatcaggagcaacgacggaggtgaaggaccagaggacgccaacgacccccggggaaagggggtgcaacacatccatatccagccatctctacctgtttatggacagagggttagggatggtgataggggcaaacgtgactcgtactggtctacttctcctagtggtagcaccacaaaaccagcatcaggttgggagaggtcaagtaaagccgacacatggttgctgattctctcattcacccagtgggctttgtcaattgccacagtgatcatctgtatcataatttctgctagacaagggtatagtatgaaagagtactcaatgactgtagaggcattgaacatgagcagcagggaggtgaaagagtcacttaccagtctaataaggcaagaggttatagcaagggctgtcaacattcagagctctgtgcaaaccggaatcccagtcttgttgaacaaaaacagcagggatgtcatccagatgattgataagtcgtgcagcagacaagagctcactcagcactgtgagagtacgatcgcagtccaccatgccgatggaattgccccacttgagccacatagtttctggagatgccctgtcggagaaccgtatcttagctcagatcctgaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtggcaaccgggactaggggttatcagctttgctccatgccgactgtagacgaaagaaccgactactctagtgatggtattgaggatctggtccttgatgtcctggatctcaaagggagaactaagtctcaccggtatcgcaacagcgaggtagatcttgatcacccgttctctgcactataccccagtgtaggcaacggcattgcaacagaaggctcattgatatttcttgggtatggtggactaaccacccctctgcagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaataaagagtgcaattggtacaataagtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaagcggccgcgcatgcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtctattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 14 - DNA, 600 bp, Woodchuck hepatitis B virus
  source 1..600: mol_type = unassigned DNA
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct

SEQ ID NO: 15 - DNA, 1738 bp, synthetic construct
  misc_feature 1..1738: note = CAG promoter
  source 1..1738: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccacc

SEQ ID NO: 16 - RNA, 7553 nt, synthetic construct
  source 1..7553: mol_type = other RNA
tctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaattgggggcggctacctcagcactaaataggagacaattagaccaatttgagaaaatacgacttcgcccgaacggaaagaaaaagtaccaaattaaacatttaatattgggcaggcaaggagattggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaattgcctgggtacatgtacccttgtcaccgcgcaccttaaatgcgtgggtaaaagcagtagaggagaaaaaatttggagcagaaatagtacccatgtttcaagccctatcgcctgcaggccgtttgtgctagggttcttaggcttcttgggggctgctggaactgcattgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaataagacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaaagggatttttagtaatagtaggaataatagggttaagattactttacacagtatatggatgtatagtgagggttaggcagggatatgttcctctatctccacagatccatataaagcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagct

SEQ ID NO: 17 - protein, 502 aa, synthetic construct
QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPVDISLNLADATNFLQDSKAELEKARKILSEVGRWYNSRETVITIIVVMVVILVVIIVIIIVLYRLRR

SEQ ID NO: 18 - protein, 527 aa, synthetic construct
MATYIQRVQCISTSLLVVLTTLVSCQIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPVDISLNLADATNFLQDSKAELEKARKILSEVGRWYNSRETVITIIVVMVVILVVIIVIIIVLYRLRR

SEQ ID NO: 19 - protein, 411 aa, synthetic construct
FFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPVDISLNLADATNFLQDSKAELEKARKILSEVGRWYNSRETVITIIVVMVVILVVIIVIIIVLYRLRR

SEQ ID NO: 20 - protein, 91 aa, synthetic construct
QIPRDRLSNIGVIVDEGKSLKIAGSHESRYIVLSLVPGVDFENGCGTAQVIQYKSLLNRLLIPLRDALDLQEALITVTNDTTQNAGAPQSR

SEQ ID NO: 21 - protein, 25 aa, synthetic construct
MATYIQRVQCISTSLLVVLTTLVSC

SEQ ID NO: 22 - protein, 140 aa, synthetic construct
GAATSALNRRQLDQFEKIRLRPNGKKKYQIKHLIWAGKEMERFGLHERLLETEEGCKRIIEVLYPLEPTGSEGLKSLFNLVCVLYCLHKEQKVKDTEEAVATVRQHCHLVEKEKSATETSSGQKKNDKGIAAPPGGSQNF

SEQ ID NO: 23 - protein, 231 aa, synthetic construct
PAQQQGNAWVHVPLSPRTLNAWVKAVEEKKFGAEIVPMFQALSEGCTPYDINQMLNVLGDHQGALQIVKEIINEEAAQWDVTHPLPAGPLPAGQLRDPRGSDIAGTTSSVQEQLEWIYTANPRVDVGAIYRRWIILGLQKCVKMYNPVSVLDIRQGPKEPFKDYVDRFYKAIRAEQASGEVKQWMTESLLIQNANPDCKVILKGLGMHPTLEEMLTACQGVGGPSYKAKVM

SEQ ID NO: 24 - protein, 54 aa, synthetic construct
VQQGGPKRQRPPLRCYNCGKFGHMQRQCPEPRKTKCLKCGKLGHLAKDCRGQVN

SEQ ID NO: 25 - protein, 101 aa, synthetic construct
FELPLWRRPIKTVYIEGVPIKALLDTGADDTIIKENDLQLSGPWRPKIIGGIGGGLNVKEYNDREVKIEDKILRGTILLGATPINIIGRNLLAPAGARLVM

SEQ ID NO: 26 - protein, 441 aa, synthetic construct
GQLSEKIPVTPVKLKEGARGPCVRQWPLSKEKIEALQEICSQLEQEGKISRVGGENAYNTPIFCIKKKDKSQWRMLVDFRELNKATQDFFEVQLGIPHPAGLRKMRQITVLDVGDAYYSIPLDPNFRKYTAFTIPTVNNQGPGIRYQFNCLPQGWKGSPTIFQNTAASILEEIKRNLPALTIVQYMDDLWVGSQENEHTHDKLVEQLRTKLQAWGLETPEKKVQKEPPYEWMGYKLWPHKWELSRIQLEEKDEWTVNDIQKLVGKLNWAAQLYPGLRTKNICKLIRGKKNLLELVTWTPEAEAEYAENAEILKTEQEGTYYKPGIPIRAAVQKLEGGQWSYQFKQEGQVLKVGKYTKQKNTHTNELRTLAGLVQKICKEALVIWGILPVLELPIEREVWEQWWADYWQVSWIPEWDFVSTPPLLKLWYTLTKEPIPKEDVY

SEQ ID NO: 27 - protein, 120 aa, synthetic construct
YVDGACNRNSKEGKAGYISQYGKQRVETLENTTNQQAELTAIKMALEDSGPNVNIVTDSQYAMGILTAQPTQSDSPLVEQIIALMIQKQQIYLQWVPAHKGIGGNEEIDKLVSKGIRRVL

SEQ ID NO: 28 - protein, 291 aa, synthetic construct
FLEKIEEAQEEHERYHNNWKNLADTYGLPQIVAKEIVAMCPKCQIKGEPVHGQVDASPGTWQMDCTHLEGKVVIVAVHVASGFIEAEVIPRETGKETAKFLLKILSRWPITQLHTDNGPNFTSQEVAAICWWGKIEHTTGIPYNPQSQGSIESMNKQLKEIIGKIRDDCQYTETAVLMACHIHNFKRKGGIGGQTSAERLINIITTQLEIQHLQTKIQKILNFRVYYREGRDPVWKGPAQLIWKGEGAVVLKDGSDLKVVPRRKAKIIKDYEPKQRVGNEGDVEGTRGSDN

SEQ ID NO: 29 - protein, 519 aa, synthetic construct
MGAATSALNRRQLDQFEKIRLRPNGKKKYQIKHLIWAGKEMERFGLHERLLETEEGCKRIIEVLYPLEPTGSEGLKSLFNLVCVLYCLHKEQKVKDTEEAVATVRQHCHLVEKEKSATETSSGQKKNDKGIAAPPGGSQNFPAQQQGNAWVHVPLSPRTLNAWVKAVEEKKFGAEIVPMFQALSEGCTPYDINQMLNVLGDHQGALQIVKEIINEEAAQWDVTHPLPAGPLPAGQLRDPRGSDIAGTTSSVQEQLEWIYTANPRVDVGAIYRRWIILGLQKCVKMYNPVSVLDIRQGPKEPFKDYVDRFYKAIRAEQASGEVKQWMTESLLIQNANPDCKVILKGLGMHPTLEEMLTACQGVGGPSYKAKVMAEMMQTMQNQNMVQQGGPKRQRPPLRCYNCGKFGHMQRQCPEPRKTKCLKCGKLGHLAKDCRGQVNFLGYGRWMGAKPRNFPAATLGAEPSAPPPPSGTTPYDPAKKLLQQYAEKGKQLREQKRNPPAMNPDWTEGYSLNSLFGEDQ

SEQ ID NO: 30 - protein, 1044 aa, synthetic construct
MSKVWKIGTPSKRLQGTGEFFRVWTVDGGKTEKFSRRYSWSGTECASSTERHHPIRPSKEAPAAICRERETTEGAKEESTGNESGLDRGIFFELPLWRRPIKTVYIEGVPIKALLDTGADDTIIKENDLQLSGPWRPKIIGGIGGGLNVKEYNDREVKIEDKILRGTILLGATPINIIGRNLLAPAGARLVMGQLSEKIPVTPVKLKEGARGPCVRQWPLSKEKIEALQEICSQLEQEGKISRVGGENAYNTPIFCIKKKDKSQWRMLVDFRELNKATQDFFEVQLGIPHPAGLRKMRQITVLDVGDAYYSIPLDPNFRKYTAFTIPTVNNQGPGIRYQFNCLPQGWKGSPTIFQNTAASILEEIKRNLPALTIVQYMDDLWVGSQENEHTHDKLVEQLRTKLQAWGLETPEKKVQKEPPYEWMGYKLWPHKWELSRIQLEEKDEWTVNDIQKLVGKLNWAAQLYPGLRTKNICKLIRGKKNLLELVTWTPEAEAEYAENAEILKTEQEGTYYKPGIPIRAAVQKLEGGQWSYQFKQEGQVLKVGKYTKQKNTHTNELRTLAGLVQKICKEALVIWGILPVLELPIEREVWEQWWADYWQVSWIPEWDFVSTPPLLKLWYTLTKEPIPKEDVYYVDGACNRNSKEGKAGYISQYGKQRVETLENTTNQQAELTAIKMALEDSGPNVNIVTDSQYAMGILTAQPTQSDSPLVEQIIALMIQKQQIYLQWVPAHKGIGGNEEIDKLVSKGIRRVLFLEKIEEAQEEHERYHNNWKNLADTYGLPQIVAKEIVAMCPKCQIKGEPVHGQVDASPGTWQMDCTHLEGKVVIVAVHVASGFIEAEVIPRETGKETAKFLLKILSRWPITQLHTDNGPNFTSQEVAAICWWGKIEHTTGIPYNPQSQGSIESMNKQLKEIIGKIRDDCQYTETAVLMACHIHNFKRKGGIGGQTSAERLINIITTQLEIQHLQTKIQKILNFRVYYREGRDPVWKGPAQLIWKGEGAVVLKDGSDLKVVPRRKAKIIKDYEPKQRVGNEGDVEGTRGSDN

(19) United States
(12) Patent Application Publication
(10) Pub. No.: US 2024/0024515 A1
(43) Pub. Date: Jan. 25, 2024

(54) COMBINATION TREATMENT

(71) Applicants: Imperial College of Science, Technology and Medicine, London (GB); The Chancellor, Masters and Scholars of The University of Oxford, Oxford (GB); The University Court of The University of Edinburgh, Edinburgh (GB); IP2IPO Innovations Limited, London (GB); Boehringer Ingelheim International GmbH, Ingelheim am Rhein (DE)

(72) Inventors: Eric Alton, London (GB); Jane Davies, London (GB); Uta Grisenbach, London (GB); Ana Sergijenko, London (GB); Deborah R. Gill, Oxford (GB); Stephen C. Hyde, Oxford (GB); Christopher Boyd, Edinburgh (GB); Gerry McLachlan, Midlothian (GB); Sebastian Kreuz, Ingelheim am Rhein (DE); Alena Moiseenko, Ingelheim am Rhein (DE)

(21) Appl. No.: 18/298,882

(22) Filed: Apr. 11, 2023

(30) Foreign Application Priority Data
Apr. 11, 2022 (GB) GB2205317.7
Aug. 30, 2022 (GB) GB2212566.0

(51) Int. Cl. A61K 48/00; A61K 38/17; A61K 31/4439; A61K 31/47; A61K 31/443; A61K 31/404; A61P 11/00; C12N 15/86

(52) U.S. Cl. CPC A61K 48/0066; A61K 48/0041; A61K 38/177; A61K 31/4439; A61K 31/47; A61K 31/443; A61K 31/404; A61P 11/00; C12N 15/86; C12N 2740/15043; C12N 2740/15071

(57) ABSTRACT

The present invention relates to combination treatments for cystic fibrosis, particularly combinations of modulators of the Cystic Fibrosis Transmembrane Conductance Regulator (CFTR) and gene therapy.

### CROSS-REFERENCE

This application claims priority to GB Patent Application Nos. GB2205317.7, filed Apr. 11, 2022, and GB2212566.0, filed Aug. 30, 2022; which are incorporated herein by reference in their entirety.

### SEQUENCE LISTING

The instant application contains a Sequence Listing which has been submitted electronically in XML format and is hereby incorporated by reference in its entirety. Said XML copy, created on Apr. 11, 2023, is named P71644US_sequence_listing and is 116,792 bytes in size.

### FIELD OF THE INVENTION

The present invention relates to combination treatments for cystic fibrosis, particularly combinations of modulators of the Cystic Fibrosis Transmembrane Conductance Regulator (CFTR) and gene therapy.

### BACKGROUND TO THE INVENTION

Cystic fibrosis (CF) is a severe genetic disease, caused by mutations in the CF Transmembrane Conductance Regulator (CFTR) gene. These mutations result in the production of a faulty CFTR protein, the malfunctioning of which affects the balance of salt and fluids inside and outside of the cell. This imbalance leads to thick, sticky mucus in the lungs, pancreas, and other organs.

Current treatments for CF include CFTR modulator therapy, which attempts to correct the CFTR malfunctions. These modulator drugs have the ability to enhance or even restore the functional expression of specific CF-causing mutations. These CFTR modulator drugs have been classified into five main groups depending on their effects on CFTR mutations: potentiators, correctors, stabilizers, read-through agents, and amplifiers. To date, four CFTR modulators have reached the market, Kalydeco® (ivacaftor), Orkambi® (lumacaftor/ivacaftor), Symdeko® (tezacaftor/ivacaftor) and Trikafta® (elexacaftor/tezacaftor/ivacaftor).

CFTR modulators offer significant improvements for many CF patients, but approximately 10% remain modulator-insensitive or intolerant. In particular, despite the recent successes of CFTR channel modulators an unmet need remains for those patients unable to tolerate side effects of ion channel modulator therapy, or subsets still lacking disease-modifying treatment options such as patients affected by homozygous Class I mutations.

The principal cause of morbidity and mortality in CF is pulmonary disease. Since the cloning of the CFTR gene in 1989, there has been significant interest in the possibility of gene therapy as a treatment for CF. However, gene transfer efficiency to the airway epithelium is generally poor, at least in part because the respective receptors for many viral vectors appear to be predominantly localised to the basolateral surface of the airway epithelium. These vectors can also have difficulty in overcoming the body's host defences, and there remain difficulties with producing efficient expression after readministration. As a result of these difficulties, whilst several gene therapy approaches for CF including adenovirus, adeno-associated virus (AAV) and plasmid-based vectors have been investigated in clinical trials to-date, none have progressed to market authorization so far, largely due to concerns regarding their limited efficiency. In addition, the ability to administer conventional viral vectors repeatedly, mandatory for the life-long treatment of a self-renewing epithelium, is limited, because of patients' adaptive immune responses, which prevent successful repeat administration.

There is accordingly a need for new and effective therapies for CF, particularly for patients who are CF modulator-insensitive or intolerant, or who lack disease-modifying treatment options. In particular, it is an object of the invention to provide new therapies which can combine existing CF modulators, particularly CFTR potentiators, with CF gene therapy, with the potential to maximise the benefits associated with CF gene therapy. Combination therapies may also potentially address some of the disadvantages associated with the current treatments, including modulator insensitivity/tolerance, and/or poor gene transfer efficiency of CF gene therapy vectors.

### SUMMARY OF THE INVENTION

The present inventors have now shown that the combination of a CFTR modulator, particularly a CFTR potentiator, and a lentiviral gene therapy vector together is not only able to drive CTFR expression, but also improve CTFR function and restore airway cell function. In particular, using air-liquid interface (ALI) cultures of two different CFTR mutational backgrounds (class I and class II) cells, the inventors have demonstrated that the combination of (i) ivacaftor or (ii) ivacaftor-containing combinations, and a SIV vector pseudotyped with hemagglutinin-neuraminidase (HN) and fusion (F) proteins from a respiratory paramyxovirus (rSIV.F/HN) containing the CFTR transgene is able to drive CFTR expression in ALI models of both class I and class II CFTR mutations, restoring CFTR chloride current and increasing ciliary beat frequency. The present inventors are the first to show that the combination of a CFTR modulator and a CF gene therapy, particularly using a rSIV.F/HN vector can achieve functional correction in a class I CFTR mutation model, where the class I null mutation results in complete absence of full-length CFTR protein and is thus typically not amenable to functional correction using CFTR modulators alone. Thus, the inventors have demonstrated a beneficial and unexpected effect between CFTR modulators, particularly potentiators, and rSIV.F/HN for at least class I and class II CFTR mutations. Even more surprisingly, the present inventors have demonstrated that CFTR modulators, particularly CFTR potentiators such as those including ivacaftor, achieve a greater than expected potentiation of the CFTR transgene expressed by rSIV.F/HN. In particular, the effect of a CFTR modulator, particularly a CFTR potentiator, and rSIV.F/HN-CFTR combination is greater than the additive effects of the separate effects of the CFTR modulator/potentiator and rSIV.F/HN-mediated CFTR expression. This is exemplified herein with the CFTR potentiator ivacaftor and rSIV.F/HN-CFTR, achieving greater than the additive effects of the separate effects of ivacaftor and rSIV.F/HN-mediated CFTR expression. Therefore, the present inventors are the first to have demonstrated the advantageous therapeutic potential of the combination of CFTR modulators and rSIV.F/HN-based CF gene therapy, particularly for patients with class I CFTR mutations, or for patients who are otherwise CFTR modulator-insensitive, intolerant, or poorly responding.

Accordingly, the present invention provides a combination of (i) a lentiviral vector pseudotyped with hemagglutinin-neuraminidase (HN) and fusion (F) proteins from a respiratory paramyxovirus, wherein said lentiviral vector comprises a cystic fibrosis transmembrane conductance regulator (CFTR) transgene and (ii) a CFTR modulator, for use in a method of treating cystic fibrosis (CF).

The lentiviral vector may be a SIV vector and the respiratory paramyxovirus may be a Sendai virus. The transgene may be operably linked to a promoter selected from the group consisting of a cytomegalovirus (CMV) promoter, elongation factor 1a (EF1a) promoter, and a hybrid human CMV enhancer/EF1a (hCEF) promoter. The lentiviral vector may comprise a hybrid human CMV enhancer/EF1a (hCEF) promoter, which optionally comprises or consist of a nucleotide sequence having at least 90% identity to SEQ ID NO: 2. The CFTR transgene may be a codon-optimised CFTR transgene, which optionally comprises or consists of a nucleotide sequence having at least 90% identity to SEQ ID NO: 1. The lentiviral vector may be produced using codon-optimised plasmids. The lentiviral vector may be produced using (i) pGM691 (SEQ ID NO: 7) and/or (ii) pGM830 (SEQ ID NO: 9) or pGM326 (SEQ ID NO: 8); and preferably also using pGM299 (SEQ ID NO: 11), pGM301 (SEQ ID NO: 12) and/or pGM303 (SEQ ID NO: 13). The lentiviral vector may be vGM058, vGM195 or vGM244. The lentiviral vector may be an SIV vector pseudotyped with Sendai virus hemagglutinin-neuraminidase (HN) and fusion (F) proteins, wherein: said vector comprises a modified retroviral RNA sequence which comprises or consists of a nucleic acid sequence of SEQ ID NO: 16. The lentiviral vector may comprise an F protein with a first subunit which comprises or consists of an amino acid sequence of SEQ ID NO: 19 and a second subunit which comprises or consists of an amino acid sequence of SEQ ID NO: 20. The lentiviral vector may further comprise: (a) a p17 protein comprising or consisting of an amino acid sequence of SEQ ID NO: 22; (b) a p24 protein comprising or consisting of an amino acid sequence of SEQ ID NO: 23; (c) a p8 protein comprising or consisting of an amino acid sequence of SEQ ID NO: 24; (d) a protease comprising or consisting of an amino acid sequence of SEQ ID NO: 25; (e) a p51 protein comprising or consisting of an amino acid sequence of SEQ ID NO: 26; (f) a p15 protein comprising or consisting of an amino acid sequence of SEQ ID NO: 27; (g) a p31 protein comprising or consisting of an amino acid sequence of SEQ ID NO: 28; (h) a Gag protein comprising or consisting of an amino acid sequence of SEQ ID NO: 29; and/or (i) a Pol protein comprising or consisting of an amino acid sequence of SEQ ID NO: 30; wherein optionally the vector comprises each of (a) to (g).

The CFTR modulator may be a CFTR potentiator and/or a CFTR corrector, preferably a CFTR potentiator. The CFTR modulator may be selected from ivacaftor, tezacaftor, elexacaftor or lumacaftor, or a combination thereof. Preferably the CFTR modulator is ivacaftor.

The invention provides the combination of (A) an SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 16; and (b) the F protein comprises a first subunit which comprises or consists of an amino acid sequence of SEQ ID NO: 19 and a second subunit which comprises or consists of an amino acid sequence of SEQ ID NO: 20; and (B) ivacaftor; for use in a method of treating cystic fibrosis (CF). In said combination, the vector may further comprise one or more of: (a) a p17 protein comprising or consisting of an amino acid sequence of SEQ ID NO: 22; (b) a p24 protein comprising or consisting of an amino acid sequence of SEQ ID NO: 23; (c) a p8 protein comprising or consisting of an amino acid sequence of SEQ ID NO: 24; (d) a protease comprising or consisting of an amino acid sequence of SEQ ID NO: 25; (e) a p51 protein comprising or consisting of an amino acid sequence of SEQ ID NO: 26; (f) a p15 protein comprising or consisting of an amino acid sequence of SEQ ID NO: 27; (g) a p31 protein comprising or consisting of an amino acid sequence of SEQ ID NO: 28; (h) a Gag protein comprising or consisting of an amino acid sequence of SEQ ID NO: 29; and/or (i) a Pol protein comprising or consisting of an amino acid sequence of SEQ ID NO: 30; wherein optionally the vector comprises each of (a) to (g).

A patient to be treated may have at least one class I, class II, class III, class IV, class V and/or class VI CFTR mutation. The patient be treated may have at least one class I and/or class II CFTR mutation. The combination treatment of the invention may be suitable for use independent of the CFTR mutation of the patient. The patient to be treated may have: (a) at least one class I CFTR mutation selected from G542X, W1282X and/or R553C; and/or (b) at least one class II CFTR mutation selected from F508del, N1303K and/or I507del.

The lentiviral vector and the CFTR modulator may be administered simultaneously or sequentially. The lentiviral vector may be administered by inhalation; and/or the CFTR modulator may be administered orally. The lentiviral vector may be administered at a dose of between about 8⁸ to about 10¹⁴ transducing units (TU), preferably a dose of between about 10⁶ to about 10¹² TU, wherein optionally the lentiviral vector is administered at a frequency of every 3 months, every 6 months, every 12 months, every 24 months, every 36 months or every 48 months; and/or the CFTR modulator may be administered at a concentration used for monotherapy of each modulator or lower.

Treatment may restore CFTR activity to at least 10% of CFTR activity in a healthy control. Treatment may restore CFTR activity to at least 50% of CFTR activity in a healthy control. Treatment may increase CFTR activity by at least 1.2 fold compared with treatment with the lentiviral vector alone. Treatment may increase CFTR current by about 1.3 fold to about 3 fold or about 1.3 fold to about 1.8 fold compared with treatment with the lentiviral vector alone. The patient to be treated may have a class I CFTR mutation and the treatment may: (i) restore CFTR activity to at least 10% of CFTR activity in a healthy control; and/or (ii) increase CFTR current by about 1.3 fold to about 1.8 fold or about 1.3 fold to about 3 fold compared with treatment with the lentiviral vector alone. The patient to be treated may have a class II CFTR mutation and the treatment may: (i) restore CFTR activity to at least 10% of CFTR activity in a healthy control; and/or (ii) increase CFTR current by about 1.3 fold to about 3 fold or about 1.3 fold to about 1.8 fold compared with treatment with the lentiviral vector alone. A transduction rate of between about 10% to about 20%, preferably between about 14% to about 17% may be sufficient to achieve a therapeutic effect on CFTR activity as defined herein.

The invention also provides a method of treating CF in a subject in need thereof, comprising administering to the subject a therapeutically effective amount of each of (i) a lentiviral vector pseudotyped with hemagglutinin-neuraminidase (HN) and fusion (F) proteins from a respiratory paramyxovirus, wherein said lentiviral vector comprises a cystic fibrosis transmembrane conductance regulator (CFTR) transgene and (ii) a CFTR modulator.

The invention further provides the use of a lentiviral vector pseudotyped with hemagglutinin-neuraminidase (HN) and fusion (F) proteins from a respiratory paramyxovirus, wherein said lentiviral vector comprises a cystic fibrosis transmembrane conductance regulator (CFTR) transgene in the manufacture of a medicament for use in a method of treating CF, wherein said method further comprises administration of a CFTR modulator.

### BRIEF DESCRIPTION OF THE DRAWINGS

FIGS. 1A-G show schematic drawings of exemplary plasmids used for production of the vectors of the invention.

FIGS. 2A-F. Transduction efficiency and transduced cell types in HBEC ALI (FIG. 2A) Experimental setup. (FIG. 2B) Quantification of transduced GFP+ cells at day 21 post-transduction. (FIG. 2C) Immunofluorescence for ciliated (ACTUB), basal (KRT5), club (SCGB1A1) and goblet (MUC5AC) cells and co-localization with transduced GFP+ cells. Arrow indicates co-localization. (FIG. 2D) Flow cytometry quantification of percentage of transduced cells in specific epithelial cell population. (FIG. 2E) Vector copy number (VCN) qPCR analysis in bulk samples transduced either with GFP- or with CFTR-expressing rSIV.F/HN. (FIG. 2F) Transgene mRNA (RT-ddPCR) on sorted single cells. MOI—multiplicity of infection, WPRE—woodchuck hepatitis post-transcriptional regulatory element. Scale bar in C −20 μm. Differences in VCN were analyzed with two-way ANOVA (***p<0.001, ****p<0.0001).

FIGS. 3A-H. Functional data demonstrating that rSIV.F/HN (vGM058) restores CFTR chloride current in primary CF HBECs (class II). (FIG. 3A) Codon optimized CFTR transgene gene expression showing high expression of transgene in CFTR-transduced cells but not in the negative control GFP-transduced cells. (FIG. 3B) Endogenous human CFTR gene expression. (FIG. 3C) Gene expression ratio between transgene coCFTR and endogenous hCFTR. (FIG. 3D) Representative scheme of Ussing chamber measurements. (FIG. 3E) Ussing chamber data represented as the percentage of WT CFTR current; difference between forskolin peak and CFTR-inhibited trough current is calculated. (FIG. 3F) Ussing chamber data represented as percentage of WT CFTR current; difference between Forskolin plateau and CFTR-inhibited trough current is calculated (FIG. 3G) Correlation between the percentage of cells expressing CFTR and CFTR chloride current restoration in primary CF HBECs. (FIG. 3H) Mean ciliary beat frequency analysis in primary non-CF, CF and transduced CF HBECs at day 28 post airlift. Δlsc—short circuit current change, UC—Ussing chamber, Amil—amiloride, Fsk—forskolin, Iva—ivacaftor, Luma—lumacaftor, Teza—tezacaftor, Elexa—elexacaftor, Hz—Hertz. Differences in Δlsc current and mean cilia beat frequency were analyzed with one-way ANOVA (*p<0.05, **p<0.005, ***p<0.001, ****p<0.0001) For Ussing chamber experiments N=16-26 for most of the conditions except for MOI 30 and 90 (N=4). Stars on top of the graph indicate statistical significance in comparison to CF MOI 0 control.

FIGS. 4A-E. Generation of CFTR KO (CFTR null, class I) hSABCi cell line and assessment of transduction with rSIV.F/HN-GFP (vGM107) and rSIV.F/HN-CFTR (vGM058). (FIG. 4A) CFTR protein expression in hSBACi (originally described in Wang et al. Respir. Res. (2019) 20:196) and CFTR KO cells from Clone 5 with the highest editing efficiency. (FIG. 4B) Flow cytometry quantification of transduced GFP+ CFTR KO cells at day 21 post-transduction. (FIG. 4C) Vector copy number (VCN) qPCR analysis in bulk CFTR KO samples transduced with GFP- and CFTR-expressing vectors. (FIG. 4D) Immunofluorescence for ciliated (ACTUB), basal (KRT5), club (SCGB1A1) and goblet (MUC5AC) cells and co-localisation with transduced GFP+ cells. Arrow indicates co-localization. (FIG. 4E) Flow cytometry quantification of percentage of transduced cells in specific epithelial cell populations. MOI—multiplicity of infection, FITC-A—Fluorescein isothiocyanate area, SSC-A—side scatter area. Scale bar in E −20 μm. Differences in VCN were analyzed with two-way ANOVA (**p<0.01, ****p<0.0001).

FIGS. 5A-D. Functional data demonstrating that rSIV.F/HN (vGM058) restores CFTR chloride current in CFTR KO cells (Class I) in contrast to modulators. (FIG. 5A) Codon optimised CFTR transgene gene expression showing high expression of transgene in CFTR-transduced cells, but not in GFP-transduced cells. (FIG. 5B) Ussing chamber data represented as percentage of WT CFTR current; difference between Forskolin peak and CFTR-inhibited trough current is calculated. Of note, there was no CFTR chloride current activation after treatment with modulators (Luma+Iva, Teza+Iva, Elexa+Teza+Iva) (FIG. 5C) Ussing chamber data represented as percentage of WT CFTR current; difference between Forskolin plateau and CFTR-inhibited trough current is calculated. Of note there was no CFTR chloride current activation after treatment with modulators (Luma+Iva, Teza+Iva, Elexa+Teza+Iva). (FIG. 5D) Correlation between the percentage of cells expressing CFTR and restoration of the CFTR chloride current in transduced CFTR KO cells. Δlsc—short circuit current change, Iva—ivacaftor, Luma—lumacaftor, Teza—tezacaftor, Elexa—elexacaftor. Differences in Δlsc were analyzed with one-way ANOVA (*p<0.05, **p<0.01, ***p<0.001, ****p<0.0001). For Ussing chamber experiments N=5-11. Stars on top of the graph indicate statistical significance in comparison to CF MOI 0 control.

FIGS. 6A-D. Comparison of vGM058 and vGM244: VCN, coCFTR expression and functional correction levels. (FIG. 6A) Vector copy number (VCN) ddPCR analysis in bulk samples transduced either with vGM107, vGM058 or vGM244 rSIV.F/HN. (FIG. 6B) Codon optimised CFTR transgene gene expression in samples transduced either with vGM107, vGM058 or vGM244 rSIV.F/HN. (FIG. 6C) Ussing chamber data represented as the percentage of WT CFTR current; difference between maximum forskolin peak current and CFTR-inhibited trough current is calculated. (FIG. 6D) Ussing chamber data represented as percentage of WT CFTR current; difference between Forskolin plateau and CFTR-inhibited trough current is calculated. WPRE—woodchuck hepatitis post-transcriptional regulatory element. ddPCR data were analyzed using Mann-Whitney test, Ussing chamber differences were analyzed with two-way ANOVA (*p<0.05).

FIGS. 7A-B. DNA vector copy number and coCFTR RNA expression in class II cells transduced with vGM0244 and vGM107. (FIG. 7A) Vector copy number (VCN) qPCR analysis in bulk samples transduced either with GFP- or with CFTR-expressing rSIV.F/HN. (FIG. 7B) Codon optimised CFTR transgene gene expression showing high expression of transgene in CFTR-transduced cells, but not in GFP-transduced cells. WPRE—woodchuck hepatitis post-transcriptional regulatory element. Differences were analysed with two-way ANOVA (****p<0.0001).

FIGS. 8A-B. Functional data demonstrating that rSIV.F/HN (vGM244) restores CFTR chloride current in primary CF HBECs (class II). (FIG. 8A) Ussing chamber data represented as the percentage of WT CFTR current; difference between maximum forskolin peak current and CFTR-inhibited trough current calculated. (FIG. 8B) Ussing chamber data represented as percentage of WT CFTR current; difference between Forskolin plateau and CFTR-inhibited trough current is calculated. Teza—tezacaftor, Elexa—elexacaftor. Differences were analysed with one-way ANOVA (**p<0.01, ***p<0.001, ****p<0.0001). For Ussing chamber experiments N=25-36, for most of the conditions except for MOI 90 (N=12).

FIG. 9. Transduction with vGM244 results in restoration of ciliary beat frequency in Class II CF HBECs. Mean ciliary beat frequency analysis in primary non-CF, CF and transduced CF HBECs at day 28 post airlift. Hz—Hertz. Differences were analyzed with one-way ANOVA (*p<0.05, **p<0.005, ***p<0.001, ****p<0.0001).

FIGS. 10A-B. DNA vector copy number and coCFTR RNA expression in class I cells transduced with vGM244 and vGM107. (FIG. 10A) Vector copy number (VCN) qPCR analysis in bulk samples transduced either with GFP- or with CFTR-expressing rSIV.F/HN. (FIG. 10B) Codon optimised CFTR transgene gene expression showing high expression of transgene in CFTR-transduced cells, but not in GFP-transduced cells. WPRE—woodchuck hepatitis post-transcriptional regulatory element. Differences were analysed with two-way ANOVA ((*p<0.05, **p<0.005, ****p<0.0001).

FIGS. 11A-B. Functional data demonstrating that rSIV.F/HN (vGM244) restores CFTR chloride current in primary CF HBECs (class II). (FIG. 11A) Ussing chamber data represented as the percentage of WT CFTR current; difference between maximum forskolin peak current and CFTR-inhibited trough current is calculated. (FIG. 11B) Ussing chamber data represented as percentage of WT CFTR current; difference between Forskolin plateau and CFTR-inhibited trough current is calculated. Teza—tezacaftor, Elexa—elexacaftor. Differences were analysed with one-way ANOVA (*p<0.05, **p<0.005, ***p<0.001, ****p<0.0001). For Ussing chamber experiments N=10-18, for most of the conditions except for MOI 90 (N=4).

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Amino acids are referred to herein using the name of the amino acid, the three letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide typically comprises at least 50%, at least 60%, at least 70%, at least 80%, at least 90%, at least 95%, at least 97% or more of the original polypeptide.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a increase, or any increase between 2-fold and 10-fold or greater as compared to a reference level. In the context of a yield or titre, an “increase” is an observable or statistically significant increase in such level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

It must be noted that as used herein and in the appended claims, the singular forms “a”, “an”, and “the” include plural referents unless the context clearly dictates otherwise. Thus, for example, reference to “a CFTR modulator” includes a plurality of such agents and reference to “the CFTR modulator” includes reference to one or more CFTR modulators and equivalents thereof known to those skilled in the art, and so forth. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the terms “vector”, “retroviral vector” and “retroviral F/HN vector” are used interchangeably to mean a retroviral vector comprising a retroviral RNA sequence and pseudotyped with hemagglutinin-neuraminidase (HN) and fusion (F) proteins from a respiratory paramyxovirus, unless otherwise stated. The terms “lentiviral vector” and “lentiviral F/HN vector” are used interchangeably to mean a lentiviral vector pseudotyped with hemagglutinin-neuraminidase (HN) and fusion (F) proteins from a respiratory paramyxovirus, unless otherwise stated. All disclosure herein in relation to retroviral vectors of the invention applies equally and without reservation to lentiviral vectors of the invention and to SIV vectors that are pseudotyped with hemagglutinin-neuraminidase (HN) and fusion (F) proteins from a respiratory paramyxovirus (also referred to herein as SIV F/HN or SIV-FHN).

As defined herein, the term “retroviral RNA sequence” refers to the nucleic acid molecule that is contained within a retroviral vector. A retroviral RNA sequence comprises long terminal repeat (LTR) elements, nucleic acid sequences necessary for incorporation of the retroviral RNA sequence into retroviral particles, and the transgene expression cassette. The transgene expression cassette is comprised of a suitable enhancer/promoter element, the transgene cDNA and a posttranscriptional regulatory element. The retroviral RNA sequence starts with a 5′ LTR R sequence and ends with a 3′ LTR R sequence. The 5′ region retroviral RNA sequence typically comprises or consists of a retroviral LTR R sequence followed by a retroviral LTR U5 sequence (in 5′ to 3′ order). The 3′ region retroviral RNA sequence typically comprises or consists of a retroviral LTR R sequence followed by a retroviral LTR U5 sequence (in 5′ to 3′ order).

The terms “DNA provirus” or “DNA provirus sequence” and “DNA proviral sequence” refer interchangeably to the DNA sequence which is integrated into the genome of cells transduced with the retrovirus. The DNA provirus sequence contains additional regions of nucleic acid that are not found within the retroviral RNA sequence, including a 5′ LTR U3 sequence and a 3′ LTR U5 sequence. Therefore, the sequences of the DNA provirus and the retroviral RNA sequence are not identical, but rather the sequence of the retroviral RNA sequence is shorter than the proviral DNA sequence from which it is derived. The precise 5′ and 3′ limits of the retroviral RNA sequence compared with the proviral DNA sequence from which it is derived cannot readily and reliably be determined by simple analysis of the proviral DNA sequence.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

The publications discussed herein are provided solely for their disclosure prior to the filing date of the present application. Nothing herein is to be construed as an admission that such publications constitute prior art to the claims appended hereto. All references cited in this specification are herewith incorporated by reference with respect to their entire disclosure content and the disclosure content specifically mentioned in this specification.

Disclosure related to the various methods of the invention are intended to be applied equally to other methods, therapeutic uses or methods, and vice versa.

### Retroviral and Lentiviral Vectors

The invention relates to combination therapies comprising a retroviral/lentiviral (e.g. SIV) construct. The term “retrovirus” refers to any member of the Retroviridae family of RNA viruses that encode the enzyme reverse transcriptase. The term “lentivirus” refers to a family of retroviruses. Examples of retroviruses suitable for use in the present invention include gammaretroviruses such as murine leukaemia virus (MLV) and feline leukaemia virus (FLV). Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. Typically the invention relates to combination therapies comprising lentiviral vectors, particularly combination therapies comprising an SIV vector (including all strains and subtypes), such as a SIV-AGM (originally isolated from African green monkeys, Cercopithecus aethiops).

The retroviral/lentiviral (e.g. SIV) vectors of the present invention are pseudotyped with hemagglutinin-neuraminidase (HN) and fusion (F) proteins from a respiratory paramyxovirus. Preferably the respiratory paramyxovirus is a Sendai virus (murine parainfluenza virus type 1).

The F protein may be a truncated F protein, typically one in which the cytoplasmic domain is truncated. Preferably the truncated F protein is Fct4, in which 38 amino acids have been truncated from the C-terminus of the F protein, with 4 amino acids of the F protein cytoplasmic domain being retained. Thus, the F protein may comprise or consist of an Fct4 amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 17 or 18. Preferably the F protein may comprise or consist of an Fct4 amino acid sequence having at least 90%, at least 95%, or at least 99% identity to SEQ ID NO: 17 or 18.

The full length F protein, or C-terminally truncated form thereof (e.g. Fct4) is typically fusion inactive. The fusion inactive form of the F protein may be cleaved to produce two subunits, a first subunit, (also known as F₂) and a second subunit (also known as F₁).

The first subunit of the F protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 19. Preferably the first subunit may be a subunit which may comprises or consists of an amino acid sequence having at least 90%, at least 95%, or at least 99% identity to SEQ ID NO: 19. SEQ ID NO: 19 is the first subunit of Fct4.

Alternatively or in addition, preferably in addition, the second subunit of the F protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 20. Preferably the second subunit may be a subunit which may comprises or consists of an amino acid sequence having at least 90%, at least 95%, or at least 99% identity to SEQ ID NO: 20. SEQ ID NO: 20 is the second subunit of Fct4.

The F protein (e.g. Fct4) may comprise an N-terminal signal peptide. Alternatively, the F protein may lack such a signal peptide. The F protein signal peptide may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to SEQ ID NO: 21. This signal peptide may be cleaved to form the mature F protein. The signal peptide of Fct4 is SEQ ID NO: 21, which forms amino acid residues 1-25 of SEQ ID NO: 18. Thus, the mature form of Fct4 may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% identity to amino acid residues 26-527 of SEQ ID NO: 18.

The HN protein may be a truncated and/or chimeric HN protein, typically one in which the cytoplasmic domain is truncated or substituted. Preferably, the HN protein is a chimeric HN protein in which (i) the cytoplasmic domain of the HN is replaced by the cytoplasmic domain of the transmembrane (TMP) protein; or (ii) the cytoplasmic domain of the TMP is added to the cytoplasmic domain of the HN protein. The HN protein may be as described in Kobayashi et al. (J. Virol. (2003) 77(4):2607-2614), which is herein incorporated by reference in its entirety.

The retroviral/lentiviral (e.g. SIV) vectors of the invention may comprise a codon-optimised Gag protein, a codon-optimised Pol protein, a codon-optimised GagPol polyprotein, or a combination thereof. Accordingly, the invention provides a retroviral/lentiviral (e.g. SIV) vector comprising a codon-optimised Gag protein comprising or consisting of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 29. Preferably, the invention provides a retroviral vector comprising a codon-optimised Gag protein comprising or consisting of an amino acid sequence having at least 90%, at least 95%, or at least 99% identity to SEQ ID NO: 29. The invention provides a retroviral vector comprising a codon-optimised Pol protein comprising or consisting of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 30. Preferably, the invention provides a retroviral vector comprising a codon-optimised Pol protein comprising or consisting of an amino acid sequence having a at least 90%, at least 95%, or at least 99% sequence identity to SEQ ID NO: 30.

GagPol is expressed as polyprotein which is processed to produce a number of smaller proteins within viral particles. The extent of processing, and hence the presence and/or concentration of GagPol or any of the constituent proteins within a retroviral/lentiviral (e.g. SIV) vector of the invention may vary with time.

Accordingly, a retroviral/lentiviral (e.g. SIV) vector of the invention may comprise one or more of a p17 protein, a p27 protein, a p8 protein, a protease, a p51 protein, a p15 protein and a p31 protein. One or more of these proteins may be present in combination with Gag, Pol and/or GagPol. Preferably, the invention provides a retroviral vector comprising a p17 protein, a p27 protein, a p8 protein, a protease, a p51 protein, a p15 protein and a p31 protein. Again, these proteins may be present in combination with Gag, Pol and/or GagPol.

The p17 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 22. Preferably, the p17 protein comprises or consists of an amino acid sequence having at least 90%, at least 95%, or at least 99% sequence identity to SEQ ID NO: 22.

The p24 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 23. Preferably, the p24 protein comprises or consists of an amino acid sequence having at least 90%, at least 95%, or at least 99% sequence identity to SEQ ID NO: 23.

The p8 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 24. Preferably, the p8 protein comprises or consists of an amino acid sequence having at least 90%, at least 95%, or at least 99% sequence identity to SEQ ID NO: 24.

The protease may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 25. Preferably, the protease comprises or consists of an amino acid sequence having at least 90%, at least 95%, or at least 99% sequence identity to SEQ ID NO: 25.

The p51 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 26. Preferably, the p51 protein comprises or consists of an amino acid sequence having at least 90%, at least 95%, or at least 99% sequence identity to SEQ ID NO: 26.

The p15 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 27. Preferably, the p15 protein comprises or consists of an amino acid sequence having at least 90%, at least 95%, or at least 99% sequence identity to SEQ ID NO: 27.

The p31 protein may comprise or consist of an amino acid sequence having at least 70%, at least 80%, at least 90%, at least 91%, at least 92%, at least 93%, at least 94%, at least 95%, at least 96%, at least 97%, at least 98%, at least 99%, at least 99.5%, at least 99.9%, or more, up to 100% sequence identity to SEQ ID NO: 28. Preferably, the p31 protein comprises or consists of an amino acid sequence having at least 90%, at least 95%, or at least 99% sequence identity to SEQ ID NO: 28.

Retroviral/lentiviral (e.g. SIV) vectors of the invention may comprise a p17 protein comprising or consisting of an amino acid sequence having at least 90% sequence identity to SEQ ID NO: 22 (as described above), a p24 protein comprising or consisting of an amino acid sequence having at least 90% sequence identity to SEQ ID NO: 23 (as described above), a p8 protein comprising or consisting of an amino acid sequence having at least 90% sequence identity to SEQ ID NO: 24 (as described above), a protease comprising or consisting of an amino acid sequence having at least 90% sequence identity to SEQ ID NO: 25 (as described above), a p51 protein comprising or consisting of an amino acid sequence having at least 90% sequence identity to SEQ ID NO: 26 (as described above), a p15 protein comprising or consisting of an amino acid sequence having at least 90% sequence identity to SEQ ID NO: 27 (as described above), and a p31 protein comprising or consisting of an amino acid sequence having at least 90% sequence identity to SEQ ID NO: 28 (as described above).

A retroviral/lentiviral (e.g. SIV) vector produced according to the invention may be integrase-competent (IC). Alternatively, the lentiviral (e.g. SIV) vector may be integrase-deficient (ID).

Retroviral/Lentiviral vectors, such as those used in combination therapies according to the invention, can integrate into the genome of transduced cells and lead to long-lasting expression, making them suitable for transduction of stem/progenitor cells. In the lung, several cell types with regenerative capacity have been identified as responsible for maintaining specific cell lineages in the conducting airways and alveoli. These include basal cells and submucosal gland duct cells in the upper airways; ciliated, goblet, club cells (SCGB1A1+) and neuroendocrine cells in the bronchiolar airways; bronchioalveolar stem cells in the terminal bronchioles; and type II pneumocytes in the alveoli. Therefore, and without being bound by theory, it is believed that said retroviral/lentiviral (e.g. SIV) vectors bring about long term gene expression of the transgene of interest by introducing the transgene into one or more cell type of the airway epithelium, such as those listed above. It is predicted that cells of the airway epithelium have a lifespan of many months, such that transfection of these cells facilitates expression for the lifespan of the cells, having a long-term therapeutic effect.

Accordingly, the retroviral/lentiviral (e.g. SIV) vectors used in combination therapies according to the invention typically transduce one or more cell types or cell lineages within the airway epithelium. These cells may (or may not) have regenerative potential, but rather prolonged expression results from the long lifespan of the transduced cells. For example, the retroviral/lentiviral (e.g. SIV) vectors may transduce one or more cell type selected from: (i) basal cells and/or submucosal gland duct cells in the upper airways; (ii) ciliated, goblet, club cells (SCGB1A1+) and/or neuroendocrine cells in the bronchiolar airways; (iii) bronchioalveolar stem cells in the terminal bronchioles; and/or (iv) type II pneumocytes in the alveoli; or any combination thereof.

Alternatively or in addition, the retroviral/lentiviral (e.g. SIV) vectors used in combination therapies according to the invention may transduce one or more cell types or cell lineages with regenerative potential within the lung (including the airways and respiratory tract) to achieve long term gene expression. For example, the retroviral/lentiviral (e.g. SIV) vectors may transduce basal cells, such as those in the upper airways/respiratory tract. Basal cells have a central role in processes of epithelial maintenance and repair following injury. In addition, basal cells are widely distributed along the human respiratory epithelium, with a relative distribution ranging from 30% (larger airways) to 6% (smaller airways).

The retroviral/lentiviral (e.g. SIV) vectors may be used to transduce isolated and expanded stem/progenitor cells ex vivo prior administration to a patient as part of a combination therapy as described herein. Preferably, the retroviral/lentiviral (e.g. SIV) vectors are used to transduce cells within the lung (or airways/respiratory tract) in vivo.

The retroviral/lentiviral (e.g. SIV) vectors of the invention demonstrate remarkable resistance to shear forces with only modest reduction in transduction ability when passaged through clinically-relevant delivery devices such as spray bottles and nebulisers. Other inhalative routes of administration, such as by bronchoscope, may similarly benefit from the retroviral/lentiviral (e.g. SIV) vectors of the invention shear force resistance.

A retroviral/lentiviral (e.g. SIV) vector of the invention may comprise one or more transgene that encodes a polypeptide or protein that is therapeutic for the treatment of CF. Preferably a retroviral/lentiviral (e.g. SIV) vector of the invention comprises a CFTR transgene, i.e. the transgene encodes a CFTR.

The transgene included in the vector of the invention may be modified to facilitate expression. For example, the transgene sequence may be in CpG-depleted (or CpG-fee) and/or codon-optimised form to facilitate gene expression. Standard techniques for modifying the transgene sequence in this way are known in the art.

Accordingly, an example of a CFTR cDNA is provided by SEQ ID NO: 1. Variants thereof (as described therein) are also included, particularly variants with at least 90% (such as at least 90, 92, 94, 95, 96, 97, 98, 99 or 100%) sequence identity to SEQ ID NO: 1. SEQ ID NO: 1 is a codon-optimized CpG depleted CFTR transgene previously designed by the present inventors to enhance translation in human cells. Variants of same sequence (as defined herein) which possess the same technical effect of enhancing translation compared with the unmodified (wild-type) CFTR gene sequence are also encompassed by the present invention.

The retroviral/lentiviral (e.g. SIV) vectors of the present invention enable high levels of transgene expression, resulting in high levels (therapeutic levels) of expression of a therapeutic protein. As such, the retroviral/lentiviral (e.g. SIV) vectors of the present invention may usefully provide high expression levels of a transgene when administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or nM.

Expression of a transgene of interest may be given relative to the expression of the corresponding endogenous (defective) gene in a patient. Expression may be measured in terms of DNA vector copy number (VCN), mRNA or protein expression. The expression of the transgene of the invention, such as a functional CFTR gene, may be quantified relative to the endogenous gene, such as the endogenous (dysfunctional) CFTR genes in terms of mRNA copies per cell or any other appropriate unit.

Expression levels of a CFTR transgene and/or the encoded CFTR protein of the invention may be measured in the lung tissue. A high and/or therapeutic expression level may therefore refer to the concentration in the lung, epithelial lining fluid and/or serum/plasma.

The retroviral/lentiviral (e.g. SIV) vectors of the invention exhibit efficient airway cell uptake, stable transgene expression, and suffer no loss of efficacy upon repeated administration. Accordingly, the retroviral/lentiviral (e.g. SIV) vectors of the invention are capable of producing long-lasting, repeatable, high-level expression in airway cells without inducing an undue immune response. An undue immune response may be defined as one extreme enough to preclude administration to a patient and/or to elicit a significant negative effect on vector transduction and/or CFTR expression.

The retroviral/lentiviral (e.g. SIV) vectors of the present invention enable long-term transgene expression, resulting in long-term expression of a therapeutic protein. As described herein, the phrases “long-term expression”, “sustained expression”, “long-lasting expression” and “persistent expression” are used interchangeably. Long-term expression according to the present invention means expression of a therapeutic gene and/or protein, preferably at therapeutic levels, for at least days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more. This long-term expression may be achieved by repeated doses or by a single dose.

Repeated doses may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

The retroviral/lentiviral (e.g. SIV) vector comprises a promoter operably linked to a transgene, enabling expression of the transgene. Typically the promoter is a hybrid human CMV enhancer/EF1a (hCEF) promoter. This hCEF promoter may lack the intron corresponding to nucleotides 570-709 and the exon corresponding to nucleotides 728-733 of the hCEF promoter. A preferred example of an hCEF promoter sequence of the invention is provided by SEQ ID NO: 2. Thus, a hCEF promoter comprised in a retroviral/lentiviral (e.g. SIV) vector of the invention may comprise (or consist of) a nucleic acid sequence having at least 90% (such as at least 90, 92, 94, 95, 96, 97, 98, 99 or 100%) sequence identity to the hCEF nucleic acid sequence of SEQ ID NO: 2. In a further embodiment, the hCEF may comprise (or consist of) a nucleic acid sequence having at least 95% (such as at least 95, 96, 97, 98, 99 or 100%) sequence identity to the hCEF nucleic acid sequence of SEQ ID NO: 2. Alternatively, the promoter may be a CMV promoter. An example of a CMV promoter sequence is provided by SEQ ID NO: 3. The promoter may be a human elongation factor 1a (EF1a) promoter. An example of a EF1a promoter is provided by SEQ ID NO: 4. Other promoters for transgene expression are known in the art and their suitability for the retroviral/lentiviral (e.g. SIV) vectors of the invention determined using routine techniques known in the art. Non-limiting examples of other promoters include UbC and UCOE. As described herein, the promoter may be modified to further regulate expression of the transgene of the invention.

The promoter included in the retroviral/lentiviral (e.g. SIV) vector of the invention may be specifically selected and/or modified to further refine regulation of expression of the therapeutic gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90(12): 1487-96), which is herein incorporated by reference in its entirety. Preferably, the retroviral/lentiviral vectors (particularly SIV F/HN vectors) of the invention comprise a hCEF promoter having low or no CpG dinucleotide content. The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO: 2. The absence of CpG dinucleotides further improves the performance of retroviral/lentiviral (e.g. SIV) vectors of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The retroviral/lentiviral (e.g. SIV) vector of the invention may be modified to allow shut down of gene expression. Standard techniques for modifying the vector in this way are known in the art. As a non-limiting example, Tet-responsive promoters are widely used.

Thus, the invention relates to F/HN retroviral/lentiviral vectors comprising a promoter and a transgene, particularly SIV.F/HN vectors. The F/HN pseudotyping is particularly efficient at targeting cells in the airway epithelium, and as such, for therapeutic applications it is typically delivered to cells of the respiratory tract, including the cells of the airway epithelium. Accordingly, the retroviral/lentiviral (e.g. SIV) vectors of the invention are particularly suited for treatment of CF.

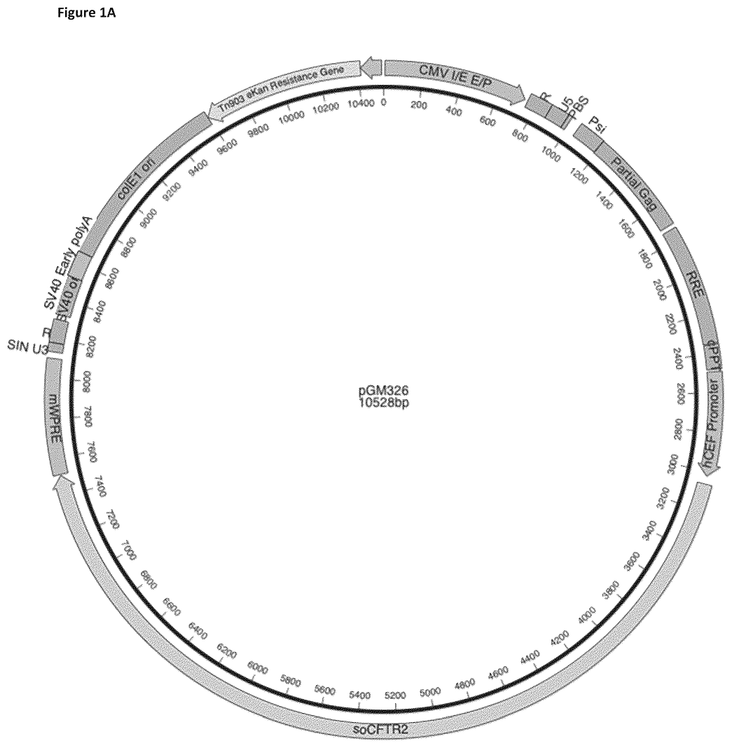

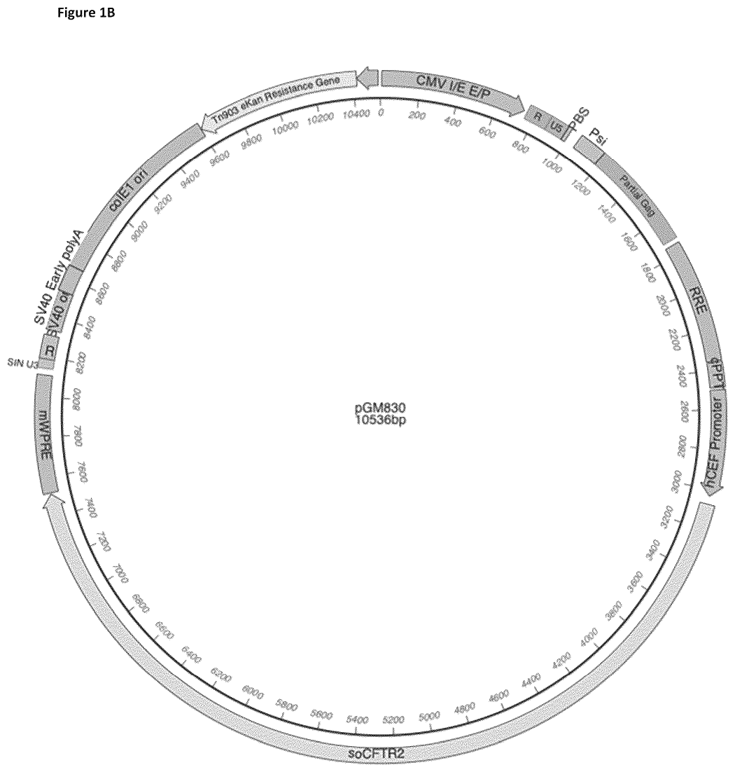

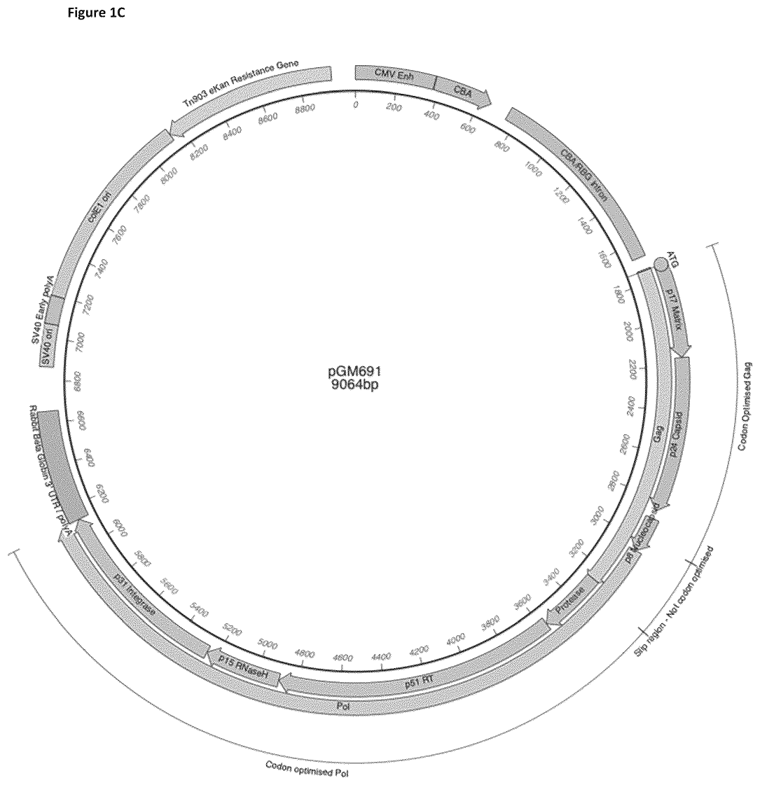

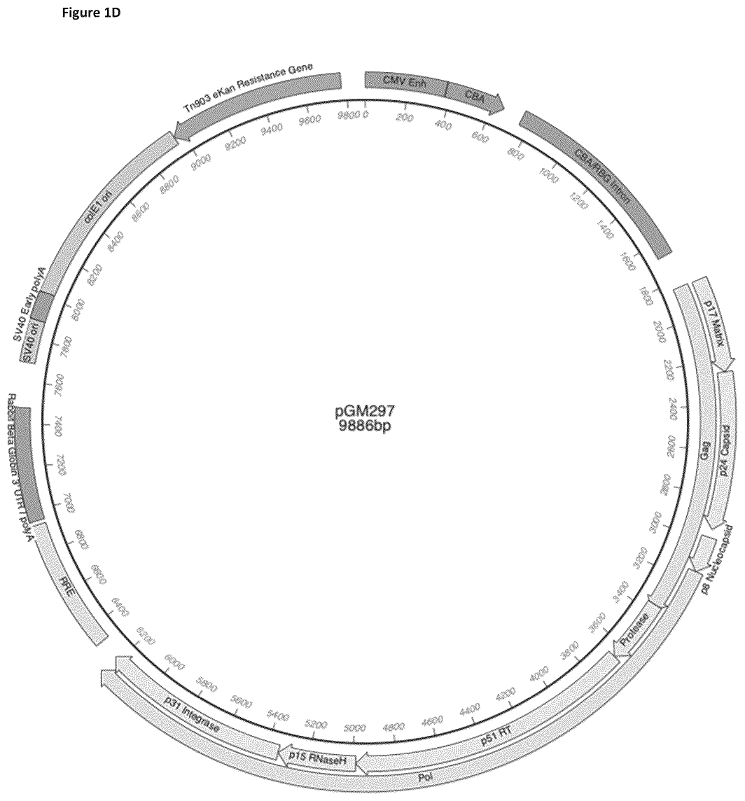

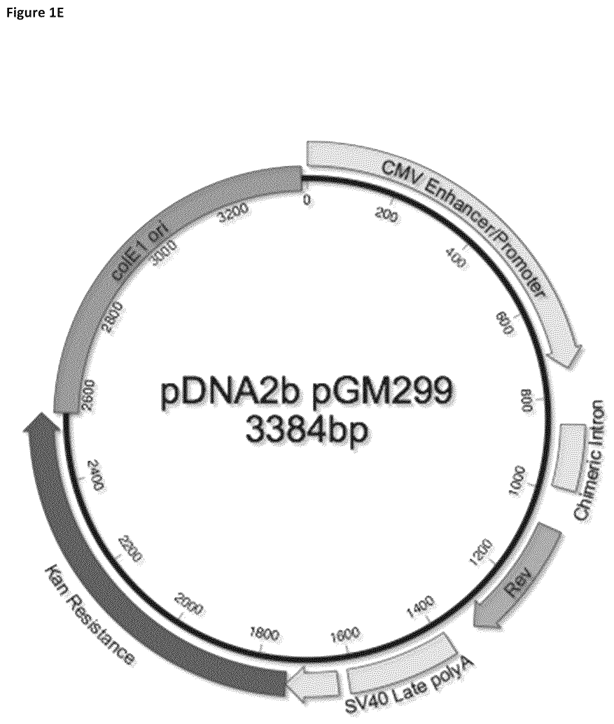

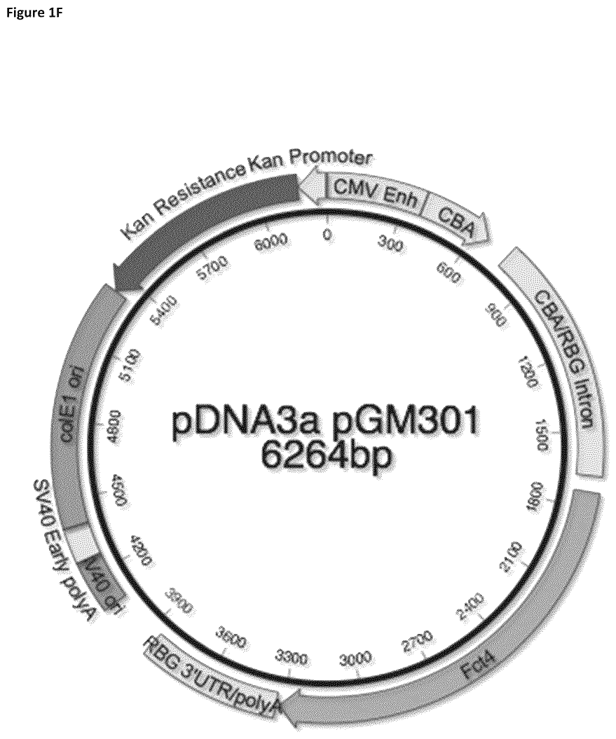

The retroviral/lentiviral (e.g. SIV) vector of the invention may have no intron positioned between the promoter and the transgene. Similarly, there may be no intron between the promoter and the transgene in the vector genome (pDNA1) plasmid (for example, pGM326 as described herein, illustrated in FIG. 1A and with the sequence of SEQ ID NO: 3).

Preferably, the retroviral/lentiviral (e.g. SIV) vector comprises a hCEF promoter and a CFTR transgene, including those described herein. Optionally said retroviral/lentiviral (e.g. SIV) vector may have no intron positioned between the promoter and the transgene. Such a retroviral/lentiviral (e.g. SIV) vector may be produced by the method described herein, using a genome plasmid carrying the CFTR transgene and a promoter. Particularly preferred is a SIV.F/HN vector with a hCEF promoter and a CFTR transgene, including those described herein.

The retroviral/lentiviral (e.g. SIV) vector as described herein comprises at least one transgene. The transgene comprises a nucleic acid sequence encoding a gene product, e.g., a protein, particularly a therapeutic protein, preferably said at least one transgene comprises or consists of CFTR.

For example, the nucleic acid sequence encoding a CFTR comprises (or consists of) a nucleic acid sequence having at least 90% (such as at least 90, 92, 94, 95, 96, 97, 98, 99 or 100%) sequence identity to the CFTR nucleic acid sequence respectively, examples of which are described herein. In a further embodiment, the nucleic acid sequence encoding CFTR comprises (or consists of) a nucleic acid sequence having at least 95% (such as at least 95, 96, 97, 98, 99 or 100%) sequence identity to the CFTR nucleic acid sequence respectively, examples of which are described herein. In one embodiment, the nucleic acid sequence encoding CFTR is provided by SEQ ID NO: 1, or variants thereof.

The amino acid sequence of the CFTR encoded by the CFTR transgene may comprise (or consist of) an amino acid sequence having at least 95% (such as at least 95, 96, 97, 98, 99 or 100%) sequence identity to the functional CFTR polypeptide sequence respectively.

The retroviral/lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO: 14.

The retroviral/lentiviral (e.g. SIV) vectors according to the invention may be as described in WO 2015/177501, International Application No. PCT/GB2022/050524 (which claims priority from UK Patent Application No. 2102832.9) and UK Patent Application No. 2212472.1, each of which is herein incorporated by reference in its entirety. Particularly preferred is a retroviral/lentiviral (e.g. SIV) vectors according to UK Patent Application No. 2212472.1.

Thus, particularly preferred is a retroviral/lentiviral (e.g. SIV) vector which is an SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 16 (which comprises a CFTR transgene), preferably wherein the modified retroviral RNA sequence consists of a nucleic acid sequence of SEQ ID NO: 16; and (b) the F protein comprises a first subunit which comprises or consists of an amino acid sequence of SEQ ID NO: 19 and a second subunit which comprises or consists of an amino acid sequence of SEQ ID NO: 20. Said vector may further comprise one or more of: (a) a p17 protein comprising or consisting of an amino acid sequence of SEQ ID NO: 22; (b) a p24 protein comprising or consisting of an amino acid sequence of SEQ ID NO: 23; (c) p8 protein comprising or consisting of an amino acid sequence of SEQ ID NO: 24; (d) a protease comprising or consisting of an amino acid sequence of SEQ ID NO: 25; (e) a p51 protein comprising or consisting of an amino acid sequence of SEQ ID NO: 26; (f) a p15 protein comprising or consisting of an amino acid sequence of SEQ ID NO: 27; (g) a p31 protein comprising or consisting of an amino acid sequence of SEQ ID NO: 28; (h) a Gag protein comprising or consisting of an amino acid sequence of SEQ ID NO: 29; and/or (i) a Pol protein comprising or consisting of an amino acid sequence of SEQ ID NO: 30; wherein optionally the vector comprises each of (a) to (g).

Methods of Making Retroviral/Lentiviral (e.g. SIV) Vectors

The retroviral/lentiviral (e.g. SIV) vectors of the invention may be produced by any appropriate method. Non-limiting examples of such methods are described in WO 2015/177501, International Application No. PCT/GB2022/050524 (which claims priority from UK Patent Application No. 2102832.9) and UK Patent Application No. 2212472.1, each of which is herein incorporated by reference in its entirety. Particularly preferred is a method as described in UK Patent Application No. 2212472.1.

The retroviral/lentiviral (e.g. SIV) vectors of the invention are typically produced by a scalable GMP-compatible method. Exemplary methods are described herein. The present invention encompasses combination therapies comprising the use of retroviral/lentiviral (e.g. SIV) vectors, particularly SIV.F/HN vectors obtained or obtainable by any method described herein.

The production of retroviral/lentiviral (e.g. SIV) vectors typically employs one or more plasmids which provide the elements needed for the production of the vector: the genome for the retroviral/lentiviral vector, the Gag-Pol, Rev, F and HN. Multiple elements can be provided on a single plasmid. Preferably each element is provided on a separate plasmid, such that there are five plasmids, one for each of the vector genome, the Gag-Pol, Rev, F and HN, respectively.

Alternatively, a single plasmid may provide the Gag-Pol and Rev elements, and may be referred to as a packaging plasmid (pDNA2). The remaining elements (genome, F and HN) may be provided by separate plasmids (pDNA1, pDNA3a, pDNA3b respectively), such that four plasmids are used for the production of a retroviral/lentiviral (e.g. SIV) vector according to the invention. In the four plasmid methods, pDNA1, pDNA3a and pDNA3b may be as described herein in the context of the five-plasmid method.

Any one of the plasmids used in the production of a retroviral/lentiviral (e.g. SIV) vectors of the invention may independently be codon optimised or at least partially codon optimised. Partial codon-optimisation encompasses at least 50%, at least 60%, at least 70%, at least 80%, at least 95%, at least 90%, at least 95%, at least 96%, at least 97%, at least 98%, at least 99% or more codon optimisation. Codon optimisation is a technique to maximise protein expression by increasing the translational efficiency of the encoding gene. Translational efficiency is increased by modification of the nucleic acid sequence. Codon optimisation is routine in the art, and it is within the routine practice of one of ordinary skill to devise a codon-optimised version of a given nucleic acid sequence. As described herein, the transgene and/or promoter may each independently be codon-optimised. Alternatively or in addition, the Gag-Pol genes may be codon-optimised. In particular, codon-optimisation of the Gag-Pol genes is preferred as can improve the safety profile of the resulting retroviral/lentiviral (e.g. SIV) vectors, particularly SIV.F/HN vectors, without negatively impacting the vector titre, and can even increase vector titre (as described in International Application No. PCT/GB2022/050524, which claims priority from UK Patent Application No. 2102832.9).

Accordingly, the retroviral/lentiviral (e.g. SIV) vectors of the invention, particularly those pseudotyped with hemagglutinin-neuraminidase (HN) and fusion (F) proteins from a respiratory paramyxovirus, and comprising a promoter and a transgene, are produced by a method which comprises the use of codon-optimised gag-pol genes. Preferably the codon-optimised gag-pol genes used in the production methods of the invention are SIV gag-pol genes. Exemplary wild-type SIV gag-pol genes that may be modified to produce codon-optimised gag-pol genes are given in SEQ ID NO: 5. Exemplary codon-optimised gag-pol genes derived from SEQ ID NO: 5 are given in SEQ ID NO: 6. In addition to codon-optimisation, the codon-optimised gag-pol genes used in the production methods of the invention may comprise other modifications, such as a translational slip (which allows translation to slip from one region to another to allow the production of both Gag and Pol). Any suitable variation of codon usage may be used in the codon-optimised gag-pol genes of the invention, provided that (i) homology between the vector genome plasmid and GagPol plasmid is reduced to minimise the risk of RCL production and (ii) after codon optimisation there is production of sufficient GagPol without the inclusion of RRE (this further reduces homology and the risk of RCL production).

The codon-optimised gag-pol genes used in the production methods of the invention may be completely (100%) or partially codon-optimised. Partial codon-optimisation of the gag-pol genes encompasses at least 50%, at least 60%, at least 70%, at least 80%, at least 95%, at least 90%, at least 95%, at least 96%, at least 97%, at least 98%, at least 99% or more codon optimisation.

Preferably, the gag-pol genes themselves are completely codon-optimised, but may comprise non-contain regions of non-codon-optimised sequence (e.g. between the gag and pol genes). By way of non-limiting example, to maintain the translational slip of reading frames between the gag and pol genes, the region around the translational slip sequence may not be codon-optimised (e.g. in case the precise translational slip sequence is important for this function). A non-codon-optimised translational slip sequence within codon-optimised gag-pol genes is exemplified in SEQ ID NO: 6.

Preferably, the codon-optimised gag-pol genes used to produce a retroviral/lentiviral (e.g. SIV) of the invention comprise or consist of the nucleic acid sequence of SEQ ID NO: 6, or a variant thereof (as defined herein). In particular, the codon-optimised gag-pol genes may comprise or consist of a nucleic acid sequence having at least 80%, at least 85%, at least 90%, at least 95%, at least 96%, at least 97%, at least 98%, at least 99% or more sequence identity to SEQ ID NO: 6. Preferably, the codon-optimised gag-pol genes used in a method of the invention comprise or consist of a nucleic acid sequence having at least 90%, more preferably at least 95%, even more preferably at least 98%, or more sequence identity to SEQ ID NO: 6. The codon-optimised gag-pol genes of SEQ ID NO: 6 comprise a translational slip, and so do not form a single conventional open reading frame.

Preferably, the codon-optimised gag-pol genes used in a method of the invention are comprised in a plasmid that comprises or consists of a nucleic acid sequence of SEQ ID NO: 7 (pGM691), or a variant thereof (as defined herein). In particular, the codon-optimised gag-pol genes are comprised in a plasmid that comprises or consists of a nucleic acid sequence having at least 80%, at least 85%, at least 90%, at least 95%, at least 96%, at least 97%, at least 98%, at least 99% or more sequence identity to SEQ ID NO: 7. Preferably, the codon-optimised gag-pol genes are comprised in a plasmid that comprises or consists of a nucleic acid sequence having at least 90%, more preferably at least 95%, even more preferably at least 98%, or more sequence identity to SEQ ID NO: 7. In the plasmid of SEQ ID NO: 7 (or variants thereof): (i) the codon-optimised gag-pol genes of SEQ ID NO: 6 comprise a translational slip, and so do not form a single conventional open reading frame; and (ii) the codon-optimised gag-pol genes of SEQ ID NO: 6 are operably linked to a CAG promoter. An exemplary CAG promoter is set out in SEQ ID NO: 15.

In the preferred five plasmid method of the invention, the vector genome plasmid encodes all the genetic material that is packaged into final retroviral/lentiviral vector, including the transgene. Typically only a portion of the genetic material found in the vector genome plasmid ends up in the virus. The vector genome plasmid may be designated herein as “pDNA1”, and typically comprises the transgene and the transgene promoter.

The other four plasmids are manufacturing plasmids encoding the Gag-Pol, Rev, F and HN proteins. These plasmids may be designated “pDNA2a”, “pDNA2b”, “pDNA3a” and “pDNA3b” respectively.

Modifications may be made to the vector genome plasmid (pDNA1), particularly to further improve the safety profile of the vector. As exemplified herein, such modifications may comprise or consist of modifying the pDNA1 sequence to remove viral, particularly retroviral/lentiviral (e.g. SIV), ORFs from the pDNA1 sequence. Thus, the retroviral/lentiviral (e.g. SIV) vectors of the invention may be made using a modified pDNA1 which comprises a reduced number of non-transgene ORFs. Said modified pDNA1 may comprise modifications within any region of the plasmid sequence. In particular, a modified pDNA1 may comprise modifications to remove: (i) 5′ to 3′ ORFs; (ii) ORFs of 100 amino acids; and/or (iii) ORFs upstream of the transgene and/or the promoter operably linked to the transgene. Whilst a modified pDNA1 may comprise no ORFs other than the transgene, this is not essential. Rather, a modified pDNA1 may still comprise ORFs other than the transgene, but may comprise a reduced number of non-transgene ORFs compared to the unmodified pDNA1 from which it is derived. By way of non-limiting example, a modified pDNA1 may comprise at least 1, at least 2, at least 3, at least 4, at least 5 or more fewer non-transgene ORFs compared with the corresponding unmodified pDNA1. As a specific example, pGM830 (which is derived from pGM326) comprises 2 fewer non-transgene ORFs compared with pGM326. A modified pDNA1 may comprise at least 1, at least 5, at least 6, at least 7, at least 8, at least 9, at least 10, at least 15, at least 20, or more modifications (e.g. 1, 2, 3, 4, 5, 6, 7, 8, 9, 10, 15 or 20 modifications) compared with the corresponding unmodified pDNA1. By way of non-limiting example, a modified pDNA1 may comprise between about 1 to about 20, such as between about 5 to about 15, or between about 5 to about 10 modifications compared with the corresponding unmodified pDNA1. As a specific example, pGM830 (which is derived from pGM326) comprises 7 modifications compared with pGM326.

The use of modified pDNA1 (e.g. pGM830) as described herein has the potential to produce an improved SIV titre compared with a production method which uses an unmodified pDNA1 plasmid (e.g. pGM326), but in which all other plasmids and method parameters are kept constant.

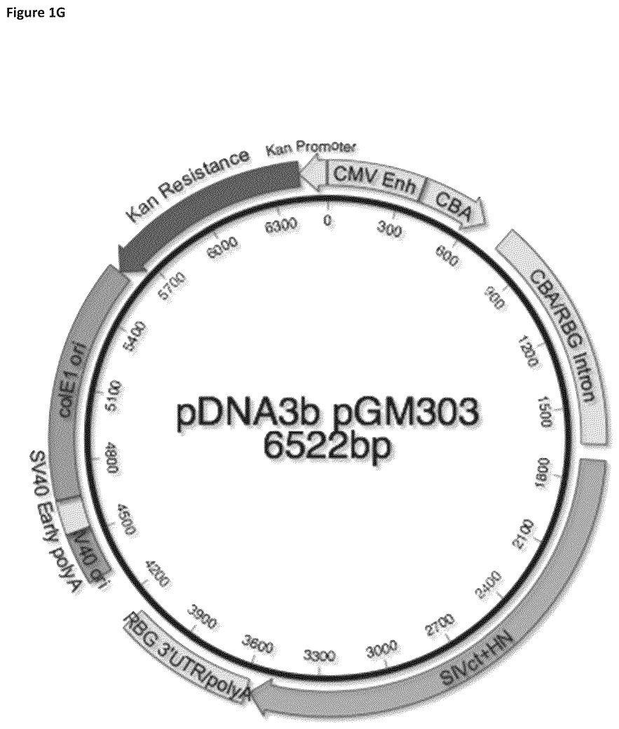

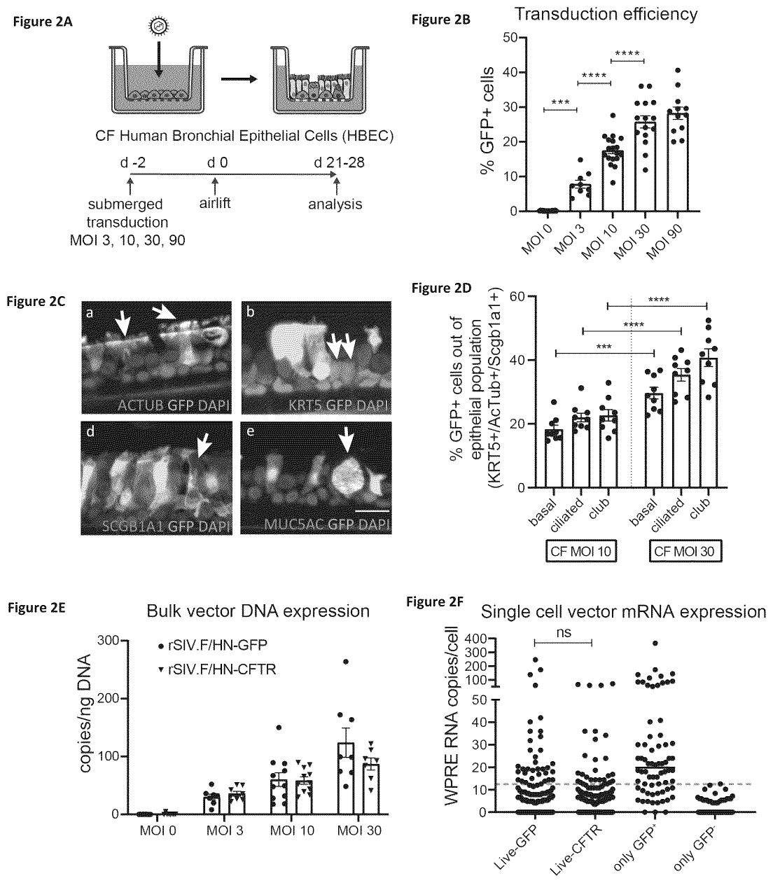

The five plasmids may be characterised by FIGS. 1A-2G, thus pDNA1 is the pGM326 plasmid of FIG. 1A or the pGM830 plasmid of FIG. 1B, pDNA2a is the pGM691 plasmid of FIG. 1C or the pGM297 of FIG. 1D, pDNA2b is the pGM299 plasmid of FIG. 1E, pDNA3a is the pGM301 plasmid of FIG. 1F and pDNA3b is the pGM303 plasmid of FIG. 1G, or variants thereof any of these plasmids (as described herein). Using the plasmids pGM326, pGM297, pGM299, pGM301 and pGM303, the final CFTR containing retroviral/lentiviral vector may be referred to as vGM058. Using the plasmids pGM326, pGM691, pGM299, pGM301 and pGM303, the final CFTR containing retroviral/lentiviral vector may be referred to as vGM195. The pGM691 plasmid (typically with pGM326, pGM299, pGM301 and pGM303) and the vGM195 vector may be preferred. Using the plasmids pGM830, pGM691, pGM299, pGM301 and pGM303, the final CFTR containing retroviral/lentiviral vector may be referred to as vGM244. The pGM691 and pGM830 plasmids (typically with pGM299, pGM301 and pGM303) and the vGM244 vector may be particularly preferred.

The pGM326 plasmid as defined in FIG. 1A is represented by SEQ ID NO: 8; the pGM830 plasmid as defined in FIG. 13 is represented by SEQ ID NO: 9; the pGM691 plasmid as defined in FIG. 1C is represented by SEQ ID NO: 7; the pGM297 plasmid as defined in FIG. 1D is represented by SEQ ID NO: 10; the pGM299 plasmid as defined in FIG. 1E is represented by SEQ ID NO: 11; the pGM301 plasmid as defined in FIG. 1F is represented by SEQ ID NO: 12; and the pGM303 plasmid as defined in FIG. 1G is represented by SEQ ID NO: 13. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 7 to 13 are encompassed.

In the five-plasmid method of the invention all five plasmids contribute to the formation of the final retroviral/lentiviral (e.g. SIV) vector. During manufacture of the retroviral/lentiviral (e.g. SIV) vector, the vector genome plasmid (pDNA1) provides the enhancer/promoter, Psi, RRE, cPPT, mWPRE, SIN LTR, SV40 polyA (see FIG. 1A or 1B), which are important for virus manufacture. Using pGM326 or pGM830 as non-limiting examples of a pDNA1, the CMV enhancer/promoter, SV40 polyA, colE1 Ori and KanR are involved in manufacture of the retroviral/lentiviral (e.g. SIV) vector of the invention (e.g. vGM195 or vGM244), but are not found in the final retroviral/lentiviral (e.g. SIV) vector. The RRE, cPPT (central polypurine tract), hCEF, soCFTR2 (transgene) and mWPRE from pGM326 or pGM830 are found in the final retroviral/lentiviral (e.g. SIV) vector. SIN LTR (long terminal repeats, SIN/IN self-inactivating) and Psi (packaging signal) may be found in the final retroviral/lentiviral (e.g. SIV) vector.

For other retroviral/lentiviral (e.g. SIV) vectors of the invention, corresponding elements from the other vector genome plasmids (pDNA1) are required for manufacture (but not found in the final vector), or are present in the final retroviral/lentiviral (e.g. SIV) vector.

The F and HN proteins from pDNA3a and pDNA3b (preferably Sendai F and HN proteins) are important for infection of target cells with the final retroviral/lentiviral (e.g. SIV) vector, i.e. for entry of a patient's epithelial cells (typically lung, preferably airway epithelial cells, as described herein). The products of the pDNA2a and pDNA2b plasmids are important for virus transduction, i.e. for inserting the retroviral/lentiviral (e.g. SIV) DNA into the host's genome. The promoter, regulatory elements (such as WPRE) and transgene are important for transgene expression within the target cell(s).

A retroviral/lentiviral (e.g. SIV) vectors of the invention may be produced by a method comprising or consisting of the following steps: (a) growing cells in suspension; (b) transfecting the cells with one or more plasmids; (c) adding a nuclease; (d) harvesting the lentivirus (e.g. SIV); (e) adding trypsin; and (f) purification of the lentivirus (e.g. SIV).

This method may use the four- or five-plasmid system described herein. Thus, for the preferred five-plasmid method, the one or more plasmids may comprise or consist of: a vector genome plasmid pDNA1; a gag-pol plasmid, pDNA2a; a Rev plasmid, pDNA2b; a fusion (F) protein plasmid, pDNA3a; and a hemagglutinin-neuraminidase (HN) plasmid, pDNA3b. The pDNA1 may be selected from pGM326 and pGM830, preferably pGM830. The pDNA2a may be selected from pGM297 and pGM691, preferably pGM297. The pDNA2b may be pGM299. The pDNA3a may be pGM301. The pDNA3b may be pGM303. Any combination of pDNA1, pDNA2a, pDNA2b, pDNA3a and pDNA3b may be used. Preferably, the pDNA1 is pGM326 or pGM830 (pGM830 being particularly preferred); the pDNA2a is pGM297 or pGM691 (pGM691 being particularly preferred); the pDNA2b is pGM299; the pDNA3a is pGM301; and the pDNA3b is pGM303. A SIV vector produced using pGM830, pGM691, pGM299, pGM301, and pGM303 is designated vGM244. A SIV vector produced using pGM326, pGM691, pGM299, pGM301, and pGM303 is designated vGM195. vGM195 and vGM244 are preferred SIV.F/HN vectors for use in combination therapies according to the invention, with vGM244 being particularly preferred.

Any appropriate ratio of vector genome plasmid:co-gagpol plasmid:Rev plasmid:F plasmid:HN plasmid may be used to in the production of a retroviral/lentiviral (e.g. SIV).

Steps (a)-(f) of the method are typically carried out sequentially, starting at step (a) and continuing through to step (f). The method may include one or more additional step, such as additional purification steps, buffer exchange, concentration of the retroviral/lentiviral (e.g. SIV) vector after purification, and/or formulation of the retroviral/lentiviral (e.g. SIV) vector after purification (or concentration). Each of the steps may comprise one or more sub-steps. For example, harvesting may involve one or more steps or sub-steps, and/or purification may involve one or more steps or sub-steps.

Any appropriate cell type may be transfected with the one or more plasmids (e.g. the five-plasmids described herein) to produce a retroviral/lentiviral (e.g. SIV) vector of the invention. Typically mammalian cells, particularly human cell lines are used. Non-limiting examples of cells suitable for use in the methods of the invention are HEK293 cells (such as HEK293F or HEK293T cells) and 293T/17 cells. Commercial cell lines suitable for the production of virus are also readily available (e.g. Gibco Viral Production Cells—Catalogue Number A35347 from ThermoFisher Scientific).

The cells may be grown as adherent or suspension culture in animal-component free media, including serum-free media. The cells may be grown in a media which contains human components. The cells may be grown in a defined media comprising or consisting of synthetically produced components.

Any appropriate transfection means may be used according to the invention. Selection of appropriate transfection means is within the routine practice of one of ordinary skill in the art. By way of non-limiting example, transfection may be carried out by the use of PEIPro™, Lipofectamine2000™, Lipofectamine3000 ™ or calcium triphosphate.

Any appropriate nuclease may be used according to the invention. Selection of appropriate nuclease is within the routine practice of one of ordinary skill in the art. Typically the nuclease is an endonuclease. By way of non-limiting example, the nuclease may be Benzonase® or Denarase®. The addition of the nuclease may be at the pre-harvest stage or at the post-harvest stage, or between harvesting steps.

The trypsin activity may preferably be provided by an animal origin free, recombinant enzyme such as TrypLE Select™. The addition of trypsin may be at the pre-harvest stage or at the post-harvest stage, or between harvesting steps.

Any appropriate purification means may be used to purify the retroviral/lentiviral (e.g. SIV) vector. Non-limiting examples of suitable purification steps include depth/end filtration, tangential flow filtration (TFF) and chromatography. The purification step typically comprises at least one chromatography step. Non-limiting examples of chromatography steps that may be used in accordance with the invention include mixed-mode size exclusion chromatography (SEC) and/or anion exchange chromatography. Elution may be carried out with or without the use of a salt gradient, preferably without.

This method may be used to produce the retroviral/lentiviral (e.g. SIV) vectors of the invention as described herein. Alternatively, the retroviral/lentiviral (e.g. SIV) vector of the invention comprises any of the above-mentioned genes, or the genes encoding the above-mentioned proteins.

A retroviral/lentiviral (e.g. SIV) vector of the invention may be produced by a method using any combination of one or more of the specific plasmid constructs provided by FIGS. 1A-1G.

### CFTR Mutations

CF is caused by mutations in the CFTR gene. To-date, over 2000 different mutations have been identified within the CFTR gene. Some CFTR gene mutations result in no CFTR protein being produced. Others result in the production of a dysfunctional CFTR protein. Using the current conventional nomenclature, the different CF-causing mutations in the CFTR gene can be arranged into classes depending on the effect of the mutation on CFTR protein production, conformation or function.

Class I CFTR mutations are protein production mutations, which result in no functional CFTR protein being produced. Approximately 22% of CF patients have at least one class I CFTR mutation. Several nonsense and splice mutations fall within class I. Examples of class I CFTR mutations include G542X, W1282X and R553X.

Class II CFTR mutations are protein processing mutations. Class II CFTR mutations do not prevent CFTR protein being produced, but the translated CFTR protein is misfolded and cannot form the correct conformation. Typically CFTR protein with a class II mutation will not be transported to the cell membrane, or is transported at reduced levels compared with normal CFTR protein. Approximately 88% of CF patients have at least one class II CFTR mutation. Examples of class II CFTR mutations include F508del, N1303K and I507del. F508del is the most common CF-causing CFTR mutation

Class III CFTR mutations are gating mutations. Class III CFTR mutations do not prevent CFTR protein being produced or transported to the cell membrane. Rather, gating mutations force the CFTR protein to adopt a closed conformation, preventing or reducing chloride transport. Approximately 6% of CF patients have at least one class III CFTR mutation. Examples of class III CFTR mutations include G551D and S549N.

Class IV CFTR mutations are conduction mutations. Class IV CFTR mutations do not prevent CFTR protein being produced or transported to the cell membrane, nor do they hold the CFTR protein in a closed conformation. However, class IV mutations can affect the inner conformation of the chloride channel within the CFTR protein, reducing chloride transport. Approximately 6% of CF patients have at least one class IV CFTR mutation. Examples of class IV CFTR mutations include D1152H, R347P and R117H.

Class V CFTR mutations are termed “insufficient protein mutations”. Class V CFTR mutations result in a lower amount of CTFR protein being present at the cell membrane. This may occur because less CFTR protein is produced, only a small number of protein at the cell surface work correctly, or degradation of normal CFTR protein in the cell membrane occurs too quickly. Several missense and splice mutations fall within class V. Approximately 5% of CF patients have at least one class V CFTR mutation. Examples of class V CFTR mutations include 3849+10kbC→T, 2789+5G→A and A455E.

Class VI CFTR mutations destabilise the CFTR protein in post-endoplasmic reticulum (ER) compartments and/or at the cell membrane, by reducing conformational stability of the CFTR and/or by generating additional internalisation signals. These mutations consequently result in accelerated CFTR turnover at the cell membrane and reduced expression at the apical cell membrane.

The present invention relates to the treatment of CF caused by any combination of class I, II, III, IV, V and/or VI mutations. A patient to be treated according to the present invention may have CF caused by one or more class I mutation, one or more class II mutation, one or more class III mutation, one or more class IV mutation, one or more class V mutation and/or one or more class VI mutation2. A patient to be treated may have (i) one or more mutation in class I and one or more mutation in class II; (ii) one or more mutation in class I and one or more mutation in class III; (iii) one or more mutation in class I and one or more mutation in class IV; (iv) one or more mutation in class I and one or more mutation in class V; (v) one or more mutation in class I and one or more mutation in class VI; (vi) one or more mutation in class II and one or more mutation in class III; (vii) one or more mutation in class II and one or more mutation in class IV; (viii) one or more mutation in class II and one or more mutation in class V; (ix) one or more mutation in class II and one or more mutation in class VI; (x) one or more mutation in class III and one or more mutation in class IV; (xi) one or more mutation in class III and one or more mutation in class V; (xii) one or more mutation in class III and one or more mutation in class VI; (xiii) one or more mutation in class IV and one or more mutation in class V; (xiv) one or more mutation in class IV and one or more mutation in class VI; (xv) one or more mutation in class V and one or more mutation in class VI; (xvi) one or more mutation in class I, one or more mutation in class II and one or more mutation in class III; (xvii) one or more mutation in class I, one or more mutation in class II and one or more mutation in class IV; (xviii) one or more mutation in class I, one or more mutation in class II and one or more mutation in class V; (xix) one or more mutation in class I, one or more mutation in class II and one or more mutation in class VI; (xx) one or more mutation in class I, one or more mutation in class III and one or more mutation in class IV; (xxi) one or more mutation in class I, one or more mutation in class III and one or more mutation in class V; (xxii) one or more mutation in class I, one or more mutation in class III and one or more mutation in class VI; (xxiii) one or more mutation in class I, one or more mutation in class IV and one or more mutation in class V; (xxiv) one or more mutation in class I, one or more mutation in class IV and one or more mutation in class VI; (xxv) one or more mutation in class I, one or more mutation in class V and one or more mutation in class VI; (xxvi) one or more mutation in class II, one or more mutation in class III and one or more mutation in class IV; (xxvii) one or more mutation in class II, one or more mutation in class III and one or more mutation in class V; (xxviii) one or more mutation in class II, one or more mutation in class III and one or more mutation in class VI; (xxix) one or more mutation in class II, one or more mutation in class IV and one or more mutation in class V; (xxx) one or more mutation in class II, one or more mutation in class IV and one or more mutation in class VI; (xxxi) one or more mutation in class II, one or more mutation in class V and one or more mutation in class VI; (xxxii) one or more mutation in class III, one or more mutation in class IV and one or more mutation in class V; (xxxiii) one or more mutation in class III, one or more mutation in class IV and one or more mutation in class VI; (xxxiv) one or more mutation in class III, one or more mutation in class V and one or more mutation in class VI; (xxxv) one or more mutation in class IV, one or more mutation in class V and one or more mutation in class VI; (xxxvi) one or more mutation in class I, one or more mutation in class II, one or more mutation in class III and one or more mutation in class IV; (xxxvii) one or more mutation in class I, one or more mutation in class II, one or more mutation in class III and one or more mutation in class V; (xxxviii) one or more mutation in class I, one or more mutation in class II, one or more mutation in class III and one or more mutation in class VI; (xxxix) one or more mutation in class I, one or more mutation in class II, one or more mutation in class IV and one or more mutation in class V; (xl) one or more mutation in class I, one or more mutation in class II, one or more mutation in class IV and one or more mutation in class VI; (xli) one or more mutation in class I, one or more mutation in class II, one or more mutation in class V and one or more mutation in class VI; (xlii) one or more mutation in class I, one or more mutation in class III, one or more mutation in class IV and one or more mutation in class V; (xliii) one or more mutation in class I, one or more mutation in class III, one or more mutation in class IV and one or more mutation in class VI; (xliv) one or more mutation in class I, one or more mutation in class III, one or more mutation in class V and one or more mutation in class VI; (xlv) one or more mutation in class I, one or more mutation in class IV, one or more mutation in class V and one or more mutation in class VI; (xlvi) one or more mutation in class II, one or more mutation in class III, one or more mutation in class IV and one or more mutation in class V; (xlvii) one or more mutation in class II, one or more mutation in class III, one or more mutation in class IV and one or more mutation in class VI; (xlviii) one or more mutation in class II, one or more mutation in class III, one or more mutation in class V and one or more mutation in class VI; (xlix) one or more mutation in class II, one or more mutation in class IV, one or more mutation in class V and one or more mutation in class VI; (I) one or more mutation in class III, one or more mutation in class IV, one or more mutation in class V and one or more mutation in class VI; (Ii) one or more mutation in class I, one or more mutation in class II, one or more mutation in class III, one or more mutation in class IV and one or more mutation in class V; (hi) one or more mutation in class I, one or more mutation in class II, one or more mutation in class III, one or more mutation in class IV and one or more mutation in class VI; (liii) one or more mutation in class I, one or more mutation in class II, one or more mutation in class III, one or more mutation in class V and one or more mutation in class VI; (liv) one or more mutation in class I, one or more mutation in class II, one or more mutation in class IV, one or more mutation in class V and one or more mutation in class VI; (Iv) one or more mutation in class I, one or more mutation in class III, one or more mutation in class IV, one or more mutation in class V and one or more mutation in class VI; (Ivi) one or more mutation in class II, one or more mutation in class III, one or more mutation in class IV, one or more mutation in class V and one or more mutation in class VI; or (Ivii) one or more mutation in class I, one or more mutation in class II, one or more mutation in class III, one or more mutation in class IV, one or more mutation in class V and one or more mutation in class VI.

A patient to be treated according to the invention may have at least 1, at least 2, at least 3, at least 4, at least 5, at least 6 at least 7, at least 8, at least 9, at least 10 or more mutations in the CFTR gene, which may each be independently selected from a class I, class II, class III, class IV and/or class V mutation as described herein.

A patient to be treated according to the invention may have at least one class I and/or class II CFTR mutation, such as those described herein. A patient to be treated may have (i) at least one class I CFTR mutation that is optionally selected from G542X, W1282X and/or R553C; and/or (ii) at least one class II CFTR mutation that is optionally selected from F508del, N1303K and/or I507del.

As exemplified herein, a combination treatment of the invention successfully restored CFTR expression and function in a model of a class I CFTR mutation, and that the surprising effect of the combination was greater on the CFTR transgene expressed by the retro/lentiviral (e.g. SIV) vector than the endogenous CFTR gene. Accordingly, the combination therapy of the invention may be suitable for use independent of the CFTR mutation of the patient. In other words, and without being bound by theory, as the CFTR modulator may exert a greater therapeutic effect on the CFTR transgene, the nature of the CF-causing mutation in the endogenous CFTR gene may be irrelevant. The present invention therefore has the potential to treat patients with CF independent of the CF-causing mutation. This is advantageous, as the currently authorised CFTR modulator therapies are suitable only for patients with specific CFTR mutations.

### CFTR Modulators

CFTR modulators are active pharmaceutical ingredients (APIs) designed to correct malfunctioning CFTR proteins. CFTR modulators are a specialised group of therapies, as different modulators are designed to address the underlying defect in the CFTR protein caused by a specific CFTR mutation or class of CFTR mutations.

There are three main types of CFTR modulators: CFTR potentiators, CFTR correctors and CFTR amplifiers.

As discussed herein, class III CFTR mutations, such as G551D, are gating mutations, which prevent normal opening of the CFTR protein to facilitate chloride transport. CFTR potentiators mitigate this defect by opening the CFTR protein gates and keeping them open longer to facilitate the smooth flow of chloride ions. Ivacaftor (Kalydeco®) is an example of a CFTR potentiator developed by Vertex Pharmaceuticals. It is an oral medication approved by the U.S. Food and Drug Administration (FDA), the EU, and Health Canada for CF patients as young as 1 year with at least one mutation (such as G551D) that impairs chloride ion flow. Another example of a CFTR potentiator is the experimental treatment PTI-808 being developed by Proteostasis Therapeutics.

As discussed herein, class II CFTR mutations are protein processing mutations, which result in misfolding of the CFTR protein, which can affect transport of the misfolded CFTR to the cell surface. CFTR correctors assist the CFTR protein in folding correctly into its 3D conformation, allowing it to be successfully transported to the cell membrane so that it can function. Lumacaftor (VX-809) and tezacaftor (VX-661) are two therapies by Vertex Pharmaceuticals that function as correctors. Another CFTR corrector is elexacaftor. These correctors help the CFTR protein fold correctly and reach the cell surface, but fall short in alleviating CF symptoms by themselves. Therefore, they are not approved as a monotherapy for CF. Another CFTR corrector under development (by Proteostasis Therapeutics) is PTI-801.

As discussed herein, class V CFTR mutations result in reduced levels of CFTR protein being present at the cell surface, for example because they result in lower levels of CFTR protein being expressed, or increase the rate of degradation of CFTR protein. Amplifiers are a type of CFTR modulator that enhances the production of CFTR protein by the cells. PTI-428 is an investigational first-generation CFTR amplifier by Proteostasis Therapeutics, which is being tested as a single and combination therapy for CF.

CFTR potentiators, correctors and amplifiers are described in the art for use independently as single therapies. In addition, combinations of these CFTR modulators are also known, with three of the four currently approved CFTR modulator therapies being combination therapies. By way of non-limiting example, combining a potentiator with a corrector may improve CFTR activity by correcting the CFTR conformation and opening the gate to allow chloride transport. In particular, a combination of the potentiator ivacaftor and corrector lumacaftor is authorised as a combination therapy and is marketed as Orkambi® by Vertex for use in the treatment for CF patients with two F508del CFTR mutations. Another example of an authorised combination treatment is the potentiator ivacaftor and corrector tezacaftor, which is marked by Vertex as Symdeko® in the US and Symkevi® in the EU.

Amplifiers can also be used in combination with other CFTR modulators. Combining CFTR amplifiers with other CFTR modulators may be advantageous as the CFTR amplifier can result in more CFTR protein being expressed, which can then be acted upon by the other CFTR modulator(s).

In addition to these first-generation CFTR modulators, so-called next-generation modulators may combine multiple CFTR correctors to produce combination therapies with three or more APIs. An example of an approved next-generation CFTR modulator is Trikafta®, which is a combination of the potentiator ivacaftor and two correctors, tezacaftor and elexacaftor.

Any reference to a CFTR modulator herein encompasses any and all salts, derivatives and analogues of said CFTR modulator, unless expressly stated to the contrary. Thus, the invention relates to the combination of a known CFTR modulators such as those individualised herein (particularly ivacaftor) or a salt, derivative or analogue thereof. Salt forms preferably include pharmaceutically acceptable salts. Pharmaceutically acceptable salts include acid addition salts formed with inorganic acids such as, for example, hydrochloric or phosphoric acids, or with organic acids such as acetic, oxalic, tartaric, maleic, and the like. An “analogue” of a CFTR modulator, as the term is used herein, refers to a chemical structure that preserves substantial similarity with the CFTR modulator, although it may not be readily derived synthetically from the CFTR modulator. A related chemical structure that is readily derived synthetically from a CFTR modulator structure is referred to as a “derivative.” These are all within the scope of the invention. By way of non-limiting example, a reference to “ivacaftor” includes salt forms, analogues and derivatives of ivacaftor, such as deuterated ivacaftor (D-ivacaftor).

Accordingly, the invention relates to therapy comprising a gene therapy vector, particularly using a retro/lentiviral (e.g. SIV) vector as described herein, preferably a SIV.F/HN vector, in combination with one or more CFTR modulator, which may be selected from one or more CFTR potentiator, one or more CFTR corrector and/or one or more CFTR amplifier, or combination thereof. Combinations comprising one or more CFTR potentiator with a retro/lentiviral (e.g. SIV) vector are particularly preferred. As such, the invention may relate to the use of a gene therapy vector, particularly using a retro/lentiviral (e.g. SIV) vector as described herein, preferably a SIV.F/HN vector, in combination with one or more CFTR potentiator. One or more CFTR corrector and/or one or more CFTR amplifier may be used in addition to the combination of a gene therapy vector, particularly using a retro/lentiviral (e.g. SIV) vector as described herein, preferably a SIV.F/HN vector, and one or more CFTR potentiator.

Thus, the invention relates to a combination of a retro/lentiviral (e.g. SIV) vector as described herein, preferably a SIV.F/HN vector, and a CFTR modulator, which may be selected from one or more CFTR potentiator, one or more CFTR corrector and/or one or more CFTR amplifier, or combination thereof. Typically the invention relates to a combination of a retro/lentiviral (e.g. SIV) vector as described herein, preferably a SIV.F/HN vector, and a CFTR potentiator and/or CFTR corrector. Preferably, the invention relates to a combination of a retro/lentiviral (e.g. SIV) vector, as described herein, preferably a SIV.F/HN vector, and a CFTR potentiator, and optionally one or more CFTR corrector and/or one or more CFTR amplifier.

Any CFTR modulator may be used in combination with a retro/lentiviral (e.g. SIV) vector as described herein, preferably a SIV.F/HN vector, according to the present invention, such as those described herein. Accordingly, non-limiting examples of CFTR modulators that may be used in combination with a retro/lentiviral (e.g. SIV) vector as described herein, preferably a SIV.F/HN vector, according to the present invention include ivacaftor (Kalydeco®), PTI-808, VX-809 (Lumacaftor) and VX-661 (tezacaftor), elexacaftor, PTI-801, PTI-428, as well as combinations such as ivacaftor+lumacaftor (Orkambi®), ivacaftor+tezacaftor (Symdeko® or Symkevi®) and ivacaftor+tezacaftor+elexacaftor)(Trikafta®.

Preferably the invention relates to the combination of a retro/lentiviral (e.g. SIV) vector as described herein, preferably a SIV.F/HN vector, with a CFTR modulator selected from ivacaftor, tezacaftor, elexacaftor or lumacaftor, of a combination thereof. The combination of a retro/lentiviral (e.g. SIV) vector as described herein, preferably a SIV.F/HN vector, with the CFTR modulator (specifically the CFTR potentiator) ivacaftor is particularly preferred.

Preferred embodiments relate to the use of (A) a SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 16 (which comprises a CFTR transgene), preferably wherein the modified retroviral RNA sequence consists of a nucleic acid sequence of SEQ ID NO: 16; and (b) the F protein comprises a first subunit which comprises or consists of an amino acid sequence of SEQ ID NO: 19 and a second subunit which comprises or consists of an amino acid sequence of SEQ ID NO: 20 in combination with (B) a CFTR modulator selected from ivacaftor, tezacaftor, elexacaftor or lumacaftor, of a combination thereof.

Particularly preferred embodiments relate to the use of (A) a SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 16 (which comprises a CFTR transgene), preferably wherein the modified retroviral RNA sequence consists of a nucleic acid sequence of SEQ ID NO: 16; and (b) the F protein comprises a first subunit which comprises or consists of an amino acid sequence of SEQ ID NO: 19 and a second subunit which comprises or consists of an amino acid sequence of SEQ ID NO: 20 in combination with (B) the CFTR modulator (specifically the CFTR potentiator) ivacaftor.

As described herein, in said particularly preferred embodiments, the SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 16 (which comprises a CFTR transgene), preferably wherein the modified retroviral RNA sequence consists of a nucleic acid sequence of SEQ ID NO: 16; and (b) the F protein comprises a first subunit which comprises or consists of an amino acid sequence of SEQ ID NO: 19 and a second subunit which comprises or consists of an amino acid sequence of SEQ ID NO: 20, wherein said vector further comprise one or more of: (a) a p17 protein comprising or consisting of an amino acid sequence of SEQ ID NO: 22; (b) a p24 protein comprising or consisting of an amino acid sequence of SEQ ID NO: 23; (c) p8 protein comprising or consisting of an amino acid sequence of SEQ ID NO: 24; (d) a protease comprising or consisting of an amino acid sequence of SEQ ID NO: 25; (e) a p51 protein comprising or consisting of an amino acid sequence of SEQ ID NO: 26; (f) a p15 protein comprising or consisting of an amino acid sequence of SEQ ID NO: 27; (g) a p31 protein comprising or consisting of an amino acid sequence of SEQ ID NO: 28; (h) a Gag protein comprising or consisting of an amino acid sequence of SEQ ID NO: 29; and/or (i) a Pol protein comprising or consisting of an amino acid sequence of SEQ ID NO: 30; wherein optionally the vector comprises each of (a) to (g), and is combined with a CFTR modulator selected from ivacaftor, tezacaftor, elexacaftor or lumacaftor, of a combination thereof, particularly combined with the CFTR modulator (specifically the CFTR potentiator) ivacaftor.

### Therapeutic Indications

The retroviral/lentiviral (e.g. SIV) vectors of the present invention enable higher and sustained gene expression through efficient gene transfer. The F/HN-pseudotyped retroviral/lentiviral (e.g. SIV) vectors of the invention are capable of: (i) airway transduction without disruption of epithelial integrity; (ii) persistent gene expression; (iii) lack of chronic toxicity; and (iv) efficient repeated administration. Long term/persistent stable gene expression, preferably at a therapeutically-effective level, may be achieved using repeated doses of a vector of the present invention. Alternatively, a single dose may be used to achieve the desired long-term expression. Advantageously, the retroviral/lentiviral (e.g. SIV) vectors of the present invention can be used in gene therapy for CF, by providing a functional copy of the CFTR gene to ameliorate or prevent lung disease in CF patients, independent of the underlying mutation.

CFTR modulators are breakthrough therapies that target the underlying cause of CF, rather than ameliorating symptoms of the disease. However, current CFTR modulators are only effective in patients with specific mutations.

Therefore, combining the use of gene therapy with CFTR modulators offers a potentially significant advance in the treatment of CF. The present inventors are the first to investigate the effects of combining retroviral/lentiviral (e.g. SIV) vectors with CFTR modulators. In particular, the present inventors have shown that gene therapy with a retroviral/lentiviral (e.g. SIV) vectors of the present invention produces a greater than expected therapeutic effect when combined with a CFTR modulator. As exemplified herein, the inventors have surprisingly demonstrated that the effect of a CFTR modulator, particularly a CFTR potentiator, and rSIV.F/HN-CFTR combination is greater than the additive effects of the separate effects of the CFTR modulator, particularly the CFTR potentiator, and rSIV.F/HN-mediated CFTR expression.

Accordingly, retroviral/lentiviral (e.g. SIV) vectors with a CFTR transgene according to the invention may be used in combination with one or more CFTR modulator to treat CF.

Preferred embodiments relate to the therapeutic use of (A) a SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 16 (which comprises a CFTR transgene), preferably wherein the modified retroviral RNA sequence consists of a nucleic acid sequence of SEQ ID NO: 16; and (b) the F protein comprises a first subunit which comprises or consists of an amino acid sequence of SEQ ID NO: 19 and a second subunit which comprises or consists of an amino acid sequence of SEQ ID NO: 20 in combination with (B) a CFTR modulator selected from ivacaftor, tezacaftor, elexacaftor or lumacaftor, of a combination thereof.

Particularly preferred embodiments relate to the therapeutic use of (A) a SIV vector pseudotyped with Sendai virus hemagglutinin-neuraminidase (HN) and fusion (F) proteins, wherein: (a) said vector comprises a modified retroviral RNA sequence which comprises or consists of a nucleic acid sequence of SEQ ID NO: 16 (which comprises a CFTR transgene), preferably wherein the modified retroviral RNA sequence consists of a nucleic acid sequence of SEQ ID NO: 16; and (b) the F protein comprises a first subunit which comprises or consists of an amino acid sequence of SEQ ID NO: 19 and a second subunit which comprises or consists of an amino acid sequence of SEQ ID NO: 20 in combination with (B) the CFTR modulator (specifically the CFTR potentiator) ivacaftor.

A retroviral/lentiviral (e.g. SIV) vector and one or more CFTR modulator may be administered to a patient with CF who is exhibiting one or more symptom of CF. When administered to such a patient, a retroviral/lentiviral (e.g. SIV) vector and one or more CFTR modulator can cure, delay, reduce the severity of, or ameliorate one or more symptoms, and/or prolong the survival of a patient beyond that expected in the absence of such treatment and/or beyond that expected using a conventional CF treatment (e.g. a CFTR modulator, particularly a CFTR potentiator, alone). Thus, retroviral/lentiviral (e.g. SIV) vector and one or more CFTR modulator, particularly a CFTR potentiator, may be administered to a patient with CF to ameliorate the disease and/or prolong the survival of a patient with CF beyond that expected in the absence of such treatment and/or beyond that expected using a conventional CF treatment (e.g. a CFTR modulator or CFTR potentiator alone).

The retroviral/lentiviral (e.g. SIV) vector and one or more CFTR modulator are administered in combination. Administered “in combination,” encompasses both simultaneous (also referred to as concurrent) administration/delivery and sequential (also referred to as separate) administration/delivery.

For, “simultaneous” or “concurrent delivery”, the delivery of the retroviral/lentiviral (e.g. SIV) vector may still be occurring when the delivery of the CFTR modulator begins, or the delivery of CFTR modulator may still be occurring when the delivery of the retroviral/lentiviral (e.g. SIV) vector begins, so that there is overlap in terms of administration. Simultaneous delivery may encompass delivery of the retroviral/lentiviral (e.g. SIV) vector and CFTR modulator within weeks to months or even years of each other, typically so that the retroviral/lentiviral (e.g. SIV) vector delivery overlaps with the delivery of the CFTR modulator.

Alternatively, the delivery of the retroviral/lentiviral (e.g. SIV) vector may end before the delivery of the CFTR modulator begins, or the delivery of the CFTR modulator may end before delivery of the retroviral/lentiviral (e.g. SIV) vector begins. Sequential administration may involve the retroviral/lentiviral (e.g. SIV) vector and CFTR modulator being administered within 30 minutes, 1 hour, 2 hours, 3 hours, 4 hours, 6 hours, 12 hours or 24 hours, 1 week, 2 weeks, 1 month, 2 months, or longer of each other.

The CFTR modulator may be administered at an interval of once every hour, once every 2 hours, once every 3 hours, once every 4 hours, once every 6 hours, once every 8 hours, once every 12 hours, daily, once every 2 days or more. Typically the CFTR modulator is administered every 12 hours.

The retroviral/lentiviral (e.g. SIV) vector may be administered once every month, once every 2 months, once every 3 months, once every 4 months, once every 6 months, once every 8 months, once every 12 months or more. As the frequency of administration of the retroviral/lentiviral (e.g. SIV) vector is lower than the frequency of administration of the CFTR modulator, administration of the combination therapy is typically by sequential administration.

Treatment with a retroviral/lentiviral (e.g. SIV) vector and/or CFTR modulator at the desired dosing frequency may be continued for as long as required, for example, for at least six months, at least one year, two years, three years, four years, five years, ten years, fifteen years, twenty years, or more, up to for the lifetime of the patient to be treated.

Typically the treatment is more effective because of combined administration. For example, treatment with the CFTR modulator may be more effective, e.g., an equivalent effect is seen with less of the CFTR modulator, or the CFTR modulator reduces symptoms to a greater extent, than would be seen if the CFTR modulator were administered in the absence of the retroviral/lentiviral (e.g. SIV) vector, or the analogous situation is seen with the retroviral/lentiviral (e.g. SIV) vector. Typically, delivery is such that the reduction in a symptom, or other parameter related to CF is greater than what would be observed with the CFTR modulator delivered in the absence of the retroviral/lentiviral (e.g. SIV) vector, or the analogous situation is seen with the retroviral/lentiviral (e.g. SIV) vector.

It will be appreciated that appropriate dosage of the retroviral/lentiviral (e.g. SIV) vector may and/or the CFTR modulator, will depend on the specific agent, and can also vary from patient to patient.

It will be appreciated that appropriate dosage of the retroviral/lentiviral (e.g. SIV) vector and/or CFTR modulator, will depend on the specific agent, and can also vary from patient to patient.

Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects. Non-limiting exemplary dosages and routes of administration are described herein.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

The duration of action of a combination therapy according to the invention may be for at least 6 hours, at least 12 hours, at least 18 hours, at least 24 hours, at least 48 hours, at least 72 hours, at least 4 days, at least 5 days, at least 6 days, at least 1 week, at least 2 weeks, at least 3 weeks, at least 4 weeks, at least 6 weeks, at least 8 weeks, at least 12 weeks, at least six months, at least 1 year or more. Typically this is assessed relative to the last administration of the retroviral/lentiviral (e.g. SIV) vector and/or CFTR modulator, particularly the last administration of the retroviral/lentiviral (e.g. SIV) vector.

A retroviral/lentiviral (e.g. SIV) vector according to the invention is typically administered by inhalation. Accordingly, said retroviral/lentiviral (e.g. SIV) vector may be formulated for inhalation, as described herein. The one or more CFTR modulator may be administered by any appropriate route, and may be formulated accordingly. In particular, the one or more CFTR modulator may be administered orally, and may be formulated for oral administration.

Accordingly, the invention provides a method of treating CF in a subject in need thereof, said method comprising administering to the subject a therapeutically effective amount of each of (i) a retroviral/lentiviral (e.g. SIV) vector of the invention; and (ii) one or more CFTR modulator. Any retroviral/lentiviral (e.g. SIV) vector as described herein may be used in combination with any one or more CFTR modulator, such as those described and exemplified herein.

The combination therapies of the invention may restore CFTR expression and/or activity to a level which provides a therapeutic benefit. A combination therapy may restore (increase) CFTR expression and/or activity to a level which matches or exceeds CFTR expression and/or activity in a healthy control. However, restoration of healthy CFTR expression and/or activity is not essential to achieve a therapeutic benefit. Rather, the therapeutic threshold, i.e. the level above which a therapeutic benefit is achieved may be lower than the level of CFTR expression and/or activity in a healthy control. Indeed, patients, particularly those with a class I CFTR mutation resulting in null CFTR expression, may receive a therapeutic benefit from even relatively small increases in CFTR expression and/or activity (e.g. 5% or 10% CFTR expression and/or activity compared to healthy control expression levels).

A retroviral/lentiviral (e.g. SIV) vector as described herein, particularly in the context of a combination therapy of the invention may increase (restore) CFTR expression (particularly cellular CFTR expression levels and/or global expression in the lungs or respiratory tree), CFTR activity and/or CFTR current as described herein. Any combination of increase in CFTR expression (cellular and/or global), CFTR activity and/or CFTR current, including the quantified increases described below is encompassed by the present invention.

A retroviral/lentiviral (e.g. SIV) vector as described herein, particularly in the context of a combination therapy of the invention may increase (restore) CFTR expression (particularly cellular CFTR expression levels and/or global expression in the lungs or respiratory tree) to at least 5%, at least 10%, at least 20%, at least 30%, at least 40%, at least 50%, at least 60%, at least 70%, at least 75%, at least 80%, at least 90%, at least 95%, at least 100%, at least 120% or more of CFTR expression in a healthy control. Typically a retroviral/lentiviral (e.g. SIV) vector as described herein, particularly in the context of a combination therapy of the invention may increase (restore) CFTR expression, particularly CFTR cellular expression levels, to at least 20%, preferably at least 50%, more preferably at least 75% of CFTR expression in a healthy control. A retroviral/lentiviral (e.g. SIV) vector as described herein, particularly in the context of a combination therapy of the invention may increase (restore) CFTR expression, particularly global CFTR expression in the lungs, to at least 5%, at least 10%, preferably at least 20% of CFTR expression in a healthy control. Expression levels of a transgene and/or the encoded therapeutic protein of the invention may be measured in the lung tissue, A high and/or therapeutic expression level may therefore refer to the concentration in the lung. CFTR expression may be quantified using one or more of the following techniques: CFTR RNA expression in lung tissue, CFTR protein expression in lung tissue, PK assays (vector copy number, integration), DNA content in sputum (as surrogate for NETosis).

Other endpoints for efficacy assessment of treatment according to the invention include: improvement in FEV1 (lung function); MRI and/or CT for efficacy assessment; reduced expression of one or more biomarker of inflammation, such as IL-8, IL1beta, IL-6, TNFalpha (typically measured in sputum), calprotectin (typically measured in serum); differential cell count in sputum; reduced surfactant protein D (SP-D) in serum as marker for reduced epithelial injury; reduction of pulmonary exacerbations; improvement of Lung Clearance index, Quality of Life as patient reported outcome via CFQ-R respiratory domain (and/or other appropriate questionnaires as well), exploratory imaging endpoints (to show improved lung ventilation, mucus plugging and/or others, e.g. via Eichinger score of mRI).

Alternatively or in addition, a combination therapy of the invention may increase (restore) CFTR activity (particularly cellular CFTR activity and/or global activity in the lungs or respiratory tree) to at least 5%, at least 10%, at least 20%, at least 30%, at least 40%, at least 50%, at least 60%, at least 70%, at least 75%, at least 80%, at least 90%, at least 95%, at least 100%, at least 120% or more of CFTR activity in a healthy control. Typically a combination therapy of the invention may increase (restore) CFTR activity, particularly CFTR cellular activity, to at least 20%, preferably at least 50%, more preferably at least 75% of CFTR activity in a healthy control. A retroviral/lentiviral (e.g. SIV) vector as described herein, particularly in the context of a combination therapy of the invention may increase (restore) CFTR activity, particularly global CFTR expression in the lungs, to at least 5%, at least 10%, preferably at least 20% of CFTR activity in a healthy control.

As used herein, typically a healthy control is an equivalent individual or population who do not have CF. Preferably a healthy control is an equivalent individual or population who do not have CF and is otherwise in good health. A healthy control may be matched with the subject using standard clinical methodology (e.g. age/sex matching, or matching based on other criteria).

Other controls may be an equivalent individual with CF who has been treated with either the retroviral/lentiviral (e.g. SIV) vector or CFTR modulator alone.

CFTR activity may be defined in terms of the CFTR RNA expression in lung tissue; CFTR protein expression in lung tissue and/or activity of the CFTR channel itself (e.g. by electrophysiological measurements using patient's bronchial brushings).

A combination therapy of the invention may increase (restore) CFTR current by at least 1.2 fold, at least 1.3 fold, at least 1.4 fold, at least 1.5 fold, at least 2 fold or more compared with treatment with the retroviral/lentiviral (e.g. SIV) vector alone (i.e. compared with the increase in CFTR current achieved when treating with the retroviral/lentiviral (e.g. SIV) alone). A combination therapy of the invention may increase (restore) CFTR current by between about 1.3 fold to about 3 fold or between about 1.2 fold to about 2 fold compared with treatment with the retroviral/lentiviral (e.g. SIV) vector alone (i.e. compared with the increase in CFTR current achieved when treating with the retroviral/lentiviral (e.g. SIV) alone). Preferably, a combination therapy of the invention may increase (restore) CFTR current by at least about 1.2 fold, at least about 1.3 fold, at least about 1.5 fold, at least about 1.8 fold, such as by about 1.3 fold to about 1.8 fold compared with treatment with the retroviral/lentiviral (e.g. SIV) vector alone.

A combination therapy of the invention may increase (restore) CFTR current to at least 5%, at least 10%, at least 20%, at least 30%, at least 40%, at least 50%, at least 60%, at least 70%, at least 75%, at least 80%, at least 90%, at least 95%, at least 100%, at least 120% or more of CFTR current in a healthy control. Typically a combination therapy of the invention may increase (restore) CFTR cellular current to at least 20%, preferably at least 50%, more preferably at least 75% of CFTR current in a healthy control. A retroviral/lentiviral (e.g. SIV) vector as described herein, particularly in the context of a combination therapy of the invention may increase (restore) CFTR current, particularly globally in the lungs, to at least 5%, at least 10%, preferably at least 20% of CFTR current in a healthy control.

The invention provides a combination of a retroviral/lentiviral (e.g. SIV) and one or more CFTR modulator as described herein for use in treating CF, wherein the treatment: (i) restores cellular CFTR activity to at least 10% or at least 50% of the CFTR activity (e.g. at least 70%) in a healthy control; (ii) restores global CFTR activity in the lungs to at least 5% or at least 10% of the CFTR activity (e.g. at least 20%) in a healthy control; and/or (iii) increases CFTR current by at least about 1.3 fold (e.g. from about 1.3 fold to about 1.8 fold, from about 1.3 fold to about 3 fold, or about 1.3 fold) compared with treatment with the retroviral/lentiviral (e.g. SIV) vector alone. Typically the invention provides a combination of a retroviral/lentiviral (e.g. SIV) and one or more CFTR modulator as described herein for use in treating CF, wherein the treatment: (i) restores global CFTR activity in the lungs to at least 5% or at least 10% of the CFTR activity (e.g. at least 20%) in a healthy control; and/or (ii) increases CFTR current by at least about 1.3 fold (e.g. from about 1.3 fold to about 1.8 fold, from about 1.3 fold to about 3 fold, or about 1.3 fold) compared with treatment with the retroviral/lentiviral (e.g. SIV) vector alone.

The invention provides a combination of a retroviral/lentiviral (e.g. SIV) and one or more CFTR modulator as described herein for use in treating CF, wherein the patient to be treated has at least one class I CFTR mutation and the treatment: (i) restores cellular CFTR activity to at least 10% or at least 50% of the CFTR activity (e.g. at least 70%) in a healthy control; (ii) restores global CFTR activity in the lungs to at least 5% or at least 10% of the CFTR activity (e.g. at least 20%) in a healthy control; and/or (iii) increases CFTR current by at least about 1.3 fold (e.g. from about 1.3 fold to about 1.8 fold, from about 1.3 fold to about 3 fold, or about 1.3 fold) compared with treatment with the retroviral/lentiviral (e.g. SIV) vector alone. Typically the invention provides a combination of a retroviral/lentiviral (e.g. SIV) and one or more CFTR modulator as described herein for use in treating CF, wherein the patient to be treated has at least one class I CFTR mutation and the treatment: (i) restores global CFTR activity in the lungs to at least 5% or at least 10% of the CFTR activity (e.g. at least 20%) in a healthy control; and/or (ii) increases CFTR current by at least about 1.3 fold (e.g. from about 1.3 fold to about 1.8 fold, from about 1.3 fold to about 3 fold, or about 1.3 fold) compared with treatment with the retroviral/lentiviral (e.g. SIV) vector alone.

The invention provides a combination of a retroviral/lentiviral (e.g. SIV) and one or more CFTR modulator as described herein for use in treating CF, wherein the patient to be treated has at least one class II CFTR mutation and the treatment: (i) restores cellular CFTR activity to at least 10% or at least 50% of the CFTR activity (e.g. at least 70%) in a healthy control; (ii) restores global CFTR activity in the lungs to at least 5% or at least 10% of the CFTR activity (e.g. at least 20%) in a healthy control; and/or (ii) increases CFTR current by at least about 1.3 fold (e.g. from about 1.3 fold to about 1.8 fold, from about 1.3 fold to about 3 fold, or about 1.3 fold) compared with treatment with the retroviral/lentiviral (e.g. SIV) vector alone. Typically the invention provides a combination of a retroviral/lentiviral (e.g. SIV) and one or more CFTR modulator as described herein for use in treating CF, wherein the patient to be treated has at least one class II CFTR mutation and the treatment: (i) restores global CFTR activity in the lungs to at least 5% or at least 10% of the CFTR activity (e.g. at least 20%) in a healthy control; and/or (ii) increases CFTR current by at least about 1.3 fold (e.g. from about 1.3 fold to about 1.8 fold, from about 1.3 fold to about 3 fold, or about 1.3 fold) compared with treatment with the retroviral/lentiviral (e.g. SIV) vector alone.

A retroviral/lentiviral (e.g. SIV) described herein, typically as part of a combination therapy of the invention, may transduce of airway epithelial cells with an transduction rate sufficient to achieve a therapeutic effect on CFTR expression and/or activity. Typically, a retroviral/lentiviral (e.g. SIV) described herein, typically as part of a combination therapy of the invention, may transduce airway epithelial cells at a transduction rate of at least about 5%, at least about 7%, at least about 10%, at least about 15%, at least about 20% or more, such as from about 10% to about 20% (i.e. about 10%, 11%, 12%, 13%, 14%, 15%, 16%, 17%, 18%, 19% or 20%). Preferably a retroviral/lentiviral (e.g. SIV) described herein, typically as part of a combination therapy of the invention, may transduce of airway epithelial cells at a transduction rate of from about 1% to about 50% (i.e. about 14%, 15%, 17%, 18%, 20%, 25%, 30%, 35%, 40% or 45%). As defined herein, the term “airway epithelial cell” encompasses any cell found within the airway epithelium, as described herein, including but not limited to basal cells and submucosal gland duct cells in the upper airways, goblet, club cells and neuroendocrine cells in the bronchiolar airways, bronchioalveolar stem cells in the terminal bronchioles and type II pneumocytes in the alveoli, and any combination thereof.

A retroviral/lentiviral (e.g. SIV) vector as described herein, particularly in the context of a combination therapy of the invention may achieve a VCN of at least 1 copy/cell, 2 copies/cell, 3 copies/cell, 4 copies/cell, 5 copies/cell, 6 copies/cell, 7 copies/cell, 8, copies per cell, 9 copies per cell, at least 10 copies/cell or more.

In some embodiments, the invention relates to the use of retroviral/lentiviral (e.g. SIV) vectors with a CFTR transgene according to the invention used in combination with one or more CFTR modulator, wherein said combination does not further comprise a LasB inhibitor. In other words, the invention relates to the treatment of CF using a retroviral/lentiviral (e.g. SIV) vector with a CFTR transgene according to the invention used in combination with one or more CFTR modulator, with the proviso that a LasB inhibitor is not used in said method. Particularly, in such embodiments the use of indanes as a LasB inhibitor is disclaimed, such as those disclosed in WO 2021/191240.

The invention also provides a combination of a retroviral/lentiviral (e.g. SIV) vector as described herein and one or more CFTR modulator for use in a method of treating CF. Any retroviral/lentiviral (e.g. SIV) vector as described herein may be used in combination with any one or more CFTR modulator, such as those described and exemplified herein.

The invention also provides the use of a retroviral/lentiviral (e.g. SIV) vector as described herein in the manufacture of a medicament for use in a method of treating CF, wherein said method of treatment further comprises the administration of one or more CFTR modulator. The invention also provides the use of a CFTR modulator as described herein in the manufacture of a medicament for use in a method of treating CF, wherein said method of treatment further comprises the administration of a retroviral/lentiviral (e.g. SIV) vector. Any retroviral/lentiviral (e.g. SIV) vector as described herein may be used in combination with any one or more CFTR modulator, such as those described and exemplified herein.

### Formulation and Administration

The retroviral/lentiviral (e.g. SIV) vectors and the CFTR modulators of the invention may each independently be administered in any dosage appropriate for achieving the desired therapeutic effect. Appropriate dosages may be determined by a clinician or other medical practitioner using standard techniques and within the normal course of their work.

Non-limiting examples of suitable dosages of the retroviral/lentiviral (e.g. SIV) vectors include from about 1×10⁶ (which may also be written as 10⁶) transducing units (TU) to about 1×10¹⁴ (which may also be written as 10¹⁴) TU, preferably from between about 10⁶ TU to about 10¹² TU, such as about 10⁶ TU, 1.5×10⁶ TU, 10⁷ TU, 1.5×10⁷ TU, 10⁸ TU, 1.5×10⁸ TU, 5×10⁸ TU, 8×10⁸ TU, 10⁹ TU, 1.5×10⁹ TU, 10¹⁰ TU, 1.5×10¹⁰ TU, 10¹¹ TU, 1.5×10¹¹ TU or more. Preferred dose ranges include between about 8⁸ to about 10¹⁴ TU, or between about 10⁶ to about 10¹² TU, These doses may be administered at any dosing interval determined by the treating clinician, such as those dosing intervals described herein (e.g. at a frequency of every 3 months, every 6 months, every 12 months, every 24 months, every 36 months or every 48 months). By way of non-limiting example, a dose of about 10⁶ TU may be administered once every 6 months. By way of a further non-limiting example, a dose of about 10¹⁰ TU may be administered every 12 months.

Each CFTR modulator may be administered at the standard dose indicated for single therapy of CF with said CFTR modulator, i.e. at an approved or standard dose/concentration for monotherapy with said modulator. Each CFTR modulator may be administered as part of combination therapy according to the invention at a dose lower than the standard dose indicated for single therapy with said CFTR modulator, i.e. at concentration lower than an approved or standard dose/concentration for monotherapy with said modulator. A CFTR modulator may be administered at a dose of between about 5 mg to about 200 mg, such as between about 5 mg to about 150 mg, between about 25 mg to about 150 mg, or between about 75 mg to about 150 mg. These doses may be administered at any dosing interval determined by the treating clinician, such as those dosing intervals described herein (e.g. at a frequency of every 4 hours, every 8 hours, or every 12 hours, preferably every 12 hours).

By way of non-limiting example, ivacaftor may be administered at a dose of from about 5 mg to about 150 mg, preferably from about 25 mg to about 150 mg, such as about 150 mg every 12 hours. By way of a further non-limiting example, for paediatric dosing ivacaftor may be administered at a dose of about 75 mg every 12 hours.

By way of a further non-limiting example, Trikafta® (elexacaftor+tezacaftor+ivacaftor) may be administered every 12 hours, with a first (typically morning) dose of Trikafta® typically comprising about 200 mg elexacaftor, about 100 mg tezacaftor and about 150 mg ivacaftor (e.g. in the form of 2 tablets each containing elexacaftor+tezacaftor+ivacaftor), and a second (typically evening) dose of about 150 mg ivacaftor (e.g. in the form of 2 tablets).

By way of a further non-limiting example, Orkambi® (lumacaftor+ivacaftor) may be administered every 12 hours, with each dose of Orkambi® typically comprising about 400 mg lumacaftor and about 250 mg ivacaftor (e.g. in the form of 2 tablets each containing lumacaftor+ivacaftor). By way of a further non-limiting example, for paediatric dosing Orkambi® may be administered every 12 hours, with each dose of Orkambi® typically comprising about 200 mg lumacaftor and about 250 mg ivacaftor (e.g. in the form of 2 tablets each containing lumacaftor+ivacaftor).

By way of a further non-limiting example, Symdeko® (tezacaftor+ivacaftor) may be administered every 12 hours, with a first (typically morning) dose of Symdeko® typically comprising about 100 mg tezacaftor and about 150 mg ivacaftor (e.g. in the form of a single tablet containing tezacaftor+ivacaftor), and a second (typically evening) dose of about 150 mg ivacaftor (e.g. in the form of 1 tablet).

Combination therapy according to the invention may use compositions comprising the retroviral/lentiviral (e.g. SIV) vectors described above, and a pharmaceutically-acceptable carrier. Typically said compositions are formulated for administration by inhalation.

The CFTR modulators used in the combination therapies of the invention may be appropriately formulated in a composition comprising a pharmaceutically-acceptable carrier. The CFTR modulators are typically formulated for administration orally.

Administration of CFTR modulators is generally by conventional routes e.g. oral, intravenous, subcutaneous, intraperitoneal, or mucosal routes. The administration may be by parenteral injection, for example, a subcutaneous, intradermal or intramuscular injection. For example, CFTR modulators may be particularly suited to administration orally. Administration of small molecule CFTR modulators may be injection, such as intravenously, intramuscularly, intradermally, or subcutaneously, or preferably by oral administration (small molecules with molecule weight of less than 500 Da typically exhibiting oral bioavailability).

CFTR modulators may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

Preferably CFTR modulators are prepared as for oral administration. A CFTR modulator may be encapsulated within an oral dosage form. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

The active ingredients (such as the CFTR modulators used in the combination therapies of the invention) are often mixed with excipients which are pharmaceutically acceptable and compatible with the active ingredient. Suitable excipients are, for example, water, saline, dextrose, glycerol, ethanol, or the like and combinations thereof. In addition, if desired, the composition may contain minor amounts of auxiliary substances such as wetting or emulsifying agents, pH buffering agents, and/or adjuvants which enhance the effectiveness of the CFTR modulators.

Non-limiting examples of pharmaceutically acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long-term storage.

Preferably the CFTR modulators used in combination therapies of the invention are formulated for oral administration and are administered orally. Thus, the invention typically relates to combination therapies using two separate formulations, one comprising the retroviral/lentiviral (e.g. SIV) vectors (typically for inhalative administration) and one comprising the one or more CFTR modulator (typically for oral administration).

The retroviral/lentiviral (e.g. SIV) vectors of the invention and/or the one or more CFTR modulator may each independently be administered by any appropriate route. It may be desired to direct the compositions of the present invention (as described above) to the respiratory system of a subject. Efficient transmission of a therapeutic/prophylactic composition or medicament to the site of infection in the respiratory tract may be achieved by oral administration or by inhalation, for example, as aerosols, or by catheters. Typically the retroviral/lentiviral (e.g. SIV) vectors of the invention are administered by inhalation, and as such are preferably stable in clinically relevant nebulisers, inhalers (including metered dose inhalers), catheters and aerosols, etc. Typically the one or more CFTR modulator is administered orally.

Formulations for inhalative administration may be in the form of droplets, and may be administered by nebulisation using a suitable device. A formulation for inhalative administration may comprise droplets having approximate mass median aerodynamic diameters (MMADs) in the range of 0.1-50 μm, such as 1-25 μm, 1-10 μm, or 1-5 μm, particularly 1-10 μm. Alternatively, in terms of volume, the droplets may be in the range of about 0.001-100 μl, such as 0.1-50 μl or 1.0-25 μl, or such as 0.001-1 μl.

The aerosol formulation may take the form of a powder, suspension or solution. The size of aerosol droplets is relevant to the delivery capability of an aerosol. Smaller droplets may travel further down the respiratory airway towards the alveoli than would larger particles. In one embodiment, the aerosol droplets have a diameter distribution to facilitate delivery along the entire length of the trachea, bronchi and bronchioles i.e. the conducting airways. Alternatively, the droplet size distribution may be selected to target a particular section of the respiratory airway, for example the bronchioles or alveoli. In the case of aerosol delivery of the medicament, the droplets may have diameters in the approximate range of 0.1-50 μm, preferably 1-25 μm, more preferably 1-10 μm or 1-5 μm.

Aerosol particles may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J. in Remington's Pharmaceutical Sciences (supra). The agents may be formulated as solution aerosols, dispersion or suspension aerosols of dry powders, emulsions or semisolid preparations. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising a vector of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

As described herein, in some cases after an initial administration a subsequent administration of a retroviral/lentiviral (e.g. SIV) vector may be performed. The administration may, for instance, be at least a week, two weeks, a month, two months, three months, four months, six months, a year or more after the initial administration. In some instances, retroviral/lentiviral (e.g. SIV) vector of the invention may be administered at least once a week, once a fortnight, once a month, every two months, every six months, annually or at longer intervals. Preferably, administration is every six months, more preferably annually. The retroviral/lentiviral (e.g. SIV) vectors may, for instance, be administered at intervals dictated by when the effects of the previous administration are decreasing. Administration of the retroviral/lentiviral (e.g. SIV) vectors at the desired frequency may continue for the life of the patient.

Also as described herein, the CFTR modulator is typically administered on an on-going basis at a frequency as described herein. The CFTR modulator may, for instance, be administered at intervals dictated by when the effects of the previous administration are decreasing. Administration of the CFTR modulator at the desired frequency may continue for the life of the patient.

The combination therapy of the present invention may be combined with one or more additional treatment for CF, including one or more additional CF modulator, bronchodilators, steroids, agents which thin or clear mucus or other pulmonary secretions, antibiotics and/or airway clearance techniques such as active cycle of breathing techniques (ACBT) and autogenic drainage. The one or more additional treatment for CF may be administered sequentially or simultaneously (as defined herein) with the combination therapy of the invention.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Wa Ile et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}\begin{matrix}[ length of the longer sequence plus the number of gaps \\ introduced into the longer sequence in order to align the two\end{matrix} \\ sequences ]\end{matrix}} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labelling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### Sequence Information

### Key to Sequences

SEQ ID NO: 1 Exemplified CFTR transgene (soCFTR2)
SEQ ID NO: 2 Exemplified hCEF promoter
SEQ ID NO: 3 Exemplified CMV promoter
SEQ ID NO: 4 Exemplified EF1a promoter
SEQ ID NO: 5 wild-type SIV gag-pol nucleic acid sequence
SEQ ID NO: 6 codon-optimised SIV gal-pol nucleic acid sequence
SEQ ID NO: 7 Plasmid as defined in FIG. 2C (pDNA2a pGM691)
SEQ ID NO: 8 Plasmid as defined in FIG. 2A (pDNA1 pGM326)
SEQ ID NO: 9 Plasmid as defined in FIG. 2B (pDNA1 pGM830)
SEQ ID NO: 10 Plasmid as defined in FIG. 2G (pDNA2a pGM297)
SEQ ID NO: 11 Plasmid as defined in FIG. 2D (pDNA2b pGM299)
SEQ ID NO: 12 Plasmid as defined in FIG. 2E (pDNA3a pGM301)
SEQ ID NO: 13 Plasmid as defined in FIG. 2F (pDNA3b pGM303)
SEQ ID NO: 14 Exemplified WPRE component (mWPRE)
SEQ ID NO: 15 Exemplary CAG promoter
SEQ ID NO: 16 modified SIV/CFTR RNA sequence
SEQ ID NO: 17 Fct4 protein
SEQ ID NO: 18 Fct4 protein (including signal sequence)
SEQ ID NO: 19 Fct4 protein (fragment 1)
SEQ ID NO: 20 Fct4 protein (fragment 2)
SEQ ID NO: 21 Fct4 protein signal sequence
SEQ ID NO: 22 p17 protein sequence
SEQ ID NO: 23 p24 protein sequence
SEQ ID NO: 24 p8 protein sequence
SEQ ID NO: 25 Protease sequence
SEQ ID NO: 26 p51 protein sequence
SEQ ID NO: 27 p15 protein sequence
SEQ ID NO: 28 p31 protein sequence
SEQ ID NO: 29 Gag protein
SEQ ID NO: 30 Pol protein

### Sequences

SEQ ID NO: 1 Exemplified CFTR transgene (soCFTR2)

| | |
|---|---|
| 1 | GCTAGCCACC ATGCAGAGAA GCCCTCTGGA GAAGGCCTCT GTGGTGAGCA AGCTGTTCTT |
| 61 | CAGCTGGACC AGGCCCATCC TGAGGAAGGG CTACAGGCAG AGACTGGAGC TGTCTGACAT |
| 121 | CTACCAGATC CCCTCTGTGG ACTCTGCTGA CAACCTGTCT GAGAAGCTGG AGAGGGAGTG |
| 181 | GGATAGAGAG CTGGCCAGCA AGAAGAACCC CAAGCTGATC AATGCCCTGA GGAGATGCTT |
| 241 | CTTCTGGAGA TTCATGTTCT ATGGCATCTT CCTGTACCTG GGGGAAGTGA CCAAGGCTGT |
| 301 | GCAGCCTCTG CTGCTGGGCA GAATCATTGC CAGCTATGAC CCTGACAACA AGGAGGAGAG |
| 361 | GAGCATTGCC ATCTACCTGG GCATTGGCCT GTGCCTGCTG TTCATTGTGA GGACCCTGCT |
| 421 | GCTGCACCCT GCCATCTTTG GCCTGCACCA CATTGGCATG CAGATGAGGA TTGCCATGTT |
| 481 | CAGCCTGATC TACAAGAAAA CCCTGAAGCT GTCCAGCAGA GTGCTGGACA AGATCAGCAT |
| 541 | TGGCCAGCTG GTGAGCCTGC TGAGCAACAA CCTGAACAAG TTTGATGAGG GCCTGGCCCT |
| 601 | GGCCCACTTT GTGTGGATTG CCCCTCTGCA GGTGGCCCTG CTGATGGGCC TGATTTGGGA |
| 661 | GCTGCTGCAG GCCTCTGCCT TTTGTGGCCT GGGCTTCCTG ATTGTGCTGG CCCTGTTTCA |
| 721 | GGCTGGCCTG GGCAGGATGA TGATGAAGTA CAGGGACCAG AGGGCAGGCA AGATCAGTGA |
| 781 | GAGGCTGGTG ATCACCTCTG AGATGATTGA GAACATCCAG TCTGTGAAGG CCTACTGTTG |
| 841 | GGAGGAAGCT ATGGAGAAGA TGATTGAAAA CCTGAGGCAG ACAGAGCTGA AGCTGACCAG |
| 901 | GAAGGCTGCC TATGTGAGAT ACTTCAACAG CTCTGCCTTC TTCTTCTCTG GCTTCTTTGT |
| 961 | GGTGTTCCTG TCTGTGCTGC CCTATGCCCT GATCAAGGGG ATCATCCTGA GAAAGATTTT |
| 1021 | CACCACCATC AGCTTCTGCA TTGTGCTGAG GATGGCTGTG ACCAGACAGT TCCCCTGGGC |
| 1081 | TGTGCAGACC TGGTATGACA GCCTGGGGGC CATCAACAAG ATCCAGGACT TCCTGCAGAA |
| 1141 | GCAGGAGTAC AAGACCCTGG AGTACAACCT GACCACCACA GAAGTGGTGA TGGAGAATGT |
| 1201 | GACAGCCTTC TGGGAGGAGG GCTTTGGGGA GCTGTTTGAG AAGGCCAAGC AGAACAACAA |
| 1261 | CAACAGAAAG ACCAGCAATG GGGATGACTC CCTGTTCTTC TCCAACTTCT CCCTGCTGGG |
| 1321 | CACACCTGTG CTGAAGGACA TCAACTTCAA GATTGAGAGG GGGCAGCTGC TGGCTGTGGC |
| 1381 | TGGATCTACA GGGGCTGGCA AGACCAGCCT GCTGATGATG ATCATGGGGG AGCTGGAGCC |
| 1441 | TTCTGAGGGC AAGATCAAGC ACTCTGGCAG GATCAGCTTT TGCAGCCAGT TCAGCTGGAT |
| 1501 | CATGCCTGGC ACCATCAAGG AGAACATCAT CTTTGGAGTG AGCTATGATG AGTACAGATA |
| 1561 | CAGGAGTGTG ATCAAGGCCT GCCAGCTGGA GGAGGACATC AGCAAGTTTG CTGAGAAGGA |
| 1621 | CAACATTGTG CTGGGGGAGG GAGGCATTAC ACTGTCTGGG GGCCAGAGAG CCAGAATCAG |
| 1681 | CCTGGCCAGG GCTGTGTACA AGGATGCTGA CCTGTACCTG CTGGACTCCC CCTTTGGCTA |
| 1741 | CCTGGATGTG CTGACAGAGA AGGAGATTTT TGAGAGCTGT GTGTGCAAGC TGATGGCCAA |
| 1801 | CAAGACCAGA ATCCTGGTGA CCAGCAAGAT GGAGCACCTG AAGAAGGCTG ACAAGATCCT |
| 1861 | GATCCTGCAT GAGGGCAGCA GCTACTTCTA TGGGACCTTC TCTGAGCTGC AGAACCTGCA |
| 1921 | GCCTGACTTC AGCTCTAAGC TGATGGGCTG TGACAGCTTT GACCAGTTCT CTGCTGAGAG |
| 1981 | GAGGAACAGC ATCCTGACAG AGACCCTGCA CAGATTCAGC CTGGAGGGAG ATGCCCCTGT |
| 2041 | GAGCTGGACA GAGACCAAGA AGCAGAGCTT CAAGCAGACA GGGGAGTTTG GGGAGAAGAG |
| 2101 | GAAGAACTCC ATCCTGAACC CCATCAACAG CATCAGGAAG TTCAGCATTG TGCAGAAAAC |
| 2161 | CCCCCTGCAG ATGAATGGCA TTGAGGAAGA TTCTGATGAG CCCCTGGAGA GGAGACTGAG |
| 2221 | CCTGGTGCCT GATTCTGAGC AGGGAGAGGC CATCCTGCCT AGGATCTCTG TGATCAGCAC |
| 2281 | AGGCCCTACA CTGCAGGCCA GAAGGAGGCA GTCTGTGCTG AACCTGATGA CCCACTCTGT |
| 2341 | GAACCAGGGC CAGAACATCC ACAGGAAAAC CACAGCCTCC ACCAGGAAAG TGAGCCTGGC |
| 2401 | CCCTCAGGCC AATCTGACAG AGCTGGACAT CTACAGCAGG AGGCTGTCTC AGGAGACAGG |
| 2461 | CCTGGAGATT TCTGAGGAGA TCAATGAGGA GGACCTGAAA GAGTGCTTCT TTGATGACAT |
| 2521 | GGAGAGCATC CCTGCTGTGA CCACCTGGAA CACCTACCTG AGATACATCA CAGTGCACAA |
| 2581 | GAGCCTGATC TTTGTGCTGA TCTGGTGCCT GGTGATCTTC CTGGCTGAAG TGGCTGCCTC |
| 2641 | TCTGGTGGTG CTGTGGCTGC TGGGAAACAC CCCACTGCAG GACAAGGGCA ACAGCACCCA |
| 2701 | CAGCAGGAAC AACAGCTATG CTGTGATCAT CACCTCCACC TCCAGCTACT ATGTGTTCTA |
| 2761 | CATCTATGTG GGAGTGGCTG ATACCCTGCT GGCTATGGGC TTCTTTAGAG GCCTGCCCCT |
| 2821 | GGTGCACACA CTGATCACAG TGAGCAAGAT CCTCCACCAC AAGATGCTGC ACTCTGTGCT |
| 2881 | GCAGGCTCCT ATGAGCACCC TGAATACCCT GAAGGCTGGG GGCATCCTGA ACAGATTCTC |
| 2941 | CAAGGATATT GCCATCCTGG ATGACCTGCT GCCTCTCACC ATCTTTGACT TCATCCAGCT |
| 3001 | GCTGCTGATT GTGATTGGGG CCATTGCTGT GGTGGCAGTG CTGCAGCCCT ACATCTTTGT |
| 3061 | GGCCACAGTG CCTGTGATTG TGGCCTTCAT CATGCTGAGG GCCTACTTTC TGCAGACCTC |
| 3121 | CCAGCAGCTG AAGCAGCTGG AGTCTGAGGG CAGAAGCCCC ATCTTCACCC ACCTGGTGAC |
| 3181 | AAGCCTGAAG GGCCTGTGGA CCCTGAGAGC CTTTGGCAGG CAGCCCTACT TTGAGACCCT |
| 3241 | GTTCCACAAG GCCCTGAACC TGCACACAGC CAACTGGTTC CTCTACCTGT CCACCCTGAG |
| 3301 | ATGGTTCCAG ATGAGAATTG AGATGATCTT TGTCATCTTC TTCATTGCTG TGACCTTCAT |
| 3361 | CAGCATTCTG ACCACAGGAG AGGGAGAGGG CAGAGTGGGC ATTATCCTGA CCCTGGCCAT |
| 3421 | GAACATCATG AGCACACTGC AGTGGGCAGT GAACAGCAGC ATTGATGTGG ACAGCCTGAT |
| 3481 | GAGGAGTGTG AGCAGAGTGT TCAAGTTCAT TGATATGCCC ACAGAGGGCA AGCCTACCAA |
| 3541 | GAGCACCAAG CCCTACAAGA ATGGCCAGCT GAGCAAAGTG ATGATCATTG AGAACAGCCA |
| 3601 | TGTGAAGAAG GATGATATCT GGCCCAGTGG AGGCCAGATG ACAGTGAAGG ACCTGACAGC |
| 3661 | CAAGTACACA GAGGGGGGCA ATGCTATCCT GGAGAACATC TCCTTCAGCA TCTCCCCTGG |
| 3721 | CCAGAGAGTG GGACTGCTGG GAAGAACAGG CTCTGGCAAG TCTACCCTGC TGTCTGCCTT |
| 3781 | CCTGAGGCTG CTGAACACAG AGGGAGAGAT CCAGATTGAT GGAGTGTCCT GGGACAGCAT |
| 3841 | CACACTGCAG CAGTGGAGGA AGGCCTTTGG TGTGATCCCC CAGAAAGTGT TCATCTTCAG |
| 3901 | TGGCACCTTC AGGAAGAACC TGGACCCCTA TGAGCAGTGG TCTGACCAGG AGATTTGGAA |
| 3961 | AGTGGCTGAT GAAGTGGGCC TGAGAAGTGT GATTGAGCAG TTCCCTGGCA AGCTGGACTT |
| 4021 | TGTCCTGGTG GATGGGGGCT GTGTGCTGAG CCATGGCCAC AAGCAGCTGA TGTGCCTGGC |
| 4081 | CAGATCAGTG CTGAGCAAGG CCAAGATCCT GCTGCTGGAT GAGCCTTCTG CCCACCTGGA |
| 4141 | TCCTGTGACC TACCAGATCA TCAGGAGGAC CCTCAAGCAG GCCTTTGCTG ACTGCACAGT |
| 4201 | CATCCTGTGT GAGCACAGGA TTGAGGCCAT GCTGGAGTGC CAGCAGTTCC TGGTGATTGA |
| 4261 | GGAGAACAAA GTGAGGCAGT ATGACAGCAT CCAGAAGCTG CTGAATGAGA GGAGCCTGTT |
| 4321 | CAGGCAGGCC ATCAGCCCCT CTGATAGAGT GAAGCTGTTC CCCCACAGGA ACAGCTCCAA |
| 4381 | GTGCAAGAGC AAGCCCCAGA TTGCTGCCCT GAAGGAGGAG ACAGAGGAGG AAGTGCAGGA |
| 4441 | CACCAGGCTG TGAGGGCCC |

SEQ ID NO: 2 Exemplified hCEF promoter

| | |
|---|---|
| 1 | AGATCTGTTA CATAACTTAT GGTAAATGGC CTGCCTGGCT GACTGCCCAA TGACCCCTGC |
| 61 | CCAATGATGT CAATAATGAT GTATGTTCCC ATGTAATGCC AATAGGGACT TTCCATTGAT |
| 121 | GTCAATGGGT GGAGTATTTA TGGTAACTGC CCACTTGGCA GTACATCAAG TGTATCATAT |
| 181 | GCCAAGTATG CCCCCTATTG ATGTCAATGA TGGTAAATGG CCTGCCTGGC ATTATGCCCA |
| 241 | GTACATGACC TTATGGGACT TTCCTACTTG GCAGTACATC TATGTATTAG TCATTGCTAT |
| 301 | TACCATGGGA ATTCACTAGT GGAGAAGAGC ATGCTTGAGG GCTGAGTGCC CCTCAGTGGG |
| 361 | CAGAGAGCAC ATGGCCCACA GTCCCTGAGA AGTTGGGGGG AGGGGTGGGC AATTGAACTG |
| 421 | GTGCCTAGAG AAGGTGGGGC TTGGGTAAAC TGGGAAAGTG ATGTGGTGTA CTGGCTCCAC |
| 481 | CTTTTTCCCC AGGGTGGGGG AGAACCATAT ATAAGTGCAG TAGTCTCTGT GAACATTCAA |
| 541 | GCTTCTGCCT TCTCCCTCCT GTGAGTTTGC TAGC |

SEQ ID NO: 3 Exemplified CMV promoter
CCGCGGAGATCTCAATATTGGCCATTAGCCATATTATTCATTGGTTATATAGCATAAATCAATATTGGCT
ATTGGCCATTGCATACGTTGTATCTATATCATAATATGTACATTTATATTGGCTCATGTCCAATATGACC
GCCATGTTGGCATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAAGTCCGCCCCCTATT
GACGTCAATGACGGTAAATGGCCCGCCTGGCATTATGCCCAGTACATGACCTTACGGGACTTTCCTACTT
GGCAGTACATCTACGTATTAGTCATCGCTATTACCATGGTGATGCGGTTTTGGCAGTACACCAATGGGCG
TGGATAGCGGTTTGACTCACGGGGATTTCCAAGTCTCCACCCCATTGACGTCAATGGGAGTTTGTTTTGG
CACCAAAATCAACGGGACTTTCCAAAATGTCGTAATAACCCCGCCCCGTTGACGCAAATGGGCGGTAGGC
GTGTACGGTGGGAGGTCTATATAAGCAGAGCTCGTTTAGTGAACCGTCAGATCACTAGAAGCTTTATTGC
GGTAGTTTATCACAGTTAAATTGCTAACGCAGTCAGTGCTTCTGACACAACAGTCTCGAACTTAAGCTGC
AGAAGTTGGTCGTGAGGCACTGGGCAGGCTAGC
SEQ ID NO: 4 Exemplified EF1a promoter
AGATCCATATCCGCGGCAATTTTAAAAGAAAGGGAGGAATAGGGGGACAGACTTCAGCAGAGAGACTAATTAATA
TAATAACAACACAATTAGAAATACAACATTTACAAACCAAAATTCAAAAAATTTTAAATTTTAGAGCCGCGGAGA
TCCCGTGAGGCTCCGGTGCCCGTCAGTGGGCAGAGCGCACATCGCCCACAGTCCCCGAGAAGTTGGGGGGAGGGG
TCGGCAATTGAACCGGTGCCTAGAGAAGGTGGCGCGGGGTAAACTGGGAAAGTGATGTCGTGTACTGGCTCCGCC
TTTTTCCCGAGGGTGGGGGAGAACCGTATATAAGTGCAGTAGTCGCCGTGAACGTTCTTTTTCGCAACGGGTTTG
CCGCCAGAACACAGGCTAGC
SEQ ID NO: 5 wild-type SIV gag-pol nucleic acid sequence (from pGM297)
ATGGGGGCGGCTACCTCAGCACTAAATAGGAGACAATTAGACCAATTTGAGAAAATACGACTTCGCCCGAACGGA
AAGAAAAAGTACCAAATTAAACATTTAATATGGGCAGGCAAGGAGATGGAGCGCTTCGGCCTCCATGAGAGGTTG
TTGGAGACAGAGGAGGGGTGTAAAAGAATCATAGAAGTCCTCTACCCCCTAGAACCAACAGGATCGGAGGGCTTA
AAAAGTCTGTTCAATCTTGTGTGCGTACTATATTGCTTGCACAAGGAACAGAAAGTGAAAGACACAGAGGAAGCA
GTAGCAACAGTAAGACAACACTGCCATCTAGTGGAAAAAGAAAAAAGTGCAACAGAGACATCTAGTGGACAAAAG
AAAAATGACAAGGGAATAGCAGCGCCACCTGGTGGCAGTCAGAATTTTCCAGCGCAACAACAAGGAAATGCCTGG
GTACATGTACCCTTGTCACCGCGCACCTTAAATGCGTGGGTAAAAGCAGTAGAGGAGAAAAAATTTGGAGCAGAA
ATAGTACCCATGTTTCAAGCCCTATCAGAAGGCTGCACACCCTATGACATTAATCAGATGCTTAATGTGCTAGGA
GATCATCAAGGGGCATTACAAATAGTGAAAGAGATCATTAATGAAGAAGCAGCCCAGTGGGATGTAACACACCCA
CTACCCGCAGGACCCCTACCAGCAGGACAGCTCAGGGACCCTCGCGGCTCAGATATAGCAGGGACCACCAGCTCA
GTACAAGAACAGTTAGAATGGATCTATACTGCTAACCCCCGGGTAGATGTAGGTGCCATCTACCGGAGATGGATT
ATTCTAGGACTTCAAAAGTGTGTCAAAATGTACAACCCAGTATCAGTCCTAGACATTAGGCAGGGACCTAAAGAG
CCCTTCAAGGATTATGTGGACAGATTTTACAAGGCAATTAGAGCAGAACAAGCCTCAGGGGAAGTGAAACAATGG
ATGACAGAATCATTACTCATTCAAAATGCTAATCCAGATTGTAAGGTCATCCTGAAGGGCCTAGGAATGCACCCC
ACCCTTGAAGAAATGTTAACGGCTTGTCAGGGGGTAGGAGGCCCAAGCTACAAAGCAAAAGTAATGGCAGAAATG
ATGCAGACCATGCAAAATCAAAACATGGTGCAGCAGGGAGGTCCAAAAAGACAAAGACCCCCACTAAGATGTTAT
AATTGTGGAAAATTTGGCCATATGCAAAGACAATGTCCGGAACCAAGGAAAACAAAATGTCTAAAGTGTGGAAAA
TTGGGACACCTAGCAAAAGACTGCAGGGGACAGGTGAATTTTTTAGGGTATGGACGGTGGATGGGGGCAAAACCG
AGAAATTTTCCCGCCGCTACTCTTGGAGCGGAACCGAGTGCGCCTCCTCCACCGAGCGGCACCACCCCATACGAC
CCAGCAAAGAAGCTCCTGCAGCAATATGCAGAGAAAGGGAAACAACTGAGGGAGCAAAAGAGGAATCCACCGGCA
ATGAATCCGGATTGGACCGAGGGATATTCTTTGAACTCCCTCTTTGGAGAAGACCAATAAAGACAGTGTATATAG
AAGGGGTCCCCATTAAGGCACTGCTAGACACAGGGGCAGATGACACCATAATTAAAGAAAATGATTTACAATTAT
CAGGTCCATGGAGACCCAAAATTATAGGGGGCATAGGAGGAGGCCTTAATGTAAAAGAATATAACGACAGGGAAG
TAAAAATAGAAGATAAAATTTTGAGAGGAACAATATTGTTAGGAGCAACTCCCATTAATATAATAGGTAGAAATT
TGCTGGCCCCGGCAGGTGCCCGGTTAGTAATGGGACAATTATCAGAAAAAATTCCTGTCACACCTGTCAAATTGA
AGGAAGGGGCTCGGGGACCCTGTGTAAGACAATGGCCTCTCTCTAAAGAGAAGATTGAAGCTTTACAGGAAATAT
GTTCCCAATTAGAGCAGGAAGGAAAAATCAGTAGAGTAGGAGGAGAAAATGCATACAATACCCCAATATTTTGCA
TAAAGAAGAAGGACAAATCCCAGTGGAGGATGCTAGTAGACTTTAGAGAGTTAAATAAGGCAACCCAAGATTTCT
TTGAAGTGCAATTAGGGATACCCCACCCAGCAGGATTAAGAAAGATGAGACAGATAACAGTTTTAGATGTAGGAG
ACGCCTATTATTCCATACCATTGGATCCAAATTTTAGGAAATATACTGCTTTTACTATTCCCACAGTGAATAATC
AGGGACCCGGGATTAGGTATCAATTCAACTGTCTCCCGCAAGGGTGGAAAGGATCTCCTACAATCTTCCAAAATA
CAGCAGCATCCATTTTGGAGGAGATAAAAAGAAACTTGCCAGCACTAACCATTGTACAATACATGGATGATTTAT
GGGTAGGTTCTCAAGAAAATGAACACACCCATGACAAATTAGTAGAACAGTTAAGAACAAAATTACAAGCCTGGG
GCTTAGAAACCCCAGAAAAGAAGGTGCAAAAAGAACCACCTTATGAGTGGATGGGATACAAACTTTGGCCTCACA
AATGGGAACTAAGCAGAATACAACTGGAGGAAAAAGATGAATGGACTGTCAATGACATCCAGAAGTTAGTTGGGA
AACTAAATTGGGCAGCACAATTGTATCCAGGTCTTAGGACCAAGAATATATGCAAGTTAATTAGAGGAAAGAAAA
ATCTGTTAGAGCTAGTGACTTGGACACCTGAGGCAGAAGCTGAATATGCAGAAAATGCAGAGATTCTTAAAACAG
AACAGGAAGGAACCTATTACAAACCAGGAATACCTATTAGGGCAGCAGTACAGAAATTGGAAGGAGGACAGTGGA
GTTACCAATTCAAACAAGAAGGACAAGTCTTGAAAGTAGGAAAATACACCAAGCAAAAGAACACCCATACAAATG
AACTTCGCACATTAGCTGGTTTAGTGCAGAAGATTTGCAAAGAAGCTCTAGTTATTTGGGGGATATTACCAGTTC
TAGAACTCCCGATAGAAAGAGAGGTATGGGAACAATGGTGGGCGGATTACTGGCAGGTAAGCTGGATTCCCGAAT
GGGATTTTGTCAGCACCCCACCTTTGCTCAAACTATGGTACACATTAACAAAAGAACCCATACCCAAGGAGGACG
TTTACTATGTAGATGGAGCATGCAACAGAAATTCAAAAGAAGGAAAAGCAGGATACATCTCACAATACGGAAAAC
AGAGAGTAGAAACATTAGAAAACACTACCAATCAGCAAGCAGAATTAACAGCTATAAAAATGGCTTTGGAAGACA
GTGGGCCTAATGTGAACATAGTAACAGACTCTCAATATGCAATGGGAATTTTGACAGCACAACCCACACAAAGTG
ATTCACCATTAGTAGAGCAAATTATAGCCTTAATGATACAAAAGCAACAAATATATTTGCAGTGGGTACCAGCAC
ATAAAGGAATAGGAGGAAATGAGGAGATAGATAAATTAGTGAGTAAAGGCATTAGAAGAGTTTTATTCTTAGAAA
AAATAGAAGAAGCTCAAGAAGAGCATGAAAGATATCATAATAATTGGAAAAACCTAGCAGATACATATGGGCTTC
CACAAATAGTAGCAAAAGAGATAGTGGCCATGTGTCCAAAATGTCAGATAAAGGGAGAACCAGTGCATGGACAAG
TGGATGCCTCACCTGGAACATGGCAGATGGATTGTACTCATCTAGAAGGAAAAGTAGTCATAGTTGCGGTCCATG
TAGCCAGTGGATTCATAGAAGCAGAAGTCATACCTAGGGAAACAGGAAAAGAAACGGCAAAGTTTCTATTAAAAA
TACTGAGTAGATGGCCTATAACACAGTTACACACAGACAATGGGCCTAACTTTACCTCCCAAGAAGTGGCAGCAA
TATGTTGGTGGGGAAAAATTGAACATACAACAGGTATACCATATAACCCCCAATCTCAAGGATCAATAGAAAGCA
TGAACAAACAATTAAAAGAGATAATTGGGAAAATAAGAGATGATTGCCAATATACAGAGACAGCAGTACTGATGG
CTTGCCATATTCACAATTTTAAAAGAAAGGGAGGAATAGGGGGACAGACTTCAGCAGAGAGACTAATTAATATAA
TAACAACACAATTAGAAATACAACATTTACAAACCAAAATTCAAAAAATTTTAAATTTTAGAGTCTACTACAGAG
AAGGGAGAGACCCTGTGTGGAAAGGACCAGCACAATTAATCTGGAAAGGGGAAGGAGCAGTGGTCCTCAAGGACG
GAAGTGACCTAAAGGTTGTACCAAGAAGGAAAGCTAAAATTATTAAGGATTATGAACCCAAACAAAGAGTGGGTA
ATGAGGGTGACGTGGAAGGTACCAGGGGATCTGATAACTAA
SEQ ID NO: 6
codon-optimised SIV gal-pol nucleic acid sequence (from pGM691)
ATGGGAGCTGCCACATCTGCCCTGAATAGACGGCAGCTGGACCAGTTCGAGAAGATCAGACTGCGGCCCAACGGC
AAGAAGAAGTACCAGATCAAGCACCTGATCTGGGCCGGCAAAGAGATGGAAAGATTCGGCCTGCACGAGCGGCTG
CTGGAAACCGAGGAAGGCTGCAAGAGAATTATCGAGGTGCTGTACCCTCTGGAACCTACCGGCTCTGAGGGCCTG
AAGTCCCTGTTCAATCTCGTGTGCGTGCTGTACTGCCTGCACAAAGAACAGAAAGTGAAGGACACCGAAGAGGCC
GTGGCCACAGTTAGACAGCACTGCCACCTGGTGGAAAAAGAGAAGTCCGCCACAGAGACAAGCAGCGGCCAGAAG
AAGAACGACAAGGGAATTGCTGCCCCTCCTGGCGGCAGCCAGAATTTTCCTGCTCAGCAGCAGGGAAACGCCTGG
GTGCACGTTCCACTGAGCCCTAGAACACTGAATGCCTGGGTCAAAGCCGTGGAAGAGAAGAAGTTTGGCGCCGAG
ATCGTGCCCATGTTCCAGGCTCTGTCTGAGGGCTGCACCCCTTACGACATCAACCAGATGCTGAACGTGCTGGGA
GATCACCAGGGCGCTCTGCAGATCGTGAAAGAGATCATCAACGAAGAGGCTGCCCAGTGGGACGTGACACATCCA
TTGCCTGCTGGACCTCTGCCAGCCGGACAACTGAGAGATCCTAGAGGCTCTGATATCGCCGGCACCACCAGCTCT
GTGCAAGAGCAGCTGGAATGGATCTACACCGCCAATCCTAGAGTGGACGTGGGCGCCATCTACAGAAGATGGATC
ATCCTGGGCCTGCAGAAATGCGTGAAGATGTACAACCCCGTGTCCGTGCTGGACATCAGACAGGGACCCAAAGAG
CCCTTCAAGGACTACGTGGACCGGTTCTATAAGGCCATTAGAGCCGAGCAGGCCAGCGGCGAAGTGAAGCAGTGG
ATGACAGAGAGCCTGCTGATCCAGAACGCCAATCCAGACTGCAAAGTGATCCTGAAAGGCCTGGGCATGCACCCC
ACACTGGAAGAGATGCTGACAGCCTGTCAAGGCGTTGGCGGCCCTTCTTACAAAGCCAAAGTGATGGCCGAGATG
ATGCAGACCATGCAGAACCAGAACATGGTGCAGCAAGGCGGCCCTAAGAGACAGAGGCCTCCTCTGAGATGCTAC
AACTGCGGCAAGTTCGGCCACATGCAGAGACAGTGTCCTGAGCCTAGGAAAACAAAATGTCTAAAGTGTGGAAAA
TTGGGACACCTAGCAAAAGACTGCAGGGGACAGGTGAATTTTTTAGGGTATGGACGGTGGATGGGGGCAAAACCG
AGAAATTTTCCCGCCGCTACTCTTGGAGCGGAACCGAGTGCGCCTCCTCCACCGAGCGGCACCACCCCATACGAC
CCAGCAAAGAAGCTCCTGCAGCAATATGCAGAGAAAGGGAAACAACTGAGGGAGCAAAAGAGGAATCCACCGGCA
ATGAATCCGGATTGGACCGAGGGATATTCTTTGAACTCCCTCTTTGGAGAAGACCAATAAAGACCGTGTACATCG
AGGGCGTGCCCATCAAGGCTCTGCTGGATACAGGCGCCGACGACACCATCATCAAAGAGAACGACCTGCAGCTGA
GCGGCCCTTGGAGGCCTAAGATCATTGGAGGAATCGGCGGAGGCCTGAACGTCAAAGAGTACAACGACCGGGAAG
TGAAGATCGAGGACAAGATCCTGAGGGGCACAATCCTGCTGGGCGCCACACCTATCAACATCATCGGCAGAAATC
TGCTGGCCCCTGCCGGCGCTAGACTGGTTATGGGACAGCTCTCTGAGAAGATCCCCGTGACACCCGTGAAGCTGA
AAGAAGGCGCTAGAGGACCTTGTGTGCGACAGTGGCCTCTGAGCAAAGAGAAGATTGAGGCCCTGCAAGAAATCT
GTAGCCAGCTGGAACAAGAGGGCAAGATCAGCAGAGTTGGCGGCGAGAACGCCTACAATACCCCTATCTTCTGCA
TCAAGAAAAAGGACAAGAGCCAGTGGCGGATGCTGGTGGACTTTAGAGAGCTGAACAAGGCTACCCAGGACTTCT
TCGAGGTGCAGCTGGGAATTCCTCATCCTGCCGGCCTGCGGAAGATGAGACAGATCACAGTGCTGGATGTGGGCG
ACGCCTACTACAGCATCCCTCTGGACCCCAACTTCAGAAAGTACACCGCCTTCACAATCCCCACCGTGAACAATC
AAGGCCCTGGCATCAGATACCAGTTCAACTGCCTGCCTCAAGGCTGGAAGGGCAGCCCCACCATTTTTCAGAATA
CCGCCGCCAGCATCCTGGAAGAAATCAAGAGAAACCTGCCTGCTCTGACCATCGTGCAGTACATGGACGATCTGT
GGGTCGGAAGCCAAGAGAATGAGCACACCCACGACAAGCTGGTGGAACAGCTGAGAACAAAGCTGCAGGCCTGGG
GCCTCGAAACCCCTGAGAAGAAGGTGCAGAAAGAACCTCCTTACGAGTGGATGGGCTACAAGCTGTGGCCTCACA
AGTGGGAGCTGAGCCGGATTCAGCTCGAAGAGAAGGACGAGTGGACCGTGAACGACATCCAGAAACTCGTGGGCA
AGCTGAATTGGGCAGCCCAGCTGTATCCCGGCCTGAGGACCAAGAACATCTGCAAGCTGATCCGGGGAAAGAAGA
ACCTGCTGGAACTGGTCACATGGACACCTGAGGCCGAGGCCGAATATGCCGAGAATGCCGAAATCCTGAAAACCG
AGCAAGAGGGGACCTACTACAAGCCTGGCATTCCAATCAGAGCTGCCGTGCAGAAACTGGAAGGCGGCCAGTGGT
CCTACCAGTTTAAGCAAGAAGGCCAGGTCCTGAAAGTGGGCAAGTACACCAAGCAGAAGAACACCCACACCAACG
AGCTGAGGACACTGGCTGGCCTGGTCCAGAAAATCTGCAAAGAGGCCCTGGTCATTTGGGGCATCCTGCCTGTTC
TGGAACTGCCCATTGAGCGGGAAGTGTGGGAACAGTGGTGGGCCGATTACTGGCAAGTGTCTTGGATCCCCGAGT
GGGACTTCGTGTCTACCCCTCCTCTGCTGAAACTGTGGTACACCCTGACAAAAGAGCCCATTCCTAAAGAGGACG
TCTACTACGTTGACGGCGCCTGCAACCGGAACTCCAAAGAAGGCAAGGCCGGCTACATCAGCCAGTACGGCAAGC
AGAGAGTGGAAACCCTGGAAAACACCACCAACCAGCAGGCCGAGCTGACCGCCATTAAGATGGCCCTGGAAGATA
GCGGCCCCAATGTGAACATCGTGACCGACTCTCAGTACGCCATGGGAATCCTGACAGCCCAGCCTACACAGAGCG
ATAGCCCTCTGGTTGAGCAGATCATTGCCCTGATGATTCAGAAGCAGCAAATCTACCTGCAGTGGGTGCCCGCTC
ACAAAGGCATCGGCGGAAACGAAGAGATCGATAAGCTGGTGTCCAAGGGAATCAGACGGGTGCTGTTCCTGGAAA
AGATTGAAGAGGCCCAAGAGGAACACGAGCGCTACCACAACAACTGGAAGAATCTGGCCGACACCTACGGACTGC
CCCAGATCGTGGCCAAAGAAATCGTGGCTATGTGCCCCAAGTGTCAGATCAAGGGCGAACCTGTGCACGGCCAAG
TGGATGCTTCTCCTGGCACATGGCAGATGGACTGTACCCACCTGGAAGGCAAAGTGGTCATCGTGGCTGTGCACG
TGGCCTCCGGCTTTATTGAGGCCGAAGTGATCCCCAGAGAGACAGGCAAAGAAACCGCCAAGTTCCTGCTGAAGA
TCCTGTCCAGATGGCCCATCACACAGCTGCACACCGACAACGGCCCTAACTTCACATCTCAAGAGGTGGCCGCCA
TCTGTTGGTGGGGAAAGATTGAGCACACAACCGGCATTCCCTACAATCCACAGAGCCAGGGCAGCATCGAGTCCA
TGAACAAGCAGCTCAAAGAGATTATCGGCAAGATCCGGGACGACTGCCAGTACACAGAAACAGCCGTGCTGATGG
CCTGTCACATCCACAACTTCAAGCGGAAAGGCGGCATCGGAGGACAGACATCTGCCGAGAGACTGATCAATATCA
TCACCACTCAGCTGGAAATCCAGCACCTCCAGACCAAGATCCAGAAGATTCTGAACTTCCGGGTGTACTACCGCG
AGGGCAGAGATCCTGTTTGGAAAGGCCCAGCACAGCTGATCTGGAAAGGCGAAGGTGCCGTGGTGCTGAAGGATG
GCTCTGATCTGAAGGTGGTGCCCAGACGGAAGGCCAAGATTATCAAGGATTACGAGCCCAAACAGCGCGTGGGCA
ATGAAGGCGACGTTGAGGGCACAAGAGGCAGCGACAATTGA
SEQ ID NO: 7 Plasmid as defined in Figure 1C (pDNA2a pGM691)
ATTGATTATTGACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGGGCGCGCGCCAGGCGGGGCGGGGGGGG
GCGAGGGGGGGGGGGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCGCGTTACTCCCACAGG
TGAGCGGGCGGGACGGCCCTTCTCCTCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGGAGCGGCTCGGGGGGTGCGTGCGTGT
GTGTGTGCGTGGGGAGCGCCGCGTGCGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGTGCGGGGCTCCGTACGGGGCGTGGCG
CGGGGCTCGCCGTGCCGGGGGGGGGGGGCGGCAGGTGGGGGTGCCGGGCGGGGGGGGGCCGCCTCGGGCCGGGG
AGGGCTCGGGGGAGGGGCGCGGCGGCCCCCGGAGCGCCGGCGGCTGTCGAGGCGCGGCGAGCCGCAGCCATTGCC
TTTTATGGTAATCGTGCGAGAGGGCGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCAGGAAGGAAATGGGGGGGGAGGGCC
TTCGTGCGTCGCCGCGCCGCCGTCCCCTTCTCCCTCTCCAGCCTCGGGGCTGTCCGCGGGGGGACGGCTGCCTTC
GGGGGGGACGGGGCAGGGCGGGGTTCGGCTTCTGGCGTGTGACCGGCGGCTCTAGAGCCTCTGCTAACCATGTTC
ATGCCTTCTTCTTTTTCCTACAGCTCCTGGGCAACGTGCTGGTTATTGTGCTGTCTCATCATTTTGGCAAAGAAT
TGCTCGAGCCACCATGGGAGCTGCCACATCTGCCCTGAATAGACGGCAGCTGGACCAGTTCGAGAAGATCAGACT
GCGGCCCAACGGCAAGAAGAAGTACCAGATCAAGCACCTGATCTGGGCCGGCAAAGAGATGGAAAGATTCGGCCT
GCACGAGCGGCTGCTGGAAACCGAGGAAGGCTGCAAGAGAATTATCGAGGTGCTGTACCCTCTGGAACCTACCGG
CTCTGAGGGCCTGAAGTCCCTGTTCAATCTCGTGTGCGTGCTGTACTGCCTGCACAAAGAACAGAAAGTGAAGGA
CACCGAAGAGGCCGTGGCCACAGTTAGACAGCACTGCCACCTGGTGGAAAAAGAGAAGTCCGCCACAGAGACAAG
CAGCGGCCAGAAGAAGAACGACAAGGGAATTGCTGCCCCTCCTGGCGGCAGCCAGAATTTTCCTGCTCAGCAGCA
GGGAAACGCCTGGGTGCACGTTCCACTGAGCCCTAGAACACTGAATGCCTGGGTCAAAGCCGTGGAAGAGAAGAA
GTTTGGCGCCGAGATCGTGCCCATGTTCCAGGCTCTGTCTGAGGGCTGCACCCCTTACGACATCAACCAGATGCT
GAACGTGCTGGGAGATCACCAGGGCGCTCTGCAGATCGTGAAAGAGATCATCAACGAAGAGGCTGCCCAGTGGGA
CGTGACACATCCATTGCCTGCTGGACCTCTGCCAGCCGGACAACTGAGAGATCCTAGAGGCTCTGATATCGCCGG
CACCACCAGCTCTGTGCAAGAGCAGCTGGAATGGATCTACACCGCCAATCCTAGAGTGGACGTGGGCGCCATCTA
CAGAAGATGGATCATCCTGGGCCTGCAGAAATGCGTGAAGATGTACAACCCCGTGTCCGTGCTGGACATCAGACA
GGGACCCAAAGAGCCCTTCAAGGACTACGTGGACCGGTTCTATAAGGCCATTAGAGCCGAGCAGGCCAGCGGCGA
AGTGAAGCAGTGGATGACAGAGAGCCTGCTGATCCAGAACGCCAATCCAGACTGCAAAGTGATCCTGAAAGGCCT
GGGCATGCACCCCACACTGGAAGAGATGCTGACAGCCTGTCAAGGCGTTGGCGGCCCTTCTTACAAAGCCAAAGT
GATGGCCGAGATGATGCAGACCATGCAGAACCAGAACATGGTGCAGCAAGGCGGCCCTAAGAGACAGAGGCCTCC
TCTGAGATGCTACAACTGCGGCAAGTTCGGCCACATGCAGAGACAGTGTCCTGAGCCTAGGAAAACAAAATGTCT
AAAGTGTGGAAAATTGGGACACCTAGCAAAAGACTGCAGGGGACAGGTGAATTTTTTAGGGTATGGACGGTGGAT
GGGGGCAAAACCGAGAAATTTTCCCGCCGCTACTCTTGGAGCGGAACCGAGTGCGCCTCCTCCACCGAGCGGCAC
CACCCCATACGACCCAGCAAAGAAGCTCCTGCAGCAATATGCAGAGAAAGGGAAACAACTGAGGGAGCAAAAGAG
GAATCCACCGGCAATGAATCCGGATTGGACCGAGGGATATTCTTTGAACTCCCTCTTTGGAGAAGACCAATAAAG
ACCGTGTACATCGAGGGCGTGCCCATCAAGGCTCTGCTGGATACAGGCGCCGACGACACCATCATCAAAGAGAAC
GACCTGCAGCTGAGCGGCCCTTGGAGGCCTAAGATCATTGGAGGAATCGGCGGAGGCCTGAACGTCAAAGAGTAC
AACGACCGGGAAGTGAAGATCGAGGACAAGATCCTGAGGGGCACAATCCTGCTGGGCGCCACACCTATCAACATC
ATCGGCAGAAATCTGCTGGCCCCTGCCGGCGCTAGACTGGTTATGGGACAGCTCTCTGAGAAGATCCCCGTGACA
CCCGTGAAGCTGAAAGAAGGCGCTAGAGGACCTTGTGTGCGACAGTGGCCTCTGAGCAAAGAGAAGATTGAGGCC
CTGCAAGAAATCTGTAGCCAGCTGGAACAAGAGGGCAAGATCAGCAGAGTTGGCGGCGAGAACGCCTACAATACC
CCTATCTTCTGCATCAAGAAAAAGGACAAGAGCCAGTGGCGGATGCTGGTGGACTTTAGAGAGCTGAACAAGGCT
ACCCAGGACTTCTTCGAGGTGCAGCTGGGAATTCCTCATCCTGCCGGCCTGCGGAAGATGAGACAGATCACAGTG
CTGGATGTGGGCGACGCCTACTACAGCATCCCTCTGGACCCCAACTTCAGAAAGTACACCGCCTTCACAATCCCC
ACCGTGAACAATCAAGGCCCTGGCATCAGATACCAGTTCAACTGCCTGCCTCAAGGCTGGAAGGGCAGCCCCACC
ATTTTTCAGAATACCGCCGCCAGCATCCTGGAAGAAATCAAGAGAAACCTGCCTGCTCTGACCATCGTGCAGTAC
ATGGACGATCTGTGGGTCGGAAGCCAAGAGAATGAGCACACCCACGACAAGCTGGTGGAACAGCTGAGAACAAAG
CTGCAGGCCTGGGGCCTCGAAACCCCTGAGAAGAAGGTGCAGAAAGAACCTCCTTACGAGTGGATGGGCTACAAG
CTGTGGCCTCACAAGTGGGAGCTGAGCCGGATTCAGCTCGAAGAGAAGGACGAGTGGACCGTGAACGACATCCAG
AAACTCGTGGGCAAGCTGAATTGGGCAGCCCAGCTGTATCCCGGCCTGAGGACCAAGAACATCTGCAAGCTGATC
CGGGGAAAGAAGAACCTGCTGGAACTGGTCACATGGACACCTGAGGCCGAGGCCGAATATGCCGAGAATGCCGAA
ATCCTGAAAACCGAGCAAGAGGGGACCTACTACAAGCCTGGCATTCCAATCAGAGCTGCCGTGCAGAAACTGGAA
GGCGGCCAGTGGTCCTACCAGTTTAAGCAAGAAGGCCAGGTCCTGAAAGTGGGCAAGTACACCAAGCAGAAGAAC
ACCCACACCAACGAGCTGAGGACACTGGCTGGCCTGGTCCAGAAAATCTGCAAAGAGGCCCTGGTCATTTGGGGC
ATCCTGCCTGTTCTGGAACTGCCCATTGAGCGGGAAGTGTGGGAACAGTGGTGGGCCGATTACTGGCAAGTGTCT
TGGATCCCCGAGTGGGACTTCGTGTCTACCCCTCCTCTGCTGAAACTGTGGTACACCCTGACAAAAGAGCCCATT
CCTAAAGAGGACGTCTACTACGTTGACGGCGCCTGCAACCGGAACTCCAAAGAAGGCAAGGCCGGCTACATCAGC
CAGTACGGCAAGCAGAGAGTGGAAACCCTGGAAAACACCACCAACCAGCAGGCCGAGCTGACCGCCATTAAGATG
GCCCTGGAAGATAGCGGCCCCAATGTGAACATCGTGACCGACTCTCAGTACGCCATGGGAATCCTGACAGCCCAG
CCTACACAGAGCGATAGCCCTCTGGTTGAGCAGATCATTGCCCTGATGATTCAGAAGCAGCAAATCTACCTGCAG
TGGGTGCCCGCTCACAAAGGCATCGGCGGAAACGAAGAGATCGATAAGCTGGTGTCCAAGGGAATCAGACGGGTG
CTGTTCCTGGAAAAGATTGAAGAGGCCCAAGAGGAACACGAGCGCTACCACAACAACTGGAAGAATCTGGCCGAC
ACCTACGGACTGCCCCAGATCGTGGCCAAAGAAATCGTGGCTATGTGCCCCAAGTGTCAGATCAAGGGCGAACCT
GTGCACGGCCAAGTGGATGCTTCTCCTGGCACATGGCAGATGGACTGTACCCACCTGGAAGGCAAAGTGGTCATC
GTGGCTGTGCACGTGGCCTCCGGCTTTATTGAGGCCGAAGTGATCCCCAGAGAGACAGGCAAAGAAACCGCCAAG
TTCCTGCTGAAGATCCTGTCCAGATGGCCCATCACACAGCTGCACACCGACAACGGCCCTAACTTCACATCTCAA
GAGGTGGCCGCCATCTGTTGGTGGGGAAAGATTGAGCACACAACCGGCATTCCCTACAATCCACAGAGCCAGGGC
AGCATCGAGTCCATGAACAAGCAGCTCAAAGAGATTATCGGCAAGATCCGGGACGACTGCCAGTACACAGAAACA
GCCGTGCTGATGGCCTGTCACATCCACAACTTCAAGCGGAAAGGCGGCATCGGAGGACAGACATCTGCCGAGAGA
CTGATCAATATCATCACCACTCAGCTGGAAATCCAGCACCTCCAGACCAAGATCCAGAAGATTCTGAACTTCCGG
GTGTACTACCGCGAGGGCAGAGATCCTGTTTGGAAAGGCCCAGCACAGCTGATCTGGAAAGGCGAAGGTGCCGTG
GTGCTGAAGGATGGCTCTGATCTGAAGGTGGTGCCCAGACGGAAGGCCAAGATTATCAAGGATTACGAGCCCAAA
CAGCGCGTGGGCAATGAAGGCGACGTTGAGGGCACAAGAGGCAGCGACAATTGAAATTCACTCCTCAGGTGCAGG
CTGCCTATCAGAAGGTGGTGGCTGGTGTGGCCAATGCCCTGGCTCACAAATACCACTGAGATCTTTTTCCCTCTG
CCAAAAATTATGGGGACATCATGAAGCCCCTTGAGCATCTGACTTCTGGCTAATAAAGGAAATTTATTTTCATTG
CAATAGTGTGTTGGAATTTTTTGTGTCTCTCACTCGGAAGGACATATGGGAGGGCAAATCATTTAAAACATCAGA
ATGAGTATTTGGTTTAGAGTTTGGCAACATATGCCCATATGCTGGCTGCCATGAACAAAGGTTGGCTATAAAGAG
GTCATCAGTATATGAAACAGCCCCCTGCTGTCCATTCCTTATTCCATAGAAAAGCCTTGACTTGAGGTTAGATTT
TTTTTATATTTTGTTTTGTGTTATTTTTTTCTTTAACATCCCTAAAATTTTCCTTACATGTTTTACTAGCCAGAT
TTTTCCTCCTCTCCTGACTACTCCCAGTCATAGCTGTCCCTCTTCTCTTATGGAGATCCCTCGACCTGCAGCCCA
AGCTTGGCGTAATCATGGTCATAGCTGTTTCCTGTGTGAAATTGTTATCCGCTCACAATTCCACACAACATACGA
GCCGGAAGCATAAAGTGTAAAGCCTGGGGTGCCTAATGAGTGAGCTAACTCACATTAATTGCGTTGCGCTCACTG
CCCGCTTTCCAGTCGGGAAACCTGTCGTGCCAGCGGATCCGCATCTCAATTAGTCAGCAACCATAGTCCCGCCCC
TAACTCCGCCCATCCCGCCCCTAACTCCGCCCAGTTCCGCCCATTCTCCGCCCCATGGCTGACTAATTTTTTTTA
TTTATGCAGAGGCCGAGGCCGCCTCGGCCTCTGAGCTATTCCAGAAGTAGTGAGGAGGCTTTTTTGGAGGCCTAG
GCTTTTGCAAAAAGCTAACTTGTTTATTGCAGCTTATAATGGTTACAAATAAAGCAATAGCATCACAAATTTCAC
AAATAAAGCATTTTTTTCACTGCATTCTAGTTGTGGTTTGTCCAAACTCATCAATGTATCTTATCATGTCTGTCC
GCTTCCTCGCTCACTGACTCGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCCTCGTGCGCTCT
CCTGTTCCGACCCTGCCGCTTACCGGATACCTGTCCGCCTTTCTCCCTTCGGGAAGCGTGGCGCTTTCTCATAGC
TCACGCTGTAGGTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTATCGCCACTGGCA
GCAGCCACTGGTAACAGGATTAGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGCAGCAGATTACGCGCAGA
AAAAAAGGATCTCAAGAAGATCCTTTGATCTTTTCTACGGGGTCTGACGCTCAGTGGAACGAAAACTCACGTTAA
GGGATTTTGGTCATGAGATTATCAAAAAGGATCTTCACCTAGATCCTTTTAAATTAAAAATGAAGTTTTAAATCA
ATCTAAAGTATATATGAGTAAACTTGGTCTGACAGTTAGAAAAACTCATCGAGCATCAAATGAAACTGCAATTTA
TTCATATCAGGATTATCAATACCATATTTTTGAAAAAGCCGTTTCTGTAATGAAGGAGAAAACTCACCGAGGCAG
TTCCATAGGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGGCAACAGC
TTATGCATTTCTTTCCAGACTTGTTCAACAGGCCAGCCATTACGCTCGTCATCAAAATCACTCGCATCAACCAAA
CCGTTATTCATTCGTGATTGCGCCTGAGCGAGACGAAATACGCGATCGCTGTTAAAAGGACAATTACAAACAGGA
ATCGAATGCAACCGGCGCAGGAACACTGCCAGCGCATCAACAATATTTTCACCTGAATCAGGATATTCTTCTAAT
ACCTGGAATGCTGTTTTTCCGGGGATCGCAGTGGTGAGTAACCATGCATCATCAGGAGTACGGATAAAATGCTTG
ATGGTCGGAAGAGGCATAAATTCCGTCAGCCAGTTTAGTCTGACCATCTCATCTGTAACATCATTGGCAACGCTA
CCTTTGCCATGTTTCAGAAACAACTCTGGCGCATCGGGCTTCCCATACAATCGATAGATTGTCGCACCTGATTGC
CCGACATTATCGCGAGCCCATTTATACCCATATAAATCAGCATCCATGTTGGAATTTAATCGCGGCCTAGAGCAA
GACGTTTCCCGTTGAATATGGCTCATAACACCCCTTGTATTACTGTTTATGTAAGCAGACAGTTTTATTGTTCAT
GATGATATATTTTTATCTTGTGCAATGTAACATCAGAGATTTTGAGACACAACAATTGGTCGAC
SEQ ID NO: 8 Plasmid as defined in FIG. 1A (pDNA1 pGM326)
GGTACCTCAATATTGGCCATTAGCCATATTATTCATTGGTTATATAGCATAAATCAATATTGGCTATTGGCCATT
GCATACGTTGTATCTATATCATAATATGTACATTTATATTGGCTCATGTCCAATATGACCGCCATGTTGGCATTG
ATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGCCCACTTGGC
AGTACATCAAGTGTATCATATGCCAAGTCCGCCCCCTATTGACGTCAATGACGGTAAATGGCCCGCCTGGCATTA
TGCCCAGTACATGACCTTACGGGACTTTCCTACTTGGCAGTACATCTACGTATTAGTCATCGCTATTACCATGGT
GATGCGGTTTTGGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTGCGATCGCCC
GCCCCGTTGACGCAAATGGGCGGTAGGCGTGTACGGTGGGAGGTCTATATAAGCAGAGCTCGCTGGCTTGTAACT
CAGTCTCTTACTAGGAGACCAGCTTGAGCCTGGGTGTTCGCTGGTTAGCCTAACCTGGTTGGCCACCAGGGGTAA
GGACTCCTTGGCTTAGAAAGCTAATAAACTTGCCTGCATTAGAGCTTATCTGAGTCAAGTGTCCTCATTGACGCC
TCACTCTCTTGAACGGGAATCTTCCTTACTGGGTTCTCTCTCTGACCCAGGCGAGAGAAACTCCAGCAGTGGCGC
CCGAACAGGGACTTGAGTGAGAGTGTAGGCACGTACAGCTGAGAAGGCGTCGGACGCGAAGGAAGCGCGGGGTGC
GACGCGACCAAGAAGGAGACTTGGTGAGTAGGCTTCTCGAGTGCCGGGAAAAAGCTCGAGCCTAGTTAGAGGACT
AGGAGAGGCCGTAGCCGTAACTACTCTGGGCAAGTAGGGCAGGCGGTGGGTACGCAATGGGGGCGGCTACCTCAG
CACTAAATAGGAGACAATTAGACCAATTTGAGAAAATACGACTTCGCCCGAACGGAAAGAAAAAGTACCAAATTA
AACATTTAATATGGGCAGGCAAGGAGATGGAGCGCTTCGGCCTCCATGAGAGGTTGTTGGAGACAGAGGAGGGGT
GTAAAAGAATCATAGAAGTCCTCTACCCCCTAGAACCAACAGGATCGGAGGGCTTAAAAAGTCTGTTCAATCTTG
TGTGCGTGCTATATTGCTTGCACAAGGAACAGAAAGTGAAAGACACAGAGGAAGCAGTAGCAACAGTAAGACAAC
ACTGCCATCTAGTGGAAAAAGAAAAAAGTGCAACAGAGACATCTAGTGGACAAAAGAAAAATGACAAGGGAATAG
CAGCGCCACCTGGTGGCAGTCAGAATTTTCCAGCGCAACAACAAGGAAATGCCTGGGTACATGTACCCTTGTCAC
CGCGCACCTTAAATGCGTGGGTAAAAGCAGTAGAGGAGAAAAAATTTGGAGCAGAAATAGTACCCATGTTTCAAG
CCCTATCGAATTCCCGTTTGTGCTAGGGTTCTTAGGCTTCTTGGGGGCTGCTGGAACTGCAATGGGAGCAGCGGC
GACAGCCCTGACGGTCCAGTCTCAGCATTTGCTTGCTGGGATACTGCAGCAGCAGAAGAATCTGCTGGCGGCTGT
GGAGGCTCAACAGCAGATGTTGAAGCTGACCATTTGGGGTGTTAAAAACCTCAATGCCCGCGTCACAGCCCTTGA
GAAGTACCTAGAGGATCAGGCACGACTAAACTCCTGGGGGTGCGCATGGAAACAAGTATGTCATACCACAGTGGA
GTGGCCCTGGACAAATCGGACTCCGGATTGGCAAAATATGACTTGGTTGGAGTGGGAAAGACAAATAGCTGATTT
GGAAAGCAACATTACGAGACAATTAGTGAAGGCTAGAGAACAAGAGGAAAAGAATCTAGATGCCTATCAGAAGTT
AACTAGTTGGTCAGATTTCTGGTCTTGGTTCGATTTCTCAAAATGGCTTAACATTTTAAAAATGGGATTTTTAGT
AATAGTAGGAATAATAGGGTTAAGATTACTTTACACAGTATATGGATGTATAGTGAGGGTTAGGCAGGGATATGT
TCCTCTATCTCCACAGATCCATATCCGCGGCAATTTTAAAAGAAAGGGAGGAATAGGGGGACAGACTTCAGCAGA
GAGACTAATTAATATAATAACAACACAATTAGAAATACAACATTTACAAACCAAAATTCAAAAAATTTTAAATTT
TAGAGCCGCGGAGATCTGTTACATAACTTATGGTAAATGGCCTGCCTGGCTGACTGCCCAATGACCCCTGCCCAA
TGATGTCAATAATGATGTATGTTCCCATGTAATGCCAATAGGGACTTTCCATTGATGTCAATGGGTGGAGTATTT
ATGGTAACTGCCCACTTGGCAGTACATCAAGTGTATCATATGCCAAGTATGCCCCCTATTGATGTCAATGATGGT
AAATGGCCTGCCTGGCATTATGCCCAGTACATGACCTTATGGGACTTTCCTACTTGGCAGTACATCTATGTATTA
GTCATTGCTATTACCATGGGAATTCACTAGTGGAGAAGAGCATGCTTGAGGGCTGAGTGCCCCTCAGTGGGCAGA
GAGCACATGGCCCACAGTCCCTGAGAAGTTGGGGGGAGGGGTGGGCAATTGAACTGGTGCCTAGAGAAGGTGGGG
CTTGGGTAAACTGGGAAAGTGATGTGGTGTACTGGCTCCACCTTTTTCCCCAGGGTGGGGGAGAACCATATATAA
GTGCAGTAGTCTCTGTGAACATTCAAGCTTCTGCCTTCTCCCTCCTGTGAGTTTGCTAGCCACCATGCAGAGAAG
CCCTCTGGAGAAGGCCTCTGTGGTGAGCAAGCTGTTCTTCAGCTGGACCAGGCCCATCCTGAGGAAGGGCTACAG
GCAGAGACTGGAGCTGTCTGACATCTACCAGATCCCCTCTGTGGACTCTGCTGACAACCTGTCTGAGAAGCTGGA
GAGGGAGTGGGATAGAGAGCTGGCCAGCAAGAAGAACCCCAAGCTGATCAATGCCCTGAGGAGATGCTTCTTCTG
GAGATTCATGTTCTATGGCATCTTCCTGTACCTGGGGGAAGTGACCAAGGCTGTGCAGCCTCTGCTGCTGGGCAG
AATCATTGCCAGCTATGACCCTGACAACAAGGAGGAGAGGAGCATTGCCATCTACCTGGGCATTGGCCTGTGCCT
GCTGTTCATTGTGAGGACCCTGCTGCTGCACCCTGCCATCTTTGGCCTGCACCACATTGGCATGCAGATGAGGAT
TGCCATGTTCAGCCTGATCTACAAGAAAACCCTGAAGCTGTCCAGCAGAGTGCTGGACAAGATCAGCATTGGCCA
GCTGGTGAGCCTGCTGAGCAACAACCTGAACAAGTTTGATGAGGGCCTGGCCCTGGCCCACTTTGTGTGGATTGC
CCCTCTGCAGGTGGCCCTGCTGATGGGCCTGATTTGGGAGCTGCTGCAGGCCTCTGCCTTTTGTGGCCTGGGCTT
CCTGATTGTGCTGGCCCTGTTTCAGGCTGGCCTGGGCAGGATGATGATGAAGTACAGGGACCAGAGGGCAGGCAA
GATCAGTGAGAGGCTGGTGATCACCTCTGAGATGATTGAGAACATCCAGTCTGTGAAGGCCTACTGTTGGGAGGA
AGCTATGGAGAAGATGATTGAAAACCTGAGGCAGACAGAGCTGAAGCTGACCAGGAAGGCTGCCTATGTGAGATA
CTTCAACAGCTCTGCCTTCTTCTTCTCTGGCTTCTTTGTGGTGTTCCTGTCTGTGCTGCCCTATGCCCTGATCAA
GGGGATCATCCTGAGAAAGATTTTCACCACCATCAGCTTCTGCATTGTGCTGAGGATGGCTGTGACCAGACAGTT
CCCCTGGGCTGTGCAGACCTGGTATGACAGCCTGGGGGCCATCAACAAGATCCAGGACTTCCTGCAGAAGCAGGA
GTACAAGACCCTGGAGTACAACCTGACCACCACAGAAGTGGTGATGGAGAATGTGACAGCCTTCTGGGAGGAGGG
CTTTGGGGAGCTGTTTGAGAAGGCCAAGCAGAACAACAACAACAGAAAGACCAGCAATGGGGATGACTCCCTGTT
CTTCTCCAACTTCTCCCTGCTGGGCACACCTGTGCTGAAGGACATCAACTTCAAGATTGAGAGGGGGCAGCTGCT
GGCTGTGGCTGGATCTACAGGGGCTGGCAAGACCAGCCTGCTGATGATGATCATGGGGGAGCTGGAGCCTTCTGA
GGGCAAGATCAAGCACTCTGGCAGGATCAGCTTTTGCAGCCAGTTCAGCTGGATCATGCCTGGCACCATCAAGGA
GAACATCATCTTTGGAGTGAGCTATGATGAGTACAGATACAGGAGTGTGATCAAGGCCTGCCAGCTGGAGGAGGA
CATCAGCAAGTTTGCTGAGAAGGACAACATTGTGCTGGGGGAGGGAGGCATTACACTGTCTGGGGGCCAGAGAGC
CAGAATCAGCCTGGCCAGGGCTGTGTACAAGGATGCTGACCTGTACCTGCTGGACTCCCCCTTTGGCTACCTGGA
TGTGCTGACAGAGAAGGAGATTTTTGAGAGCTGTGTGTGCAAGCTGATGGCCAACAAGACCAGAATCCTGGTGAC
CAGCAAGATGGAGCACCTGAAGAAGGCTGACAAGATCCTGATCCTGCATGAGGGCAGCAGCTACTTCTATGGGAC
CTTCTCTGAGCTGCAGAACCTGCAGCCTGACTTCAGCTCTAAGCTGATGGGCTGTGACAGCTTTGACCAGTTCTC
TGCTGAGAGGAGGAACAGCATCCTGACAGAGACCCTGCACAGATTCAGCCTGGAGGGAGATGCCCCTGTGAGCTG
GACAGAGACCAAGAAGCAGAGCTTCAAGCAGACAGGGGAGTTTGGGGAGAAGAGGAAGAACTCCATCCTGAACCC
CATCAACAGCATCAGGAAGTTCAGCATTGTGCAGAAAACCCCCCTGCAGATGAATGGCATTGAGGAAGATTCTGA
TGAGCCCCTGGAGAGGAGACTGAGCCTGGTGCCTGATTCTGAGCAGGGAGAGGCCATCCTGCCTAGGATCTCTGT
GATCAGCACAGGCCCTACACTGCAGGCCAGAAGGAGGCAGTCTGTGCTGAACCTGATGACCCACTCTGTGAACCA
GGGCCAGAACATCCACAGGAAAACCACAGCCTCCACCAGGAAAGTGAGCCTGGCCCCTCAGGCCAATCTGACAGA
GCTGGACATCTACAGCAGGAGGCTGTCTCAGGAGACAGGCCTGGAGATTTCTGAGGAGATCAATGAGGAGGACCT
GAAAGAGTGCTTCTTTGATGACATGGAGAGCATCCCTGCTGTGACCACCTGGAACACCTACCTGAGATACATCAC
AGTGCACAAGAGCCTGATCTTTGTGCTGATCTGGTGCCTGGTGATCTTCCTGGCTGAAGTGGCTGCCTCTCTGGT
GGTGCTGTGGCTGCTGGGAAACACCCCACTGCAGGACAAGGGCAACAGCACCCACAGCAGGAACAACAGCTATGC
TGTGATCATCACCTCCACCTCCAGCTACTATGTGTTCTACATCTATGTGGGAGTGGCTGATACCCTGCTGGCTAT
GGGCTTCTTTAGAGGCCTGCCCCTGGTGCACACACTGATCACAGTGAGCAAGATCCTCCACCACAAGATGCTGCA
CTCTGTGCTGCAGGCTCCTATGAGCACCCTGAATACCCTGAAGGCTGGGGGCATCCTGAACAGATTCTCCAAGGA
TATTGCCATCCTGGATGACCTGCTGCCTCTCACCATCTTTGACTTCATCCAGCTGCTGCTGATTGTGATTGGGGC
CATTGCTGTGGTGGCAGTGCTGCAGCCCTACATCTTTGTGGCCACAGTGCCTGTGATTGTGGCCTTCATCATGCT
GAGGGCCTACTTTCTGCAGACCTCCCAGCAGCTGAAGCAGCTGGAGTCTGAGGGCAGAAGCCCCATCTTCACCCA
CCTGGTGACAAGCCTGAAGGGCCTGTGGACCCTGAGAGCCTTTGGCAGGCAGCCCTACTTTGAGACCCTGTTCCA
CAAGGCCCTGAACCTGCACACAGCCAACTGGTTCCTCTACCTGTCCACCCTGAGATGGTTCCAGATGAGAATTGA
GATGATCTTTGTCATCTTCTTCATTGCTGTGACCTTCATCAGCATTCTGACCACAGGAGAGGGAGAGGGCAGAGT
GGGCATTATCCTGACCCTGGCCATGAACATCATGAGCACACTGCAGTGGGCAGTGAACAGCAGCATTGATGTGGA
CAGCCTGATGAGGAGTGTGAGCAGAGTGTTCAAGTTCATTGATATGCCCACAGAGGGCAAGCCTACCAAGAGCAC
CAAGCCCTACAAGAATGGCCAGCTGAGCAAAGTGATGATCATTGAGAACAGCCATGTGAAGAAGGATGATATCTG
GCCCAGTGGAGGCCAGATGACAGTGAAGGACCTGACAGCCAAGTACACAGAGGGGGGCAATGCTATCCTGGAGAA
CATCTCCTTCAGCATCTCCCCTGGCCAGAGAGTGGGACTGCTGGGAAGAACAGGCTCTGGCAAGTCTACCCTGCT
GTCTGCCTTCCTGAGGCTGCTGAACACAGAGGGAGAGATCCAGATTGATGGAGTGTCCTGGGACAGCATCACACT
GCAGCAGTGGAGGAAGGCCTTTGGTGTGATCCCCCAGAAAGTGTTCATCTTCAGTGGCACCTTCAGGAAGAACCT
GGACCCCTATGAGCAGTGGTCTGACCAGGAGATTTGGAAAGTGGCTGATGAAGTGGGCCTGAGAAGTGTGATTGA
GCAGTTCCCTGGCAAGCTGGACTTTGTCCTGGTGGATGGGGGCTGTGTGCTGAGCCATGGCCACAAGCAGCTGAT
GTGCCTGGCCAGATCAGTGCTGAGCAAGGCCAAGATCCTGCTGCTGGATGAGCCTTCTGCCCACCTGGATCCTGT
GACCTACCAGATCATCAGGAGGACCCTCAAGCAGGCCTTTGCTGACTGCACAGTCATCCTGTGTGAGCACAGGAT
TGAGGCCATGCTGGAGTGCCAGCAGTTCCTGGTGATTGAGGAGAACAAAGTGAGGCAGTATGACAGCATCCAGAA
GCTGCTGAATGAGAGGAGCCTGTTCAGGCAGGCCATCAGCCCCTCTGATAGAGTGAAGCTGTTCCCCCACAGGAA
CAGCTCCAAGTGCAAGAGCAAGCCCCAGATTGCTGCCCTGAAGGAGGAGACAGAGGAGGAAGTGCAGGACACCAG
GCTGTGAGGGCCCAATCAACCTCTGGATTACAAAATTTGTGAAAGATTGACTGGTATTCTTAACTATGTTGCTCC
TTTTACGCTATGTGGATACGCTGCTTTAATGCCTTTGTATCATGCTATTGCTTCCCGTATGGCTTTCATTTTCTC
CTCCTTGTATAAATCCTGGTTGCTGTCTCTTTATGAGGAGTTGTGGCCCGTTGTCAGGCAACGTGGCGTGGTGTG
CACTGTGTTTGCTGACGCAACCCCCACTGGTTGGGGCATTGCCACCACCTGTCAGCTCCTTTCCGGGACTTTCGC
TTTCCCCCTCCCTATTGCCACGGCGGAACTCATCGCCGCCTGCCTTGCCCGCTGCTGGACAGGGGCTCGGCTGTT
GGGCACTGACAATTCCGTGGTGTTGTCGGGGAAATCATCGTCCTTTCCTTGGCTGCTCGCCTGTGTTGCCACCTG
GATTCTGCGCGGGACGTCCTTCTGCTACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGTCGGATCTCCCTTTGGGCCGCCTCCCC
GCAAGCTTCGCACTTTTTAAAAGAAAAGGGAGGACTGGATGGGATTTATTACTCCGATAGGACGCTGGCTTGTAA
CTCAGTCTCTTACTAGGAGACCAGCTTGAGCCTGGGTGTTCGCTGGTTAGCCTAACCTGGTTGGCCACCAGGGGT
AAGGACTCCTTGGCTTAGAAAGCTAATAAACTTGCCTGCATTAGAGCTCTTACGCGTCCCGGGCTCGAGATCCGC
ATCTCAATTAGTCAGCAACCATAGTCCCGCCCCTAACTCCGCCCATCCCGCCCCTAACTCCGCCCAGTTCCGCCC
ATTCTCCGCCCCATGGCTGACTAATTTTTTTTATTTATGCAGAGGCCGAGGCCGCCTCGGCCTCTGAGCTATTCC
AGAAGTAGTGAGGAGGCTTTTTTGGAGGCCTAGGCTTTTGCAAAAAGCTAACTTGTTTATTGCAGCTTATAATGG
TTACAAATAAAGCAATAGCATCACAAATTTCACAAATAAAGCATTTTTTTCACTGCATTCTAGTTGTGGTTTGTC
CAAACTCATCAATGTATCTTATCATGTCTGTCCGCTTCCTCGCTCACTGACTCGCTGCGCTCGGTCGTTCGGCTG
CGGCGAGCGGTATCAGCTCACTCAAAGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATCTTTTCTACGGGG
TCTGACGCTCAGTGGAACGAAAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAACTTGGTCTGACAGTTAGAAA
AACTCATCGAGCATCAAATGAAACTGCAATTTATTCATATCAGGATTATCAATACCATATTTTTGAAAAAGCCGT
TTCTGTAATGAAGGAGAAAACTCACCGAGGCAGTTCCATAGGATGGCAAGATCCTGGTATCGGTCTGCGATTCCG
ACTCGTCCAACATCAATACAACCTATTAATTTCCCCTCGTCAAAAATAAGGTTATCAAGTGAGAAATCACCATGA
GTGACGACTGAATCCGGTGAGAATGGCAACAGCTTATGCATTTCTTTCCAGACTTGTTCAACAGGCCAGCCATTA
CGCTCGTCATCAAAATCACTCGCATCAACCAAACCGTTATTCATTCGTGATTGCGCCTGAGCGAGACGAAATACG
CGATCGCTGTTAAAAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTTCCGGGGATCGCAGTGGTGAGTAAC
CATGCATCATCAGGAGTACGGATAAAATGCTTGATGGTCGGAAGAGGCATAAATTCCGTCAGCCAGTTTAGTCTG
ACCATCTCATCTGTAACATCATTGGCAACGCTACCTTTGCCATGTTTCAGAAACAACTCTGGCGCATCGGGCTTC
CCATACAATCGATAGATTGTCGCACCTGATTGCCCGACATTATCGCGAGCCCATTTATACCCATATAAATCAGCA
TCCATGTTGGAATTTAATCGCGGCCTAGAGCAAGACGTTTCCCGTTGAATATGGCTCATAACACCCCTTGTATTA
CTGTTTATGTAAGCAGACAGTTTTATTGTTCATGATGATATATTTTTATCTTGTGCAATGTAACATCAGAGATTT
TGAGACACAACAATTGGTCGACGGATCC
SEQ ID NO: 9 Plasmid as defined in FIG. 1B (pDNA1 pGM830)
GGTACCTCAATATTGGCCATTAGCCATATTATTCATTGGTTATATAGCATAAATCAATATTGGCTATTGGCCATT
GCATACGTTGTATCTATATCATAATATGTACATTTATATTGGCTCATGTCCAATATGACCGCCATGTTGGCATTG
ATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGCCCACTTGGC
AGTACATCAAGTGTATCATATGCCAAGTCCGCCCCCTATTGACGTCAATGACGGTAAATGGCCCGCCTGGCATTA
TGCCCAGTACATGACCTTACGGGACTTTCCTACTTGGCAGTACATCTACGTATTAGTCATCGCTATTACCATGGT
GATGCGGTTTTGGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTGCGATCGCCC
GCCCCGTTGACGCAAATGGGCGGTAGGCGTGTACGGTGGGAGGTCTATATAAGCAGAGCTCGCTGGCTTGTAACT
CAGTCTCTTACTAGGAGACCAGCTTGAGCCTGGGTGTTCGCTGGTTAGCCTAACCTGGTTGGCCACCAGGGGTAA
GGACTCCTTGGCTTAGAAAGCTAATAAACTTGCCTGCATTAGAGCTTATCTGAGTCAAGTGTCCTCATTGACGCC
TCACTCTCTTGAACGGGAATCTTCCTTACTGGGTTCTCTCTCTGACCCAGGCGAGAGAAACTCCAGCAGTGGCGC
CCGAACAGGGACTTGAGTGAGAGTGTAGGCACGTACAGCTGAGAAGGCGTCGGACGCGAAGGAAGCGCGGGGTGC
GACGCGACCAAGAAGGAGACTTGGTGAGTAGGCTTCTCGAGTGCCGGGAAAAAGCTCGAGCCTAGTTAGAGGACT
AGGAGAGGCCGTAGCCGTAACTACTCTGGGCAAGTAGGGCAGGCGGTGGGTACGCAATTGGGGGCGGCTACCTCA
GCACTAAATAGGAGACAATTAGACCAATTTGAGAAAATACGACTTCGCCCGAACGGAAAGAAAAAGTACCAAATT
AAACATTTAATATTGGGCAGGCAAGGAGATTGGAGCGCTTCGGCCTCCATGAGAGGTTGTTGGAGACAGAGGAGG
GGTGTAAAAGAATCATAGAAGTCCTCTACCCCCTAGAACCAACAGGATCGGAGGGCTTAAAAAGTCTGTTCAATC
TTGTGTGCGTGCTATATTGCTTGCACAAGGAACAGAAAGTGAAAGACACAGAGGAAGCAGTAGCAACAGTAAGAC
AACACTGCCATCTAGTGGAAAAAGAAAAAAGTGCAACAGAGACATCTAGTGGACAAAAGAAAAATGACAAGGGAA
TAGCAGCGCCACCTGGTGGCAGTCAGAATTTTCCAGCGCAACAACAAGGAAATTGCCTGGGTACATGTACCCTTG
TCACCGCGCACCTTAAATGCGTGGGTAAAAGCAGTAGAGGAGAAAAAATTTGGAGCAGAAATAGTACCCATGTTT
CAAGCCCTATCGCCTGCAGGCCGTTTGTGCTAGGGTTCTTAGGCTTCTTGGGGGCTGCTGGAACTGCATTGGGAG
CAGCGGCGACAGCCCTGACGGTCCAGTCTCAGCATTTGCTTGCTGGGATACTGCAGCAGCAGAAGAATCTGCTGG
CGGCTGTGGAGGCTCAACAGCAGATGTTGAAGCTGACCATTTGGGGTGTTAAAAACCTCAATGCCCGCGTCACAG
CCCTTGAGAAGTACCTAGAGGATCAGGCACGACTAAACTCCTGGGGGTGCGCATGGAAACAAGTATGTCATACCA
CAGTGGAGTGGCCCTGGACAAATCGGACTCCGGATTGGCAAAATAAGACTTGGTTGGAGTGGGAAAGACAAATAG
CTGATTTGGAAAGCAACATTACGAGACAATTAGTGAAGGCTAGAGAACAAGAGGAAAAGAATCTAGATGCCTATC
AGAAGTTAACTAGTTGGTCAGATTTCTGGTCTTGGTTCGATTTCTCAAAATGGCTTAACATTTTAAAAAAGGGAT
TTTTAGTAATAGTAGGAATAATAGGGTTAAGATTACTTTACACAGTATATGGATGTATAGTGAGGGTTAGGCAGG
GATATGTTCCTCTATCTCCACAGATCCATATAAAGCGGCAATTTTAAAAGAAAGGGAGGAATAGGGGGACAGACT
TCAGCAGAGAGACTAATTAATATAATAACAACACAATTAGAAATACAACATTTACAAACCAAAATTCAAAAAATT
TTAAATTTTAGAGCCGCGGAGATCTGTTACATAACTTATGGTAAATGGCCTGCCTGGCTGACTGCCCAATGACCC
CTGCCCAATGATGTCAATAATGATGTATGTTCCCATGTAATGCCAATAGGGACTTTCCATTGATGTCAATGGGTG
GAGTATTTATGGTAACTGCCCACTTGGCAGTACATCAAGTGTATCATATGCCAAGTATGCCCCCTATTGATGTCA
ATGATGGTAAATGGCCTGCCTGGCATTATGCCCAGTACATGACCTTATGGGACTTTCCTACTTGGCAGTACATCT
ATGTATTAGTCATTGCTATTACCATGGGAATTCACTAGTGGAGAAGAGCATGCTTGAGGGCTGAGTGCCCCTCAG
TGGGCAGAGAGCACATGGCCCACAGTCCCTGAGAAGTTGGGGGGAGGGGTGGGCAATTGAACTGGTGCCTAGAGA
AGGTGGGGCTTGGGTAAACTGGGAAAGTGATGTGGTGTACTGGCTCCACCTTTTTCCCCAGGGTGGGGGAGAACC
ATATATAAGTGCAGTAGTCTCTGTGAACATTCAAGCTTCTGCCTTCTCCCTCCTGTGAGTTTGCTAGCCACCATG
CAGAGAAGCCCTCTGGAGAAGGCCTCTGTGGTGAGCAAGCTGTTCTTCAGCTGGACCAGGCCCATCCTGAGGAAG
GGCTACAGGCAGAGACTGGAGCTGTCTGACATCTACCAGATCCCCTCTGTGGACTCTGCTGACAACCTGTCTGAG
AAGCTGGAGAGGGAGTGGGATAGAGAGCTGGCCAGCAAGAAGAACCCCAAGCTGATCAATGCCCTGAGGAGATGC
TTCTTCTGGAGATTCATGTTCTATGGCATCTTCCTGTACCTGGGGGAAGTGACCAAGGCTGTGCAGCCTCTGCTG
CTGGGCAGAATCATTGCCAGCTATGACCCTGACAACAAGGAGGAGAGGAGCATTGCCATCTACCTGGGCATTGGC
CTGTGCCTGCTGTTCATTGTGAGGACCCTGCTGCTGCACCCTGCCATCTTTGGCCTGCACCACATTGGCATGCAG
ATGAGGATTGCCATGTTCAGCCTGATCTACAAGAAAACCCTGAAGCTGTCCAGCAGAGTGCTGGACAAGATCAGC
ATTGGCCAGCTGGTGAGCCTGCTGAGCAACAACCTGAACAAGTTTGATGAGGGCCTGGCCCTGGCCCACTTTGTG
TGGATTGCCCCTCTGCAGGTGGCCCTGCTGATGGGCCTGATTTGGGAGCTGCTGCAGGCCTCTGCCTTTTGTGGC
CTGGGCTTCCTGATTGTGCTGGCCCTGTTTCAGGCTGGCCTGGGCAGGATGATGATGAAGTACAGGGACCAGAGG
GCAGGCAAGATCAGTGAGAGGCTGGTGATCACCTCTGAGATGATTGAGAACATCCAGTCTGTGAAGGCCTACTGT
TGGGAGGAAGCTATGGAGAAGATGATTGAAAACCTGAGGCAGACAGAGCTGAAGCTGACCAGGAAGGCTGCCTAT
GTGAGATACTTCAACAGCTCTGCCTTCTTCTTCTCTGGCTTCTTTGTGGTGTTCCTGTCTGTGCTGCCCTATGCC
CTGATCAAGGGGATCATCCTGAGAAAGATTTTCACCACCATCAGCTTCTGCATTGTGCTGAGGATGGCTGTGACC
AGACAGTTCCCCTGGGCTGTGCAGACCTGGTATGACAGCCTGGGGGCCATCAACAAGATCCAGGACTTCCTGCAG
AAGCAGGAGTACAAGACCCTGGAGTACAACCTGACCACCACAGAAGTGGTGATGGAGAATGTGACAGCCTTCTGG
GAGGAGGGCTTTGGGGAGCTGTTTGAGAAGGCCAAGCAGAACAACAACAACAGAAAGACCAGCAATGGGGATGAC
TCCCTGTTCTTCTCCAACTTCTCCCTGCTGGGCACACCTGTGCTGAAGGACATCAACTTCAAGATTGAGAGGGGG
CAGCTGCTGGCTGTGGCTGGATCTACAGGGGCTGGCAAGACCAGCCTGCTGATGATGATCATGGGGGAGCTGGAG
CCTTCTGAGGGCAAGATCAAGCACTCTGGCAGGATCAGCTTTTGCAGCCAGTTCAGCTGGATCATGCCTGGCACC
ATCAAGGAGAACATCATCTTTGGAGTGAGCTATGATGAGTACAGATACAGGAGTGTGATCAAGGCCTGCCAGCTG
GAGGAGGACATCAGCAAGTTTGCTGAGAAGGACAACATTGTGCTGGGGGAGGGAGGCATTACACTGTCTGGGGGC
CAGAGAGCCAGAATCAGCCTGGCCAGGGCTGTGTACAAGGATGCTGACCTGTACCTGCTGGACTCCCCCTTTGGC
TACCTGGATGTGCTGACAGAGAAGGAGATTTTTGAGAGCTGTGTGTGCAAGCTGATGGCCAACAAGACCAGAATC
CTGGTGACCAGCAAGATGGAGCACCTGAAGAAGGCTGACAAGATCCTGATCCTGCATGAGGGCAGCAGCTACTTC
TATGGGACCTTCTCTGAGCTGCAGAACCTGCAGCCTGACTTCAGCTCTAAGCTGATGGGCTGTGACAGCTTTGAC
CAGTTCTCTGCTGAGAGGAGGAACAGCATCCTGACAGAGACCCTGCACAGATTCAGCCTGGAGGGAGATGCCCCT
GTGAGCTGGACAGAGACCAAGAAGCAGAGCTTCAAGCAGACAGGGGAGTTTGGGGAGAAGAGGAAGAACTCCATC
CTGAACCCCATCAACAGCATCAGGAAGTTCAGCATTGTGCAGAAAACCCCCCTGCAGATGAATGGCATTGAGGAA
GATTCTGATGAGCCCCTGGAGAGGAGACTGAGCCTGGTGCCTGATTCTGAGCAGGGAGAGGCCATCCTGCCTAGG
ATCTCTGTGATCAGCACAGGCCCTACACTGCAGGCCAGAAGGAGGCAGTCTGTGCTGAACCTGATGACCCACTCT
GTGAACCAGGGCCAGAACATCCACAGGAAAACCACAGCCTCCACCAGGAAAGTGAGCCTGGCCCCTCAGGCCAAT
CTGACAGAGCTGGACATCTACAGCAGGAGGCTGTCTCAGGAGACAGGCCTGGAGATTTCTGAGGAGATCAATGAG
GAGGACCTGAAAGAGTGCTTCTTTGATGACATGGAGAGCATCCCTGCTGTGACCACCTGGAACACCTACCTGAGA
TACATCACAGTGCACAAGAGCCTGATCTTTGTGCTGATCTGGTGCCTGGTGATCTTCCTGGCTGAAGTGGCTGCC
TCTCTGGTGGTGCTGTGGCTGCTGGGAAACACCCCACTGCAGGACAAGGGCAACAGCACCCACAGCAGGAACAAC
AGCTATGCTGTGATCATCACCTCCACCTCCAGCTACTATGTGTTCTACATCTATGTGGGAGTGGCTGATACCCTG
CTGGCTATGGGCTTCTTTAGAGGCCTGCCCCTGGTGCACACACTGATCACAGTGAGCAAGATCCTCCACCACAAG
ATGCTGCACTCTGTGCTGCAGGCTCCTATGAGCACCCTGAATACCCTGAAGGCTGGGGGCATCCTGAACAGATTC
TCCAAGGATATTGCCATCCTGGATGACCTGCTGCCTCTCACCATCTTTGACTTCATCCAGCTGCTGCTGATTGTG
ATTGGGGCCATTGCTGTGGTGGCAGTGCTGCAGCCCTACATCTTTGTGGCCACAGTGCCTGTGATTGTGGCCTTC
ATCATGCTGAGGGCCTACTTTCTGCAGACCTCCCAGCAGCTGAAGCAGCTGGAGTCTGAGGGCAGAAGCCCCATC
TTCACCCACCTGGTGACAAGCCTGAAGGGCCTGTGGACCCTGAGAGCCTTTGGCAGGCAGCCCTACTTTGAGACC
CTGTTCCACAAGGCCCTGAACCTGCACACAGCCAACTGGTTCCTCTACCTGTCCACCCTGAGATGGTTCCAGATG
AGAATTGAGATGATCTTTGTCATCTTCTTCATTGCTGTGACCTTCATCAGCATTCTGACCACAGGAGAGGGAGAG
GGCAGAGTGGGCATTATCCTGACCCTGGCCATGAACATCATGAGCACACTGCAGTGGGCAGTGAACAGCAGCATT
GATGTGGACAGCCTGATGAGGAGTGTGAGCAGAGTGTTCAAGTTCATTGATATGCCCACAGAGGGCAAGCCTACC
AAGAGCACCAAGCCCTACAAGAATGGCCAGCTGAGCAAAGTGATGATCATTGAGAACAGCCATGTGAAGAAGGAT
GATATCTGGCCCAGTGGAGGCCAGATGACAGTGAAGGACCTGACAGCCAAGTACACAGAGGGGGGCAATGCTATC
CTGGAGAACATCTCCTTCAGCATCTCCCCTGGCCAGAGAGTGGGACTGCTGGGAAGAACAGGCTCTGGCAAGTCT
ACCCTGCTGTCTGCCTTCCTGAGGCTGCTGAACACAGAGGGAGAGATCCAGATTGATGGAGTGTCCTGGGACAGC
ATCACACTGCAGCAGTGGAGGAAGGCCTTTGGTGTGATCCCCCAGAAAGTGTTCATCTTCAGTGGCACCTTCAGG
AAGAACCTGGACCCCTATGAGCAGTGGTCTGACCAGGAGATTTGGAAAGTGGCTGATGAAGTGGGCCTGAGAAGT
GTGATTGAGCAGTTCCCTGGCAAGCTGGACTTTGTCCTGGTGGATGGGGGCTGTGTGCTGAGCCATGGCCACAAG
CAGCTGATGTGCCTGGCCAGATCAGTGCTGAGCAAGGCCAAGATCCTGCTGCTGGATGAGCCTTCTGCCCACCTG
GATCCTGTGACCTACCAGATCATCAGGAGGACCCTCAAGCAGGCCTTTGCTGACTGCACAGTCATCCTGTGTGAG
CACAGGATTGAGGCCATGCTGGAGTGCCAGCAGTTCCTGGTGATTGAGGAGAACAAAGTGAGGCAGTATGACAGC
ATCCAGAAGCTGCTGAATGAGAGGAGCCTGTTCAGGCAGGCCATCAGCCCCTCTGATAGAGTGAAGCTGTTCCCC
CACAGGAACAGCTCCAAGTGCAAGAGCAAGCCCCAGATTGCTGCCCTGAAGGAGGAGACAGAGGAGGAAGTGCAG
GACACCAGGCTGTGAGGGCCCAATCAACCTCTGGATTACAAAATTTGTGAAAGATTGACTGGTATTCTTAACTAT
GTTGCTCCTTTTACGCTATGTGGATACGCTGCTTTAATGCCTTTGTATCATGCTATTGCTTCCCGTATGGCTTTC
ATTTTCTCCTCCTTGTATAAATCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACCACCTGTCAGCTCCTTTCCGGG
ACTTTCGCTTTCCCCCTCCCTATTGCCACGGCGGAACTCATCGCCGCCTGCCTTGCCCGCTGCTGGACAGGGGCT
CGGCTGTTGGGCACTGACAATTCCGTGGTGTTGTCGGGGAAATCATCGTCCTTTCCTTGGCTGCTCGCCTGTGTT
GCCACCTGGATTCTGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGATCTCCCTTTGGGCC
GCCTCCCCGCAAGCTTCGCACTTTTTAAAAGAAAAGGGAGGACTGGATGGGATTTATTACTCCGATAGGACGCTG
GCTTGTAACTCAGTCTCTTACTAGGAGACCAGCTTGAGCCTGGGTGTTCGCTGGTTAGCCTAACCTGGTTGGCCA
CCAGGGGTAAGGACTCCTTGGCTTAGAAAGCTAATAAACTTGCCTGCATTAGAGCTCTTACGCGTCCCGGGCTCG
AGATCCGCATCTCAATTAGTCAGCAACCATAGTCCCGCCCCTAACTCCGCCCATCCCGCCCCTAACTCCGCCCAG
TTCCGCCCATTCTCCGCCCCATGGCTGACTAATTTTTTTTATTTATGCAGAGGCCGAGGCCGCCTCGGCCTCTGA
GCTATTCCAGAAGTAGTGAGGAGGCTTTTTTGGAGGCCTAGGCTTTTGCAAAAAGCTAACTTGTTTATTGCAGCT
TATAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTGTCCGCTTCCTCGCTCACTGACTCGCTGCGCTCGGTCG
TTCGGCTGCGGCGAGCGGTATCAGCTCACTCAAAGGCGGTAATACGGTTATCCACAGAATCAGGGGATAACGCAG
GAAAGAACATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGACTATAAA
GATACCAGGCGTTTCCCCCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCTCAGTTCGGTGTAGGTCG
TTCGCTCCAAGCTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGAGATTATCAAAAAGGATCT
TCACCTAGATCCTTTTAAATTAAAAATGAAGTTTTAAATCAATCTAAAGTATATATGAGTAAACTTGGTCTGACA
GTTAGAAAAACTCATCGAGCATCAAATGAAACTGCAATTTATTCATATCAGGATTATCAATACCATATTTTTGAA
AAAGCCGTTTCTGTAATGAAGGAGAAAACTCACCGAGGCAGTTCCATAGGATGGCAAGATCCTGGTATCGGTCTG
CGATTCCGACTCGTCCAACATCAATACAACCTATTAATTTCCCCTCGTCAAAAATAAGGTTATCAAGTGAGAAAT
CACCATGAGTGACGACTGAATCCGGTGAGAATGGCAACAGCTTATGCATTTCTTTCCAGACTTGTTCAACAGGCC
AGCCATTACGCTCGTCATCAAAATCACTCGCATCAACCAAACCGTTATTCATTCGTGATTGCGCCTGAGCGAGAC
GAAATACGCGATCGCTGTTAAAAGGACAATTACAAACAGGAATCGAATGCAACCGGCGCAGGAACACTGCCAGCG
CATCAACAATATTTTCACCTGAATCAGGATATTCTTCTAATACCTGGAATGCTGTTTTTCCGGGGATCGCAGTGG
TGAGTAACCATGCATCATCAGGAGTACGGATAAAATGCTTGATGGTCGGAAGAGGCATAAATTCCGTCAGCCAGT
TTAGTCTGACCATCTCATCTGTAACATCATTGGCAACGCTACCTTTGCCATGTTTCAGAAACAACTCTGGCGCAT
CGGGCTTCCCATACAATCGATAGATTGTCGCACCTGATTGCCCGACATTATCGCGAGCCCATTTATACCCATATA
AATCAGCATCCATGTTGGAATTTAATCGCGGCCTAGAGCAAGACGTTTCCCGTTGAATATGGCTCATAACACCCC
TTGTATTACTGTTTATGTAAGCAGACAGTTTTATTGTTCATGATGATATATTTTTATCTTGTGCAATGTAACATC
AGAGATTTTGAGACACAACAATTGGTCGACGGATCC
SEQ ID NO: 10 Plasmid as defined in FIG. 1D (pDNA2a pGM297)
ATTGATTATTGACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGGGCGCGCGCCAGGCGGGGCGGGGGGGG
GCGAGGGGCGGGGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCGCGTTACTCCCACAGG
TGAGCGGGGGGACGGCCCTTCTCCTCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGGAGCGGCTCGGGGGGTGCGTGCGTGT
GTGTGTGCGTGGGGAGCGCCGCGTGCGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGTGCGGGGCTCCGTACGGGGCGTGGCG
CGGGGCTCGCCGTGCCGGGGGGGGGGTGGCGGCAGGTGGGGGTGCCGGGGGGGGGGGGGCCGCCTCGGGCCGGGG
AGGGCTCGGGGGAGGGGCGCGGCGGCCCCCGGAGCGCCGGCGGCTGTCGAGGCGCGGCGAGCCGCAGCCATTGCC
TTTTATGGTAATCGTGCGAGAGGGCGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCAGGAAGGAAATGGGGGGGGAGGGCC
TTCGTGCGTCGCCGCGCCGCCGTCCCCTTCTCCCTCTCCAGCCTCGGGGCTGTCCGCGGGGGGACGGCTGCCTTC
GGGGGGGACGGGGCAGGGCGGGGTTCGGCTTCTGGCGTGTGACCGGCGGCTCTAGAGCCTCTGCTAACCATGTTC
ATGCCTTCTTCTTTTTCCTACAGCTCCTGGGCAACGTGCTGGTTATTGTGCTGTCTCATCATTTTGGCAAAGAAT
TGCTCGAGACTAGTGACTTGGTGAGTAGGCTTCGAGCCTAGTTAGAGGACTAGGAGAGGCCGTAGCCGTAACTAC
TCTGGGCAAGTAGGGCAGGCGGTGGGTACGCAATGGGGGCGGCTACCTCAGCACTAAATAGGAGACAATTAGACC
AATTTGAGAAAATACGACTTCGCCCGAACGGAAAGAAAAAGTACCAAATTAAACATTTAATATGGGCAGGCAAGG
AGATGGAGCGCTTCGGCCTCCATGAGAGGTTGTTGGAGACAGAGGAGGGGTGTAAAAGAATCATAGAAGTCCTCT
ACCCCCTAGAACCAACAGGATCGGAGGGCTTAAAAAGTCTGTTCAATCTTGTGTGCGTACTATATTGCTTGCACA
AGGAACAGAAAGTGAAAGACACAGAGGAAGCAGTAGCAACAGTAAGACAACACTGCCATCTAGTGGAAAAAGAAA
AAAGTGCAACAGAGACATCTAGTGGACAAAAGAAAAATGACAAGGGAATAGCAGCGCCACCTGGTGGCAGTCAGA
ATTTTCCAGCGCAACAACAAGGAAATGCCTGGGTACATGTACCCTTGTCACCGCGCACCTTAAATGCGTGGGTAA
AAGCAGTAGAGGAGAAAAAATTTGGAGCAGAAATAGTACCCATGTTTCAAGCCCTATCAGAAGGCTGCACACCCT
ATGACATTAATCAGATGCTTAATGTGCTAGGAGATCATCAAGGGGCATTACAAATAGTGAAAGAGATCATTAATG
AAGAAGCAGCCCAGTGGGATGTAACACACCCACTACCCGCAGGACCCCTACCAGCAGGACAGCTCAGGGACCCTC
GCGGCTCAGATATAGCAGGGACCACCAGCTCAGTACAAGAACAGTTAGAATGGATCTATACTGCTAACCCCCGGG
TAGATGTAGGTGCCATCTACCGGAGATGGATTATTCTAGGACTTCAAAAGTGTGTCAAAATGTACAACCCAGTAT
CAGTCCTAGACATTAGGCAGGGACCTAAAGAGCCCTTCAAGGATTATGTGGACAGATTTTACAAGGCAATTAGAG
CAGAACAAGCCTCAGGGGAAGTGAAACAATGGATGACAGAATCATTACTCATTCAAAATGCTAATCCAGATTGTA
AGGTCATCCTGAAGGGCCTAGGAATGCACCCCACCCTTGAAGAAATGTTAACGGCTTGTCAGGGGGTAGGAGGCC
CAAGCTACAAAGCAAAAGTAATGGCAGAAATGATGCAGACCATGCAAAATCAAAACATGGTGCAGCAGGGAGGTC
CAAAAAGACAAAGACCCCCACTAAGATGTTATAATTGTGGAAAATTTGGCCATATGCAAAGACAATGTCCGGAAC
CAAGGAAAACAAAATGTCTAAAGTGTGGAAAATTGGGACACCTAGCAAAAGACTGCAGGGGACAGGTGAATTTTT
TAGGGTATGGACGGTGGATGGGGGCAAAACCGAGAAATTTTCCCGCCGCTACTCTTGGAGCGGAACCGAGTGCGC
CTCCTCCACCGAGCGGCACCACCCCATACGACCCAGCAAAGAAGCTCCTGCAGCAATATGCAGAGAAAGGGAAAC
AACTGAGGGAGCAAAAGAGGAATCCACCGGCAATGAATCCGGATTGGACCGAGGGATATTCTTTGAACTCCCTCT
TTGGAGAAGACCAATAAAGACAGTGTATATAGAAGGGGTCCCCATTAAGGCACTGCTAGACACAGGGGCAGATGA
CACCATAATTAAAGAAAATGATTTACAATTATCAGGTCCATGGAGACCCAAAATTATAGGGGGCATAGGAGGAGG
CCTTAATGTAAAAGAATATAACGACAGGGAAGTAAAAATAGAAGATAAAATTTTGAGAGGAACAATATTGTTAGG
AGCAACTCCCATTAATATAATAGGTAGAAATTTGCTGGCCCCGGCAGGTGCCCGGTTAGTAATGGGACAATTATC
AGAAAAAATTCCTGTCACACCTGTCAAATTGAAGGAAGGGGCTCGGGGACCCTGTGTAAGACAATGGCCTCTCTC
TAAAGAGAAGATTGAAGCTTTACAGGAAATATGTTCCCAATTAGAGCAGGAAGGAAAAATCAGTAGAGTAGGAGG
AGAAAATGCATACAATACCCCAATATTTTGCATAAAGAAGAAGGACAAATCCCAGTGGAGGATGCTAGTAGACTT
TAGAGAGTTAAATAAGGCAACCCAAGATTTCTTTGAAGTGCAATTAGGGATACCCCACCCAGCAGGATTAAGAAA
GATGAGACAGATAACAGTTTTAGATGTAGGAGACGCCTATTATTCCATACCATTGGATCCAAATTTTAGGAAATA
TACTGCTTTTACTATTCCCACAGTGAATAATCAGGGACCCGGGATTAGGTATCAATTCAACTGTCTCCCGCAAGG
GTGGAAAGGATCTCCTACAATCTTCCAAAATACAGCAGCATCCATTTTGGAGGAGATAAAAAGAAACTTGCCAGC
ACTAACCATTGTACAATACATGGATGATTTATGGGTAGGTTCTCAAGAAAATGAACACACCCATGACAAATTAGT
AGAACAGTTAAGAACAAAATTACAAGCCTGGGGCTTAGAAACCCCAGAAAAGAAGGTGCAAAAAGAACCACCTTA
TGAGTGGATGGGATACAAACTTTGGCCTCACAAATGGGAACTAAGCAGAATACAACTGGAGGAAAAAGATGAATG
GACTGTCAATGACATCCAGAAGTTAGTTGGGAAACTAAATTGGGCAGCACAATTGTATCCAGGTCTTAGGACCAA
GAATATATGCAAGTTAATTAGAGGAAAGAAAAATCTGTTAGAGCTAGTGACTTGGACACCTGAGGCAGAAGCTGA
ATATGCAGAAAATGCAGAGATTCTTAAAACAGAACAGGAAGGAACCTATTACAAACCAGGAATACCTATTAGGGC
AGCAGTACAGAAATTGGAAGGAGGACAGTGGAGTTACCAATTCAAACAAGAAGGACAAGTCTTGAAAGTAGGAAA
ATACACCAAGCAAAAGAACACCCATACAAATGAACTTCGCACATTAGCTGGTTTAGTGCAGAAGATTTGCAAAGA
AGCTCTAGTTATTTGGGGGATATTACCAGTTCTAGAACTCCCGATAGAAAGAGAGGTATGGGAACAATGGTGGGC
GGATTACTGGCAGGTAAGCTGGATTCCCGAATGGGATTTTGTCAGCACCCCACCTTTGCTCAAACTATGGTACAC
ATTAACAAAAGAACCCATACCCAAGGAGGACGTTTACTATGTAGATGGAGCATGCAACAGAAATTCAAAAGAAGG
AAAAGCAGGATACATCTCACAATACGGAAAACAGAGAGTAGAAACATTAGAAAACACTACCAATCAGCAAGCAGA
ATTAACAGCTATAAAAATGGCTTTGGAAGACAGTGGGCCTAATGTGAACATAGTAACAGACTCTCAATATGCAAT
GGGAATTTTGACAGCACAACCCACACAAAGTGATTCACCATTAGTAGAGCAAATTATAGCCTTAATGATACAAAA
GCAACAAATATATTTGCAGTGGGTACCAGCACATAAAGGAATAGGAGGAAATGAGGAGATAGATAAATTAGTGAG
TAAAGGCATTAGAAGAGTTTTATTCTTAGAAAAAATAGAAGAAGCTCAAGAAGAGCATGAAAGATATCATAATAA
TTGGAAAAACCTAGCAGATACATATGGGCTTCCACAAATAGTAGCAAAAGAGATAGTGGCCATGTGTCCAAAATG
TCAGATAAAGGGAGAACCAGTGCATGGACAAGTGGATGCCTCACCTGGAACATGGCAGATGGATTGTACTCATCT
AGAAGGAAAAGTAGTCATAGTTGCGGTCCATGTAGCCAGTGGATTCATAGAAGCAGAAGTCATACCTAGGGAAAC
AGGAAAAGAAACGGCAAAGTTTCTATTAAAAATACTGAGTAGATGGCCTATAACACAGTTACACACAGACAATGG
GCCTAACTTTACCTCCCAAGAAGTGGCAGCAATATGTTGGTGGGGAAAAATTGAACATACAACAGGTATACCATA
TAACCCCCAATCTCAAGGATCAATAGAAAGCATGAACAAACAATTAAAAGAGATAATTGGGAAAATAAGAGATGA
TTGCCAATATACAGAGACAGCAGTACTGATGGCTTGCCATATTCACAATTTTAAAAGAAAGGGAGGAATAGGGGG
ACAGACTTCAGCAGAGAGACTAATTAATATAATAACAACACAATTAGAAATACAACATTTACAAACCAAAATTCA
AAAAATTTTAAATTTTAGAGTCTACTACAGAGAAGGGAGAGACCCTGTGTGGAAAGGACCAGCACAATTAATCTG
GAAAGGGGAAGGAGCAGTGGTCCTCAAGGACGGAAGTGACCTAAAGGTTGTACCAAGAAGGAAAGCTAAAATTAT
TAAGGATTATGAACCCAAACAAAGAGTGGGTAATGAGGGTGACGTGGAAGGTACCAGGGGATCTGATAACTAAAT
GGCAGGGAATAGTCAGATATTGGATGAGACAAAGAAATTTGAAATGGAACTATTATATGCATCAGCTGGCGGCCG
CGAATTCACTAGTGATTCCCGTTTGTGCTAGGGTTCTTAGGCTTCTTGGGGGCTGCTGGAACTGCAATGGGAGCA
GCGGCGACAGCCCTGACGGTCCAGTCTCAGCATTTGCTTGCTGGGATACTGCAGCAGCAGAAGAATCTGCTGGCG
GCTGTGGAGGCTCAACAGCAGATGTTGAAGCTGACCATTTGGGGTGTTAAAAACCTCAATGCCCGCGTCACAGCC
CTTGAGAAGTACCTAGAGGATCAGGCACGACTAAACTCCTGGGGGTGCGCATGGAAACAAGTATGTCATACCACA
GTGGAGTGGCCCTGGACAAATCGGACTCCGGATTGGCAAAATATGACTTGGTTGGAGTGGGAAAGACAAATAGCT
GATTTGGAAAGCAACATTACGAGACAATTAGTGAAGGCTAGAGAACAAGAGGAAAAGAATCTAGATGCCTATCAG
AAGTTAACTAGTTGGTCAGATTTCTGGTCTTGGTTCGATTTCTCAAAATGGCTTAACATTTTAAAAATGGGATTT
TTAGTAATAGTAGGAATAATAGGGTTAAGATTACTTTACACAGTATATGGATGTATAGTGAGGGTTAGGCAGGGA
TATGTTCCTCTATCTCCACAGATCCATATCCAATCGAATTCCCGCGGCCGCAATTCACTCCTCAGGTGCAGGCTG
CCTATCAGAAGGTGGTGGCTGGTGTGGCCAATGCCCTGGCTCACAAATACCACTGAGATCTTTTTCCCTCTGCCA
AAAATTATGGGGACATCATGAAGCCCCTTGAGCATCTGACTTCTGGCTAATAAAGGAAATTTATTTTCATTGCAA
TAGTGTGTTGGAATTTTTTGTGTCTCTCACTCGGAAGGACATATGGGAGGGCAAATCATTTAAAACATCAGAATG
AGTATTTGGTTTAGAGTTTGGCAACATATGCCCATATGCTGGCTGCCATGAACAAAGGTTGGCTATAAAGAGGTC
ATCAGTATATGAAACAGCCCCCTGCTGTCCATTCCTTATTCCATAGAAAAGCCTTGACTTGAGGTTAGATTTTTT
TTATATTTTGTTTTGTGTTATTTTTTTCTTTAACATCCCTAAAATTTTCCTTACATGTTTTACTAGCCAGATTTT
TCCTCCTCTCCTGACTACTCCCAGTCATAGCTGTCCCTCTTCTCTTATGGAGATCCCTCGACCTGCAGCCCAAGC
TTGGCGTAATCATGGTCATAGCTGTTTCCTGTGTGAAATTGTTATCCGCTCACAATTCCACACAACATACGAGCC
GGAAGCATAAAGTGTAAAGCCTGGGGTGCCTAATGAGTGAGCTAACTCACATTAATTGCGTTGCGCTCACTGCCC
GCTTTCCAGTCGGGAAACCTGTCGTGCCAGCGGATCCGCATCTCAATTAGTCAGCAACCATAGTCCCGCCCCTAA
CTCCGCCCATCCCGCCCCTAACTCCGCCCAGTTCCGCCCATTCTCCGCCCCATGGCTGACTAATTTTTTTTATTT
ATGCAGAGGCCGAGGCCGCCTCGGCCTCTGAGCTATTCCAGAAGTAGTGAGGAGGCTTTTTTGGAGGCCTAGGCT
TTTGCAAAAAGCTAACTTGTTTATTGCAGCTTATAATGGTTACAAATAAAGCAATAGCATCACAAATTTCACAAA
TAAAGCATTTTTTTCACTGCATTCTAGTTGTGGTTTGTCCAAACTCATCAATGTATCTTATCATGTCTGTCCGCT
TCCTCGCTCACTGACTCGCTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGTGGCCTAACTAC
GGCTACACTAGAAGAACAGTATTTGGTATCTGCGCTCTGCTGAAGCCAGTTACCTTCGGAAAAAGAGTTGGTAGC
TCTTGATCCGGCAAACAAACCACCGCTGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTCAGTGGAACGAAAACTCACGTTAAGGG
ATTTTGGTCATGAGATTATCAAAAAGGATCTTCACCTAGATCCTTTTAAATTAAAAATGAAGTTTTAAATCAATC
TAAAGTATATATGAGTAAACTTGGTCTGACAGTTAGAAAAACTCATCGAGCATCAAATGAAACTGCAATTTATTC
ATATCAGGATTATCAATACCATATTTTTGAAAAAGCCGTTTCTGTAATGAAGGAGAAAACTCACCGAGGCAGTTC
CATAGGATGGCAAGATCCTGGTATCGGTCTGCGATTCCGACTCGTCCAACATCAATACAACCTATTAATTTCCCC
TCGTCAAAAATAAGGTTATCAAGTGAGAAATCACCATGAGTGACGACTGAATCCGGTGAGAATGGCAACAGCTTA
TGCATTTCTTTCCAGACTTGTTCAACAGGCCAGCCATTACGCTCGTCATCAAAATCACTCGCATCAACCAAACCG
TTATTCATTCGTGATTGCGCCTGAGCGAGACGAAATACGCGATCGCTGTTAAAAGGACAATTACAAACAGGAATC
GAATGCAACCGGCGCAGGAACACTGCCAGCGCATCAACAATATTTTCACCTGAATCAGGATATTCTTCTAATACC
TGGAATGCTGTTTTTCCGGGGATCGCAGTGGTGAGTAACCATGCATCATCAGGAGTACGGATAAAATGCTTGATG
GTCGGAAGAGGCATAAATTCCGTCAGCCAGTTTAGTCTGACCATCTCATCTGTAACATCATTGGCAACGCTACCT
TTGCCATGTTTCAGAAACAACTCTGGCGCATCGGGCTTCCCATACAATCGATAGATTGTCGCACCTGATTGCCCG
ACATTATCGCGAGCCCATTTATACCCATATAAATCAGCATCCATGTTGGAATTTAATCGCGGCCTAGAGCAAGAC
GTTTCCCGTTGAATATGGCTCATAACACCCCTTGTATTACTGTTTATGTAAGCAGACAGTTTTATTGTTCATGAT
GATATATTTTTATCTTGTGCAATGTAACATCAGAGATTTTGAGACACAACAATTGGTCGAC
SEQ ID NO: 11 Plasmid as defined in FIG. 1E (pDNA2b pGM299)
TCAATATTGGCCATTAGCCATATTATTCATTGGTTATATAGCATAAATCAATATTGGCTATTGGCCATTGCATAC
GTTGTATCTATATCATAATATGTACATTTATATTGGCTCATGTCCAATATGACCGCCATGTTGGCATTGATTATT
GACTAGTTATTAATAGTAATCAATTACGGGGTCATTAGTTCATAGCCCATATATGGAGTTCCGCGTTACATAACT
TACGGTAAATGGCCCGCCTGGCTGACCGCCCAACGACCCCCGCCCATTGACGTCAATAATGACGTATGTTCCCAT
AGTAACGCCAATAGGGACTTTCCATTGACGTCAATGGGTGGAGTATTTACGGTAAACTGCCCACTTGGCAGTACA
TCAAGTGTATCATATGCCAAGTCCGCCCCCTATTGACGTCAATGACGGTAAATGGCCCGCCTGGCATTATGCCCA
GTACATGACCTTACGGGACTTTCCTACTTGGCAGTACATCTACGTATTAGTCATCGCTATTACCATGGTGATGCG
GTTTTGGCAGTACACCAATGGGCGTGGATAGCGGTTTGACTCACGGGGATTTCCAAGTCTCCACCCCATTGACGT
CAATGGGAGTTTGTTTTGGCACCAAAATCAACGGGACTTTCCAAAATGTCGTAATAACCCCGCCCCGTTGACGCA
AATGGGCGGTAGGCGTGTACGGTGGGAGGTCTATATAAGCAGAGCTCGTTTAGTGAACCGTCAGATCACTAGAAG
CTTTATTGCGGTAGTTTATCACAGTTAAATTGCTAACGCAGTCAGTGCTTCTGACACAACAGTCTCGAACTTAAG
CTGCAGAAGTTGGTCGTGAGGCACTGGGCAGGTAAGTATCAAGGTTACAAGACAGGTTTAAGGAGACCAATAGAA
ACTGGGCTTGTCGAGACAGAGAAGACTCTTGCGTTTCTGATAGGCACCTATTGGTCTTACTGACATCCACTTTGC
CTTTCTCTCCACAGGTGTCCACTCCCAGTTCAATTACAGCTCTTAAGGCTAGAGTACTTAATACGACTCACTATA
GGCTAGCCTCGAGAATTCGATTATGCCCCTAGGACCAGAAGAAAGAAGATTGCTTCGCTTGATTTGGCTCCTTTA
CAGCACCAATCCATATCCACCAAGTGGGGAAGGGACGGCCAGACAACGCCGACGAGCCAGGAGAAGGTGGAGACA
ACAGCAGGATCAAATTAGAGTCTTGGTAGAAAGACTCCAAGAGCAGGTGTATGCAGTTGACCGCCTGGCTGACGA
GGCTCAACACTTGGCTATACAACAGTTGCCTGACCCTCCTCATTCAGCTTAGAATCACTAGTGAATTCACGCGTG
GTACCTCTAGAGTCGACCCGGGCGGCCGCTTCGAGCAGACATGATAAGATACATTGATGAGTTTGGACAAACCAC
AACTAGAATGCAGTGAAAAAAATGCTTTATTTGTGAAATTTGTGATGCTATTGCTTTATTTGTAACCATTATAAG
CTGCAATAAACAAGTTAACAACAACAATTGCATTCATTTTATGTTTCAGGTTCAGGGGGAGATGTGGGAGGTTTT
TTAAAGCAAGTAAAACCTCTACAAATGTGGTAAAATCGATAAGGATCCGTCGACCAATTGTTGTGTCTCAAAATC
TCTGATGTTACATTGCACAAGATAAAAATATATCATCATGAACAATAAAACTGTCTGCTTACATAAACAGTAATA
CAAGGGGTGTTATGAGCCATATTCAACGGGAAACGTCTTGCTCTAGGCCGCGATTAAATTCCAACATGGATGCTG
ATTTATATGGGTATAAATGGGCTCGCGATAATGTCGGGCAATCAGGTGCGACAATCTATCGATTGTATGGGAAGC
CCGATGCGCCAGAGTTGTTTCTGAAACATGGCAAAGGTAGCGTTGCCAATGATGTTACAGATGAGATGGTCAGAC
TAAACTGGCTGACGGAATTTATGCCTCTTCCGACCATCAAGCATTTTATCCGTACTCCTGATGATGCATGGTTAC
TCACCACTGCGATCCCCGGAAAAACAGCATTCCAGGTATTAGAAGAATATCCTGATTCAGGTGAAAATATTGTTG
ATGCGCTGGCAGTGTTCCTGCGCCGGTTGCATTCGATTCCTGTTTGTAATTGTCCTTTTAACAGCGATCGCGTAT
TTCGTCTCGCTCAGGCGCAATCACGAATGAATAACGGTTTGGTTGATGCGAGTGATTTTGATGACGAGCGTAATG
GCTGGCCTGTTGAACAAGTCTGGAAAGAAATGCATAAGCTGTTGCCATTCTCACCGGATTCAGTCGTCACTCATG
GTGATTTCTCACTTGATAACCTTATTTTTGACGAGGGGAAATTAATAGGTTGTATTGATGTTGGACGAGTCGGAA
TCGCAGACCGATACCAGGATCTTGCCATCCTATGGAACTGCCTCGGTGAGTTTTCTCCTTCATTACAGAAACGGC
TTTTTCAAAAATATGGTATTGATAATCCTGATATGAATAAATTGCAGTTTCATTTGATGCTCGATGAGTTTTTCT
AACTGTCAGACCAAGTTTACTCATATATACTTTAGATTGATTTAAAACTTCATTTTTAATTTAAAAGGATCTAGG
TGAAGATCCTTTTTGATAATCTCATGACCAAAATCCCTTAACGTGAGTTTTCGTTCCACTGAGCGTCAGACCCCG
TAGAAAAGATCAAAGGATCTTCTTGAGATCCTTTTTTTCTGCGCGTAATCTGCTGCTTGCAAACAAAAAAACCAC
CGCTACCAGCGGTGGTTTGTTTGCCGGATCAAGAGCTACCAACTCTTTTTCCGAAGGTAACTGGCTTCAGCAGAG
CGCAGATACCAAATACTGTTCTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTTGCTGGCCTTTTGCTCACATGGCTC
GACAGATCT
SEQ ID NO: 12 Plasmid as defined in FIG. 1F (pDNA3a pGM301)
ATTGATTATTGACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGGGCGCGCGCCAGGCGGGGCGGGGCGGG
GCGAGGGGCGGGGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCGCGTTACTCCCACAGG
TGAGCGGGCGGGACGGCCCTTCTCCTCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGGAGCGGCTCGGGGGGTGCGTGCGTGT
GTGTGTGCGTGGGGAGCGCCGCGTGCGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGTGCGGGGCTCCGTACGGGGCGTGGCG
CGGGGCTCGCCGTGCCGGGCGGGGGGGGGCGGCAGGTGGGGGTGCCGGGGGGGGCGGGGCCGCCTCGGGCCGGGG
AGGGCTCGGGGGAGGGGCGCGGCGGCCCCCGGAGCGCCGGCGGCTGTCGAGGCGCGGCGAGCCGCAGCCATTGCC
TTTTATGGTAATCGTGCGAGAGGGCGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCAGGAAGGAAATGGGGGGGGAGGGCC
TTCGTGCGTCGCCGCGCCGCCGTCCCCTTCTCCCTCTCCAGCCTCGGGGCTGTCCGCGGGGGGACGGCTGCCTTC
GGGGGGGACGGGGCAGGGCGGGGTTCGGCTTCTGGCGTGTGACCGGCGGCTCTAGAGCCTCTGCTAACCATGTTC
ATGCCTTCTTCTTTTTCCTACAGCTCCTGGGCAACGTGCTGGTTATTGTGCTGTCTCATCATTTTGGCAAAGAAT
TCGATTGCCATGGCAACATATATCCAGAGAGTACAGTGCATCTCAACATCACTACTGGTTGTTCTCACCACATTG
GTCTCGTGTCAGATTCCCAGGGATAGGCTCTCTAACATAGGGGTCATAGTCGATGAAGGGAAATCACTGAAGATA
GCTGGATCCCACGAATCGAGGTACATAGTACTGAGTCTAGTTCCGGGGGTAGACTTTGAGAATGGGTGCGGAACA
GCCCAGGTTATCCAGTACAAGAGCCTACTGAACAGGCTGTTAATCCCATTGAGGGATGCCTTAGATCTTCAGGAG
GCTCTGATAACTGTCACCAATGATACGACACAAAATGCCGGTGCTCCCCAGTCGAGATTCTTCGGTGCTGTGATT
GGTACTATCGCACTTGGAGTGGCGACATCAGCACAAATCACCGCAGGGATTGCACTAGCCGAAGCGAGGGAGGCC
AAAAGAGACATAGCGCTCATCAAAGAATCGATGACAAAAACACACAAGTCTATAGAACTGCTGCAAAACGCTGTG
GGGGAACAAATTCTTGCTCTAAAGACACTCCAGGATTTCGTGAATGATGAGATCAAACCCGCAATAAGCGAATTA
GGCTGTGAGACTGCTGCCTTAAGACTGGGTATAAAATTGACACAGCATTACTCCGAGCTGTTAACTGCGTTCGGC
TCGAATTTCGGAACCATCGGAGAGAAGAGCCTCACGCTGCAGGCGCTGTCTTCACTTTACTCTGCTAACATTACT
GAGATTATGACCACAATCAGGACAGGGCAGTCTAACATCTATGATGTCATTTATACAGAACAGATCAAAGGAACG
GTGATAGATGTGGATCTAGAGAGATACATGGTCACCCTGTCTGTGAAGATCCCTATTCTTTCTGAAGTCCCAGGT
GTGCTCATACACAAGGCATCATCTATTTCTTACAACATAGACGGGGAGGAATGGTATGTGACTGTCCCCAGCCAT
ATACTCAGTCGTGCTTCTTTCTTAGGGGGTGCAGACATAACCGATTGTGTTGAGTCCAGATTGACCTATATATGC
CCCAGGGATCCCGCACAACTGATACCTGACAGCCAGCAAAAGTGTATCCTGGGGGACACAACAAGGTGTCCTGTC
ACAAAAGTTGTGGACAGCCTTATCCCCAAGTTTGCTTTTGTGAATGGGGGCGTTGTTGCTAACTGCATAGCATCC
ACATGTACCTGCGGGACAGGCCGAAGACCAATCAGTCAGGATCGCTCTAAAGGTGTAGTATTCCTAACCCATGAC
AACTGTGGTCTTATAGGTGTCAATGGGGTAGAATTGTATGCTAACCGGAGAGGGCACGATGCCACTTGGGGGGTC
CAGAACTTGACAGTCGGTCCTGCAATTGCTATCAGACCCGTTGATATTTCTCTCAACCTTGCTGATGCTACGAAT
TTCTTGCAAGACTCTAAGGCTGAGCTTGAGAAAGCACGGAAAATCCTCTCGGAGGTAGGTAGATGGTACAACTCA
AGAGAGACTGTGATTACGATCATAGTAGTTATGGTCGTAATATTGGTGGTCATTATAGTGATCATCATCGTGCTT
TATAGACTCAGAAGGTGAAATCACTAGTGAATTCACTCCTCAGGTGCAGGCTGCCTATCAGAAGGTGGTGGCTGG
TGTGGCCAATGCCCTGGCTCACAAATACCACTGAGATCTTTTTCCCTCTGCCAAAAATTATGGGGACATCATGAA
GCCCCTTGAGCATCTGACTTCTGGCTAATAAAGGAAATTTATTTTCATTGCAATAGTGTGTTGGAATTTTTTGTG
TCTCTCACTCGGAAGGACATATGGGAGGGCAAATCATTTAAAACATCAGAATGAGTATTTGGTTTAGAGTTTGGC
AACATATGCCCATATGCTGGCTGCCATGAACAAAGGTTGGCTATAAAGAGGTCATCAGTATATGAAACAGCCCCC
TGCTGTCCATTCCTTATTCCATAGAAAAGCCTTGACTTGAGGTTAGATTTTTTTTATATTTTGTTTTGTGTTATT
TTTTTCTTTAACATCCCTAAAATTTTCCTTACATGTTTTACTAGCCAGATTTTTCCTCCTCTCCTGACTACTCCC
AGTCATAGCTGTCCCTCTTCTCTTATGGAGATCCCTCGACCTGCAGCCCAAGCTTGGCGTAATCATGGTCATAGC
TGTTTCCTGTGTGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTCGGGAAACCTGT
CGTGCCAGCGGATCCGCATCTCAATTAGTCAGCAACCATAGTCCCGCCCCTAACTCCGCCCATCCCGCCCCTAAC
TCCGCCCAGTTCCGCCCATTCTCCGCCCCATGGCTGACTAATTTTTTTTATTTATGCAGAGGCCGAGGCCGCCTC
GGCCTCTGAGCTATTCCAGAAGTAGTGAGGAGGCTTTTTTGGAGGCCTAGGCTTTTGCAAAAAGCTAACTTGTTT
ATTGCAGCTTATAATGGTTACAAATAAAGCAATAGCATCACAAATTTCACAAATAAAGCATTTTTTTCACTGCAT
TCTAGTTGTGGTTTGTCCAAACTCATCAATGTATCTTATCATGTCTGTCCGCTTCCTCGCTCACTGACTCGCTGC
GCTCGGTCGTTCGGCTGCGGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAAAAGGCCGCGTTGCTGGCGT
TTTTCCATAGGCTCCGCCCCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGCCTTATCCGGTA
ACTATCGTCTTGAGTCCAACCCGGTAAGACACGACTTATCGCCACTGGCAGCAGCCACTGGTAACAGGATTAGCA
GAGCGAGGTATGTAGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGATTATCAA
AAAGGATCTTCACCTAGATCCTTTTAAATTAAAAATGAAGTTTTAAATCAATCTAAAGTATATATGAGTAAACTT
GGTCTGACAGTTAGAAAAACTCATCGAGCATCAAATGAAACTGCAATTTATTCATATCAGGATTATCAATACCAT
ATTTTTGAAAAAGCCGTTTCTGTAATGAAGGAGAAAACTCACCGAGGCAGTTCCATAGGATGGCAAGATCCTGGT
ATCGGTCTGCGATTCCGACTCGTCCAACATCAATACAACCTATTAATTTCCCCTCGTCAAAAATAAGGTTATCAA
GTGAGAAATCACCATGAGTGACGACTGAATCCGGTGAGAATGGCAACAGCTTATGCATTTCTTTCCAGACTTGTT
CAACAGGCCAGCCATTACGCTCGTCATCAAAATCACTCGCATCAACCAAACCGTTATTCATTCGTGATTGCGCCT
GAGCGAGACGAAATACGCGATCGCTGTTAAAAGGACAATTACAAACAGGAATCGAATGCAACCGGCGCAGGAACA
CTGCCAGCGCATCAACAATATTTTCACCTGAATCAGGATATTCTTCTAATACCTGGAATGCTGTTTTTCCGGGGA
TCGCAGTGGTGAGTAACCATGCATCATCAGGAGTACGGATAAAATGCTTGATGGTCGGAAGAGGCATAAATTCCG
TCAGCCAGTTTAGTCTGACCATCTCATCTGTAACATCATTGGCAACGCTACCTTTGCCATGTTTCAGAAACAACT
CTGGCGCATCGGGCTTCCCATACAATCGATAGATTGTCGCACCTGATTGCCCGACATTATCGCGAGCCCATTTAT
ACCCATATAAATCAGCATCCATGTTGGAATTTAATCGCGGCCTAGAGCAAGACGTTTCCCGTTGAATATGGCTCA
TAACACCCCTTGTATTACTGTTTATGTAAGCAGACAGTTTTATTGTTCATGATGATATATTTTTATCTTGTGCAA
TGTAACATCAGAGATTTTGAGACACAACAATTGGTCGAC
SEQ ID NO: 13 Plasmid as defined in FIG. 1G (pDNA3b pGM303)
ATTGATTATTGACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGGGCGCGCGCCAGGCGGGGCGGGGGGGG
GCGAGGGGCGGGGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCGCGTTACTCCCACAGG
TGAGCGGGCGGGACGGCCCTTCTCCTCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGGAGCGGCTCGGGGGGTGCGTGCGTGT
GTGTGTGCGTGGGGAGCGCCGCGTGCGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGTGCGGGGCTCCGTACGGGGCGTGGCG
CGGGGCTCGCCGTGCCGGGCGGGGGGGGGCGGCAGGTGGGGGTGCCGGGGGGGGCGGGGCCGCCTCGGGCCGGGG
AGGGCTCGGGGGAGGGGCGCGGCGGCCCCCGGAGCGCCGGCGGCTGTCGAGGCGCGGCGAGCCGCAGCCATTGCC
TTTTATGGTAATCGTGCGAGAGGGCGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCAGGAAGGAAATGGGGGGGGAGGGCC
TTCGTGCGTCGCCGCGCCGCCGTCCCCTTCTCCCTCTCCAGCCTCGGGGCTGTCCGCGGGGGGACGGGGCAGGGC
GGGGTTCGGCTTCTGGCGTGTGACCGGCGGCTCTAGAGCCTCTGCTAACCATGTTCATGCCTTCTTCTTTTTCCT
ACAGCTCCTGGGCAACGTGCTGGTTATTGTGCTGTCTCATCATTTTGGCAAAGAATTCCTCGAGCATGTGGTCTG
AGTTAAAAATCAGGAGCAACGACGGAGGTGAAGGACCAGAGGACGCCAACGACCCCCGGGGAAAGGGGGTGCAAC
ACATCCATATCCAGCCATCTCTACCTGTTTATGGACAGAGGGTTAGGGATGGTGATAGGGGCAAACGTGACTCGT
ACTGGTCTACTTCTCCTAGTGGTAGCACCACAAAACCAGCATCAGGTTGGGAGAGGTCAAGTAAAGCCGACACAT
GGTTGCTGATTCTCTCATTCACCCAGTGGGCTTTGTCAATTGCCACAGTGATCATCTGTATCATAATTTCTGCTA
GACAAGGGTATAGTATGAAAGAGTACTCAATGACTGTAGAGGCATTGAACATGAGCAGCAGGGAGGTGAAAGAGT
CACTTACCAGTCTAATAAGGCAAGAGGTTATAGCAAGGGCTGTCAACATTCAGAGCTCTGTGCAAACCGGAATCC
CAGTCTTGTTGAACAAAAACAGCAGGGATGTCATCCAGATGATTGATAAGTCGTGCAGCAGACAAGAGCTCACTC
AGCACTGTGAGAGTACGATCGCAGTCCACCATGCCGATGGAATTGCCCCACTTGAGCCACATAGTTTCTGGAGAT
GCCCTGTCGGAGAACCGTATCTTAGCTCAGATCCTGAAATCTCATTGCTGCCTGGTCCGAGCTTGTTATCTGGTT
CTACAACGATCTCTGGATGTGTTAGGCTCCCTTCACTCTCAATTGGCGAGGCAATCTATGCCTATTCATCAAATC
TCATTACACAAGGTTGTGCTGACATAGGGAAATCATATCAGGTCCTGCAGCTAGGGTACATATCACTCAATTCAG
ATATGTTCCCTGATCTTAACCCCGTAGTGTCCCACACTTATGACATCAACGACAATCGGAAATCATGCTCTGTGG
TGGCAACCGGGACTAGGGGTTATCAGCTTTGCTCCATGCCGACTGTAGACGAAAGAACCGACTACTCTAGTGATG
GTATTGAGGATCTGGTCCTTGATGTCCTGGATCTCAAAGGGAGAACTAAGTCTCACCGGTATCGCAACAGCGAGG
TAGATCTTGATCACCCGTTCTCTGCACTATACCCCAGTGTAGGCAACGGCATTGCAACAGAAGGCTCATTGATAT
TTCTTGGGTATGGTGGACTAACCACCCCTCTGCAGGGTGATACAAAATGTAGGACCCAAGGATGCCAACAGGTGT
CGCAAGACACATGCAATGAGGCTCTGAAAATTACATGGCTAGGAGGGAAACAGGTGGTCAGCGTGATCATCCAGG
TCAATGACTATCTCTCAGAGAGGCCAAAGATAAGAGTCACAACCATTCCAATCACTCAAAACTATCTCGGGGCGG
AAGGTAGATTATTAAAATTGGGTGATCGGGTGTACATCTATACAAGATCATCAGGCTGGCACTCTCAACTGCAGA
TAGGAGTACTTGATGTCAGCCACCCTTTGACTATCAACTGGACACCTCATGAAGCCTTGTCTAGACCAGGAAATA
AAGAGTGCAATTGGTACAATAAGTGTCCGAAGGAATGCATATCAGGCGTATACACTGATGCTTATCCATTGTCCC
CTGATGCAGCTAACGTCGCTACCGTCACGCTATATGCCAATACATCGCGTGTCAACCCAACAATCATGTATTCTA
ACACTACTAACATTATAAATATGTTAAGGATAAAGGATGTTCAATTAGAGGCTGCATATACCACGACATCGTGTA
TCACGCATTTTGGTAAAGGCTACTGCTTTCACATCATCGAGATCAATCAGAAGAGCCTGAATACCTTACAGCCGA
TGCTCTTTAAGACTAGCATCCCTAAATTATGCAAGGCCGAGTCTTAAGCGGCCGCGCATGCGAATTCACTCCTCA
GGTGCAGGCTGCCTATCAGAAGGTGGTGGCTGGTGTGGCCAATGCCCTGGCTCACAAATACCACTGAGATCTTTT
TCCCTCTGCCAAAAATTATGGGGACATCATGAAGCCCCTTGAGCATCTGACTTCTGGCTAATAAAGGAAATTTAT
TTTCATTGCAATAGTGTGTTGGAATTTTTTGTGTCTCTCACTCGGAAGGACATATGGGAGGGCAAATCATTTAAA
ACATCAGAATGAGTATTTGGTTTAGAGTTTGGCAACATATGCCCATATGCTGGCTGCCATGAACAAAGGTTGGCT
ATAAAGAGGTCATCAGTATATGAAACAGCCCCCTGCTGTCTATTCCTTATTCCATAGAAAAGCCTTGACTTGAGG
TTAGATTTTTTTTATATTTTGTTTTGTGTTATTTTTTTCTTTAACATCCCTAAAATTTTCCTTACATGTTTTACT
AGCCAGATTTTTCCTCCTCTCCTGACTACTCCCAGTCATAGCTGTCCCTCTTCTCTTATGGAGATCCCTCGACCT
GCAGCCCAAGCTTGGCGTAATCATGGTCATAGCTGTTTCCTGTGTGAAATTGTTATCCGCTCACAATTCCACACA
ACATACGAGCCGGAAGCATAAAGTGTAAAGCCTGGGGTGCCTAATGAGTGAGCTAACTCACATTAATTGCGTTGC
GCTCACTGCCCGCTTTCCAGTCGGGAAACCTGTCGTGCCAGCGGATCCGCATCTCAATTAGTCAGCAACCATAGT
CCCGCCCCTAACTCCGCCCATCCCGCCCCTAACTCCGCCCAGTTCCGCCCATTCTCCGCCCCATGGCTGACTAAT
TTTTTTTATTTATGCAGAGGCCGAGGCCGCCTCGGCCTCTGAGCTATTCCAGAAGTAGTGAGGAGGCTTTTTTGG
AGGCCTAGGCTTTTGCAAAAAGCTAACTTGTTTATTGCAGCTTATAATGGTTACAAATAAAGCAATAGCATCACA
AATTTCACAAATAAAGCATTTTTTTCACTGCATTCTAGTTGTGGTTTGTCCAAACTCATCAATGTATCTTATCAT
GTCTGTCCGCTTCCTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAAGGCCAGCAAAAGG
CCAGGAACCGTAAAAAGGCCGCGTTGCTGGCGTTTTTCCATAGGCTCCGCCCCCCTGACGAGCATCACAAAAATC
GACGCTCAAGTCAGAGGTGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCCAACCCGGTAAGACACGACTTATCGC
CACTGGCAGCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTTAAATTAAAAATGAAGTT
TTAAATCAATCTAAAGTATATATGAGTAAACTTGGTCTGACAGTTAGAAAAACTCATCGAGCATCAAATGAAACT
GCAATTTATTCATATCAGGATTATCAATACCATATTTTTGAAAAAGCCGTTTCTGTAATGAAGGAGAAAACTCAC
CGAGGCAGTTCCATAGGATGGCAAGATCCTGGTATCGGTCTGCGATTCCGACTCGTCCAACATCAATACAACCTA
TTAATTTCCCCTCGTCAAAAATAAGGTTATCAAGTGAGAAATCACCATGAGTGACGACTGAATCCGGTGAGAATG
GCAACAGCTTATGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAAAAGGACAATTAC
AAACAGGAATCGAATGCAACCGGCGCAGGAACACTGCCAGCGCATCAACAATATTTTCACCTGAATCAGGATATT
CTTCTAATACCTGGAATGCTGTTTTTCCGGGGATCGCAGTGGTGAGTAACCATGCATCATCAGGAGTACGGATAA
AATGCTTGATGGTCGGAAGAGGCATAAATTCCGTCAGCCAGTTTAGTCTGACCATCTCATCTGTAACATCATTGG
CAACGCTACCTTTGCCATGTTTCAGAAACAACTCTGGCGCATCGGGCTTCCCATACAATCGATAGATTGTCGCAC
CTGATTGCCCGACATTATCGCGAGCCCATTTATACCCATATAAATCAGCATCCATGTTGGAATTTAATCGCGGCC
TAGAGCAAGACGTTTCCCGTTGAATATGGCTCATAACACCCCTTGTATTACTGTTTATGTAAGCAGACAGTTTTA
TTGTTCATGATGATATATTTTTATCTTGTGCAATGTAACATCAGAGATTTTGAGACACAACAATTGGTCGAC
SEQ ID NO: 14 Exemplified WPRE component (mWPRE)

| | |
|---|---|
| 1 | GGGCCCAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCCGCAAGCT |

SEQ ID NO: 15 Exemplary CAG promoter
ATTGATTATTGACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGGGCGCGCGCCAGGCGGGGCGGGGCGGG
GCGAGGGGCGGGGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCGCGTTACTCCCACAGG
TGAGCGGGCGGGACGGCCCTTCTCCTCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGGAGCGGCTCGGGGGGTGCGTGCGTGT
GTGTGTGCGTGGGGAGCGCCGCGTGCGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGTGCGGGGCTCCGTACGGGGCGTGGCG
CGGGGCTCGCCGTGCCGGGCGGGGGGTGGCGGCAGGTGGGGGTGCCGGGCGGGGCGGGGCCGCCTCGGGCCGGGG
AGGGCTCGGGGGAGGGGCGCGGCGGCCCCCGGAGCGCCGGCGGCTGTCGAGGCGCGGCGAGCCGCAGCCATTGCC
TTTTATGGTAATCGTGCGAGAGGGCGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCAGGAAGGAAATGGGGGGGGAGGGCC
TTCGTGCGTCGCCGCGCCGCCGTCCCCTTCTCCCTCTCCAGCCTCGGGGCTGTCCGCGGGGGGACGGCTGCCTTC
GGGGGGGACGGGGCAGGGCGGGGTTCGGCTTCTGGCGTGTGACCGGCGGCTCTAGAGCCTCTGCTAACCATGTTC
ATGCCTTCTTCTTTTTCCTACAGCTCCTGGGCAACGTGCTGGTTATTGTGCTGTCTCATCATTTTGGCAAAGAAT
TGCTCGAGCCACC
SEQ ID NO: 16 Modified SIV/CFTR RNA sequence

| | |
|---|---|
| ucucuuacua ggagaccagc uugagccugg guguucgcug guuagccuaa ccugguuggc | 60 |
| caccaggggu aaggacuccu uggcuuagaa agcuaauaaa cuugccugca uuagagcuua | 120 |
| ucugagucaa guguccucau ugacgccuca cucucuugaa cgggaaucuu ccuuacuggg | 180 |
| uucucucucu gacccaggcg agagaaacuc cagcaguggc gcccgaacag ggacuugagu | 240 |
| gagaguguag gcacguacag cugagaaggc gucggacgcg aaggaagcgc ggggugcgac | 300 |
| gcgaccaaga aggagacuug gugaguaggc uucucgagug ccgggaaaaa gcucgagccu | 360 |
| aguuagagga cuaggagagg ccguagccgu aacuacucug ggcaaguagg gcaggcggug | 420 |
| gguacgcaau ugggggcggc uaccucagca cuaaauagga gacaauuaga ccaauuugag | 480 |
| aaaauacgac uucgcccgaa cggaaagaaa aaguaccaaa uuaaacauuu aauauugggc | 540 |
| aggcaaggag auuggagcgc uucggccucc augagagguu guuggagaca gaggaggggu | 600 |
| guaaaagaau cauagaaguc cucuaccccc uagaaccaac aggaucggag ggcuuaaaaa | 660 |
| gucuguucaa ucuugugugc gugcuauauu gcuugcacaa ggaacagaaa gugaaagaca | 720 |
| cagaggaagc aguagcaaca guaagacaac acugccaucu aguggaaaaa gaaaaaagug | 780 |
| caacagagac aucuagugga caaaagaaaa augacaaggg aauagcagcg ccaccuggug | 840 |
| gcagucagaa uuuuccagcg caacaacaag gaaauugccu ggguacaugu acccuuguca | 900 |
| ccgcgcaccu uaaaugcgug gguaaaagca guagaggaga aaaaauuugg agcagaaaua | 960 |
| guacccaugu uucaagcccu aucgccugca ggccguuugu gcuaggguuc uuaggcuucu | 1020 |
| ugggggcugc uggaacugca uugggagcag cggcgacagc ccugacgguc cagucucagc | 1080 |
| auuugcuugc ugggauacug cagcagcaga agaaucugcu ggcggcugug gaggcucaac | 1140 |
| agcagauguu gaagcugacc auuuggggug uuaaaaaccu caaugcccgc gucacagccc | 1200 |
| uugagaagua ccuagaggau caggcacgac uaaacuccug ggggugcgca uggaaacaag | 1260 |
| uaugucauac cacaguggag uggcccugga caaaucggac uccggauugg caaaauaaga | 1320 |
| cuugguugga gugggaaaga caaauagcug auuuggaaag caacauuacg agacaauuag | 1380 |
| ugaaggcuag agaacaagag gaaaagaauc uagaugccua ucagaaguua acuaguuggu | 1440 |
| cagauuucug gucuugguuc gauuucucaa aauggcuuaa cauuuuaaaa aagggauuuu | 1500 |
| uaguaauagu aggaauaaua ggguuaagau uacuuuacac aguauaugga uguauaguga | 1560 |
| ggguuaggca gggauauguu ccucuaucuc cacagaucca uauaaagcgg caauuuuaaa | 1620 |
| agaaagggag gaauaggggg acagacuuca gcagagagac uaauuaauau aauaacaaca | 1680 |
| caauuagaaa uacaacauuu acaaaccaaa auucaaaaaa uuuuaaauuu uagagccgcg | 1740 |
| gagaucuguu acauaacuua ugguaaaugg ccugccuggc ugacugccca augaccccug | 1800 |
| cccaaugaug ucaauaauga uguauguucc cauguaaugc caauagggac uuuccauuga | 1860 |
| ugucaauggg uggaguauuu augguaacug cccacuuggc aguacaucaa guguaucaua | 1920 |
| ugccaaguau gcccccuauu gaugucaaug augguaaaug gccugccugg cauuaugccc | 1980 |
| aguacaugac cuuaugggac uuuccuacuu ggcaguacau cuauguauua gucauugcua | 2040 |
| uuaccauggg aauucacuag uggagaagag caugcuugag ggcugagugc cccucagugg | 2100 |
| gcagagagca cauggcccac agucccugag aaguuggggg gagggguggg caauugaacu | 2160 |
| ggugccuaga gaaggugggg cuuggguaaa cugggaaagu gauguggugu acuggcucca | 2220 |
| ccuuuuuccc cagggugggg gagaaccaua uauaagugca guagucucug ugaacauuca | 2280 |
| agcuucugcc uucucccucc ugugaguuug cuagccacca ugcagagaag cccucuggag | 2340 |
| aaggccucug uggugagcaa gcuguucuuc agcuggacca ggcccauccu gaggaagggc | 2400 |
| uacaggcaga gacuggagcu gucugacauc uaccagaucc ccucugugga cucugcugac | 2460 |
| aaccugucug agaagcugga gagggagugg gauagagagc uggccagcaa gaagaacccc | 2520 |
| aagcugauca augcccugag gagaugcuuc uucuggagau ucauguucua uggcaucuuc | 2580 |
| cuguaccugg gggaagugac caaggcugug cagccucugc ugcugggcag aaucauugcc | 2640 |
| agcuaugacc cugacaacaa ggaggagagg agcauugcca ucuaccuggg cauuggccug | 2700 |
| ugccugcugu ucauugugag gacccugcug cugcacccug ccaucuuugg ccugcaccac | 2760 |
| auuggcaugc agaugaggau ugccauguuc agccugaucu acaagaaaac ccugaagcug | 2820 |
| uccagcagag ugcuggacaa gaucagcauu ggccagcugg ugagccugcu gagcaacaac | 2880 |
| cugaacaagu uugaugaggg ccuggcccug gcccacuuug uguggauugc cccucugcag | 2940 |
| guggcccugc ugaugggccu gauuugggag cugcugcagg ccucugccuu uuguggccug | 3000 |
| ggcuuccuga uugugcuggc ccuguuucag gcuggccugg gcaggaugau gaugaaguac | 3060 |
| agggaccaga gggcaggcaa gaucagugag aggcugguga ucaccucuga gaugauugag | 3120 |
| aacauccagu cugugaaggc cuacuguugg gaggaagcua uggagaagau gauugaaaac | 3180 |
| cugaggcaga cagagcugaa gcugaccagg aaggcugccu augugagaua cuucaacagc | 3240 |
| ucugccuucu ucuucucugg cuucuuugug guguuccugu cugugcugcc cuaugcccug | 3300 |
| aucaagggga ucauccugag aaagauuuuc accaccauca gcuucugcau ugugcugagg | 3360 |
| auggcuguga ccagacaguu ccccugggcu gugcagaccu gguaugacag ccugggggcc | 3420 |
| aucaacaaga uccaggacuu ccugcagaag caggaguaca agacccugga guacaaccug | 3480 |
| accaccacag aaguggugau ggagaaugug acagccuucu gggaggaggg cuuuggggag | 3540 |
| cuguuugaga aggccaagca gaacaacaac aacagaaaga ccagcaaugg ggaugacucc | 3600 |
| cuguucuucu ccaacuucuc ccugcugggc acaccugugc ugaaggacau caacuucaag | 3660 |
| auugagaggg ggcagcugcu ggcuguggcu ggaucuacag gggcuggcaa gaccagccug | 3720 |
| cugaugauga ucauggggga gcuggagccu ucugagggca agaucaagca cucuggcagg | 3780 |
| aucagcuuuu gcagccaguu cagcuggauc augccuggca ccaucaagga gaacaucauc | 3840 |
| uuuggaguga gcuaugauga guacagauac aggaguguga ucaaggccug ccagcuggag | 3900 |
| gaggacauca gcaaguuugc ugagaaggac aacauugugc ugggggaggg aggcauuaca | 3960 |
| cugucugggg gccagagagc cagaaucagc cuggccaggg cuguguacaa ggaugcugac | 4020 |
| cuguaccugc uggacucccc cuuuggcuac cuggaugugc ugacagagaa ggagauuuuu | 4080 |
| gagagcugug ugugcaagcu gauggccaac aagaccagaa uccuggugac cagcaagaug | 4140 |
| gagcaccuga agaaggcuga caagauccug auccugcaug agggcagcag cuacuucuau | 4200 |
| gggaccuucu cugagcugca gaaccugcag ccugacuuca gcucuaagcu gaugggcugu | 4260 |
| gacagcuuug accaguucuc ugcugagagg aggaacagca uccugacaga gacccugcac | 4320 |
| agauucagcc uggagggaga ugccccugug agcuggacag agaccaagaa gcagagcuuc | 4380 |
| aagcagacag gggaguuugg ggagaagagg aagaacucca uccugaaccc caucaacagc | 4440 |
| aucaggaagu ucagcauugu gcagaaaacc ccccugcaga ugaauggcau ugaggaagau | 4500 |
| ucugaugagc cccuggagag gagacugagc cuggugccug auucugagca gggagaggcc | 4560 |
| auccugccua ggaucucugu gaucagcaca ggcccuacac ugcaggccag aaggaggcag | 4620 |
| ucugugcuga accugaugac ccacucugug aaccagggcc agaacaucca caggaaaacc | 4680 |
| acagccucca ccaggaaagu gagccuggcc ccucaggcca aucugacaga gcuggacauc | 4740 |
| uacagcagga ggcugucuca ggagacaggc cuggagauuu cugaggagau caaugaggag | 4800 |
| gaccugaaag agugcuucuu ugaugacaug gagagcaucc cugcugugac caccuggaac | 4860 |
| accuaccuga gauacaucac agugcacaag agccugaucu uugugcugau cuggugccug | 4920 |
| gugaucuucc uggcugaagu ggcugccucu cugguggugc uguggcugcu gggaaacacc | 4980 |
| ccacugcagg acaagggcaa cagcacccac agcaggaaca acagcuaugc ugugaucauc | 5040 |
| accuccaccu ccagcuacua uguguucuac aucuaugugg gaguggcuga uacccugcug | 5100 |
| gcuaugggcu ucuuuagagg ccugccccug gugcacacac ugaucacagu gagcaagauc | 5160 |
| cuccaccaca agaugcugca cucugugcug caggcuccua ugagcacccu gaauacccug | 5220 |
| aaggcugggg gcauccugaa cagauucucc aaggauauug ccauccugga ugaccugcug | 5280 |
| ccucucacca ucuuugacuu cauccagcug cugcugauug ugauuggggc cauugcugug | 5340 |
| guggcagugc ugcagcccua caucuuugug gccacagugc cugugauugu ggccuucauc | 5400 |
| augcugaggg ccuacuuucu gcagaccucc cagcagcuga agcagcugga gucugagggc | 5460 |
| agaagcccca ucuucaccca ccuggugaca agccugaagg gccuguggac ccugagagcc | 5520 |
| uuuggcaggc agcccuacuu ugagacccug uuccacaagg cccugaaccu gcacacagcc | 5580 |
| aacugguucc ucuaccuguc cacccugaga ugguuccaga ugagaauuga gaugaucuuu | 5640 |
| gucaucuucu ucauugcugu gaccuucauc agcauucuga ccacaggaga gggagagggc | 5700 |
| agagugggca uuauccugac ccuggccaug aacaucauga gcacacugca gugggcagug | 5760 |
| aacagcagca uugaugugga cagccugaug aggaguguga gcagaguguu caaguucauu | 5820 |
| gauaugccca cagagggcaa gccuaccaag agcaccaagc ccuacaagaa uggccagcug | 5880 |
| agcaaaguga ugaucauuga gaacagccau gugaagaagg augauaucug gcccagugga | 5940 |
| ggccagauga cagugaagga ccugacagcc aaguacacag aggggggcaa ugcuauccug | 6000 |
| gagaacaucu ccuucagcau cuccccuggc cagagagugg gacugcuggg aagaacaggc | 6060 |
| ucuggcaagu cuacccugcu gucugccuuc cugaggcugc ugaacacaga gggagagauc | 6120 |
| cagauugaug gaguguccug ggacagcauc acacugcagc aguggaggaa ggccuuuggu | 6180 |
| gugauccccc agaaaguguu caucuucagu ggcaccuuca ggaagaaccu ggaccccuau | 6240 |
| gagcaguggu cugaccagga gauuuggaaa guggcugaug aagugggccu gagaagugug | 6300 |
| auugagcagu ucccuggcaa gcuggacuuu guccuggugg augggggcug ugugcugagc | 6360 |
| cauggccaca agcagcugau gugccuggcc agaucagugc ugagcaaggc caagauccug | 6420 |
| cugcuggaug agccuucugc ccaccuggau ccugugaccu accagaucau caggaggacc | 6480 |
| cucaagcagg ccuuugcuga cugcacaguc auccugugug agcacaggau ugaggccaug | 6540 |
| cuggagugcc agcaguuccu ggugauugag gagaacaaag ugaggcagua ugacagcauc | 6600 |
| cagaagcugc ugaaugagag gagccuguuc aggcaggcca ucagccccuc ugauagagug | 6660 |
| aagcuguucc cccacaggaa cagcuccaag ugcaagagca agccccagau ugcugcccug | 6720 |
| aaggaggaga cagaggagga agugcaggac accaggcugu gagggcccaa ucaaccucug | 6780 |
| gauuacaaaa uuugugaaag auugacuggu auucuuaacu auguugcucc uuuuacgcua | 6840 |
| uguggauacg cugcuuuaau gccuuuguau caugcuauug cuucccguau ggcuuucauu | 6900 |
| uucuccuccu uguauaaauc cugguugcug ucucuuuaug aggaguugug gcccguuguc | 6960 |
| aggcaacgug gcguggugug cacuguguuu gcugacgcaa cccccacugg uuggggcauu | 7020 |
| gccaccaccu gucagcuccu uuccgggacu uucgcuuucc cccucccuau ugccacggcg | 7080 |
| gaacucaucg ccgccugccu ugcccgcugc uggacagggg cucggcuguu gggcacugac | 7140 |
| aauuccgugg uguugucggg gaaaucaucg uccuuuccuu ggcugcucgc cuguguugcc | 7200 |
| accuggauuc ugcgcgggac guccuucugc uacgucccuu cggcccucaa uccagcggac | 7260 |
| cuuccuuccc gcggccugcu gccggcucug cggccucuuc cgcgucuucg ccuucgcccu | 7320 |
| cagacgaguc ggaucucccu uugggccgcc uccccgcaag cuucgcacuu uuuaaaagaa | 7380 |
| aagggaggac uggaugggau uuauuacucc gauaggacgc uggcuuguaa cucagucucu | 7440 |
| uacuaggaga ccagcuugag ccuggguguu cgcugguuag ccuaaccugg uuggccacca | 7500 |
| gggguaagga cuccuuggcu uagaaagcua auaaacuugc cugcauuaga gcu | 7553 |

```
SEQ ID NO: 17 Fct4 protein
Gln Ile Pro Arg Asp Arg Leu Ser Asn Ile Gly Val Ile Val Asp Glu
1 5 10 15
Gly Lys Ser Leu Lys Ile Ala Gly Ser His Glu Ser Arg Tyr Ile Val
                        20 25 30
Leu Ser Leu Val Pro Gly Val Asp Phe Glu Asn Gly Cys Gly Thr Ala
                35 40 45
Gln Val Ile Gln Tyr Lys Ser Leu Leu Asn Arg Leu Leu Ile Pro Leu
        50 55 60
Arg Asp Ala Leu Asp Leu Gln Glu Ala Leu Ile Thr Val Thr Asn Asp
65 70 75 80
Thr Thr Gln Asn Ala Gly Ala Pro Gln Ser Arg Phe Phe Gly Ala Val
                                85 90 95
Ile Gly Thr Ile Ala Leu Gly Val Ala Thr Ser Ala Gln Ile Thr Ala
                        100 105 110
Gly Ile Ala Leu Ala Glu Ala Arg Glu Ala Lys Arg Asp Ile Ala Leu
                115 120 125
Ile Lys Glu Ser Met Thr Lys Thr His Lys Ser Ile Glu Leu Leu Gln
        130 135 140
Asn Ala Val Gly Glu Gln Ile Leu Ala Leu Lys Thr Leu Gln Asp Phe
145 150 155 160
Val Asn Asp Glu Ile Lys Pro Ala Ile Ser Glu Leu Gly Cys Glu Thr
                                165 170 175
Ala Ala Leu Arg Leu Gly Ile Lys Leu Thr Gln His Tyr Ser Glu Leu
                        180 185 190
Leu Thr Ala Phe Gly Ser Asn Phe Gly Thr Ile Gly Glu Lys Ser Leu
                195 200 205
Thr Leu Gln Ala Leu Ser Ser Leu Tyr Ser Ala Asn Ile Thr Glu Ile
        210 215 220
Met Thr Thr Ile Arg Thr Gly Gln Ser Asn Ile Tyr Asp Val Ile Tyr
225 230 235 240
Thr Glu Gln Ile Lys Gly Thr Val Ile Asp Val Asp Leu Glu Arg Tyr
                                245 250 255
Met Val Thr Leu Ser Val Lys Ile Pro Ile Leu Ser Glu Val Pro Gly
                        260 265 270
Val Leu Ile His Lys Ala Ser Ser Ile Ser Tyr Asn Ile Asp Gly Glu
                275 280 285
Glu Trp Tyr Val Thr Val Pro Ser His Ile Leu Ser Arg Ala Ser Phe
        290 295 300
Leu Gly Gly Ala Asp Ile Thr Asp Cys Val Glu Ser Arg Leu Thr Tyr
305 310 315 320
Ile Cys Pro Arg Asp Pro Ala Gln Leu Ile Pro Asp Ser Gln Gln Lys
                                325 330 335
Cys Ile Leu Gly Asp Thr Thr Arg Cys Pro Val Thr Lys Val Val Asp
                        340 345 350
Ser Leu Ile Pro Lys Phe Ala Phe Val Asn Gly Gly Val Val Ala Asn
                355 360 365
Cys Ile Ala Ser Thr Cys Thr Cys Gly Thr Gly Arg Arg Pro Ile Ser
        370 375 380
Gln Asp Arg Ser Lys Gly Val Val Phe Leu Thr His Asp Asn Cys Gly
385 390 395 400
Leu Ile Gly Val Asn Gly Val Glu Leu Tyr Ala Asn Arg Arg Gly His
                                405 410 415
Asp Ala Thr Trp Gly Val Gln Asn Leu Thr Val Gly Pro Ala Ile Ala
                        420 425 430
Ile Arg Pro Val Asp Ile Ser Leu Asn Leu Ala Asp Ala Thr Asn Phe
                435 440 445
Leu Gln Asp Ser Lys Ala Glu Leu Glu Lys Ala Arg Lys Ile Leu Ser
        450 455 460
Glu Val Gly Arg Trp Tyr Asn Ser Arg Glu Thr Val Ile Thr Ile Ile
465 470 475 480
Val Val Met Val Val Ile Leu Val Val Ile Ile Val Ile Ile Ile Val
                                485 490 495
Leu Tyr Arg Leu Arg Arg
SEQ ID NO: 18 Fct4 protein (including signal sequence)
Met Ala Thr Tyr Ile Gln Arg Val Gln Cys Ile Ser Thr Ser Leu Leu 1 5 10 15
Val Val Leu Thr Thr Leu Val Ser Cys Gln Ile Pro Arg Asp Arg Leu
                        20 25 30
Ser Asn Ile Gly Val Ile Val Asp Glu Gly Lys Ser Leu Lys Ile Ala
                35 40 45
Gly Ser His Glu Ser Arg Tyr Ile Val Leu Ser Leu Val Pro Gly Val
        50 55 60
Asp Phe Glu Asn Gly Cys Gly Thr Ala Gln Val Ile Gln Tyr Lys Ser
65 70 75 80
Leu Leu Asn Arg Leu Leu Ile Pro Leu Arg Asp Ala Leu Asp Leu Gln
                                85 90 95
Glu Ala Leu Ile Thr Val Thr Asn Asp Thr Thr Gln Asn Ala Gly Ala
                        100 105 110
Pro Gln Ser Arg Phe Phe Gly Ala Val Ile Gly Thr Ile Ala Leu Gly
                115 120 125
Val 130 Thr Ser Ala Gln 135 Thr Ala Gly Ile 140 Leu Ala Glu Ala
        130 135 140
Arg Glu Ala Lys Arg Asp Ile Ala Leu Ile Lys Glu Ser Met Thr Lys
145 150 155 160
Thr His Lys Ser Ile Glu Leu Leu Gln Asn Ala Val Gly Glu Gln Ile
                                165 170 175
Leu Ala Leu Lys Thr Leu Gln Asp Phe Val Asn Asp Glu Ile Lys Pro
                        180 185 190
Ala Ile Ser Glu Leu Gly Cys Glu Thr Ala Ala Leu Arg Leu Gly Ile
                195 200 205
Lys Leu Thr Gln His Tyr Ser Glu Leu Leu Thr Ala Phe Gly Ser Asn
        210 215 220
Phe Gly Thr Ile Gly Glu Lys Ser Leu Thr Leu Gln Ala Leu Ser Ser
225 230 235 240
Leu Tyr Ser Ala Asn Ile Thr Glu Ile Met Thr Thr Ile Arg Thr Gly
                                245 250 255
Gln Ser Asn Ile Tyr Asp Val Ile Tyr Thr Glu Gln Ile Lys Gly Thr
                        260 265 270
Val Ile Asp Val Asp Leu Glu Arg Tyr Met Val Thr Leu Ser Val Lys
                275 280 285
Ile Pro Ile Leu Ser Glu Val Pro Gly Val Leu Ile His Lys Ala Ser
        290 295 300
Ser Ile Ser Tyr Asn Ile Asp Gly Glu Glu Trp Tyr Val Thr Val Pro
305 310 315 320
Ser His Ile Leu Ser Arg Ala Ser Phe Leu Gly Gly Ala Asp Ile Thr
                                325 330 335
Asp Cys Val Glu Ser Arg Leu Thr Tyr Ile Cys Pro Arg Asp Pro Ala
                        340 345 350
Gln Leu Ile Pro Asp Ser Gln Gln Lys Cys Ile Leu Gly Asp Thr Thr
                355 360 365
Arg Cys Pro Val Thr Lys Val Val Asp Ser Leu Ile Pro Lys Phe Ala
        370 375 380
Phe Val Asn Gly Gly Val Val Ala Asn Cys Ile Ala Ser Thr Cys Thr
385 390 395 400
Cys Gly Thr Gly Arg Arg Pro Ile Ser Gln Asp Arg Ser Lys Gly Val
                                405 410 415
Val Phe Leu Thr His Asp Asn Cys Gly Leu Ile Gly Val Asn Gly Val
                        420 425 430
Glu Leu Tyr Ala Asn Arg Arg Gly His Asp Ala Thr Trp Gly Val Gln
                435 440 445
Asn Leu Thr Val Gly Pro Ala Ile Ala Ile Arg Pro Val Asp Ile Ser
        450 455 460
Leu Asn Leu Ala Asp Ala Thr Asn Phe Leu Gln Asp Ser Lys Ala Glu
465 470 475 480
Leu Glu Lys Ala Arg Lys Ile Leu Ser Glu Val Gly Arg Trp Tyr Asn
                                485 490 495
Ser Arg Glu Thr Val Ile Thr Ile Ile Val Val Met Val Val Ile Leu
                        500 505 510
Val Val Ile Ile Val Ile Ile Ile Val Leu Tyr Arg Leu Arg Arg
                515 520 525
SEQ ID NO: 19 Fct4 protein (fragment 1)
Phe Phe Gly Ala Val Ile Gly Thr Ile Ala Leu Gly Val Ala Thr Ser
1 5 10 15
Ala Gln Ile Thr Ala Gly Ile Ala Leu Ala Glu Ala Arg Glu Ala Lys
                        20 25 30
Arg Asp Ile Ala Leu Ile Lys Glu Ser Met Thr Lys Thr His Lys Ser
                35 40 45
Ile Glu Leu Leu Gln Asn Ala Val Gly Glu Gln Ile Leu Ala Leu Lys
        50 55 60
Thr Leu Gln Asp Phe Val Asn Asp Glu Ile Lys Pro Ala Ile Ser Glu
65 70 75 80
Leu Gly Cys Glu Thr Ala Ala Leu Arg Leu Gly Ile Lys Leu Thr Gln
                                85 90 95
His Tyr Ser Glu Leu Leu Thr Ala Phe Gly Ser Asn Phe Gly Thr Ile
                        100 105 110
Gly Glu Lys Ser Leu Thr Leu Gln Ala Leu Ser Ser Leu Tyr Ser Ala
                115 120 125
Asn Ile Thr Glu Ile Met Thr Thr Ile Arg Thr Gly Gln Ser Asn Ile
        130 135 140
Tyr Asp Val Ile Tyr Thr Glu Gln Ile Lys Gly Thr Val Ile Asp Val
145 150 155 160
Asp Leu Glu Arg Tyr Met Val Thr Leu Ser Val Lys Ile Pro Ile Leu
                                165 170 175
Ser Glu Val Pro Gly Val Leu Ile His Lys Ala Ser Ser Ile Ser Tyr
                        180 185 190
Asn Ile Asp Gly Glu Glu Trp Tyr Val Thr Val Pro Ser His Ile Leu
                195 200 205
Ser Arg Ala Ser Phe Leu Gly Gly Ala Asp Ile Thr Asp Cys Val Glu
        210 215 220
Ser Arg Leu Thr Tyr Ile Cys Pro Arg Asp Pro Ala Gln Leu Ile Pro
225 230 235 240
Asp Ser Gln Gln Lys Cys Ile Leu Gly Asp Thr Thr Arg Cys Pro Val
                                245 250 255
Thr Lys Val Val Asp Ser Leu Ile Pro Lys Phe Ala Phe Val Asn Gly
                        260 265 270
Gly Val Val Ala Asn Cys Ile Ala Ser Thr Cys Thr Cys Gly Thr Gly
                275 280 285
Arg Arg Pro Ile Ser Gln Asp Arg Ser Lys Gly Val Val Phe Leu Thr
        290 295 300
His Asp Asn Cys Gly Leu Ile Gly Val Asn Gly Val Glu Leu Tyr Ala
305 310 315 320
Asn Arg Arg Gly His Asp Ala Thr Trp Gly Val Gln Asn Leu Thr Val
                                325 330 335
Gly Pro Ala Ile Ala Ile Arg Pro Val Asp Ile Ser Leu Asn Leu Ala
                        340 345 350
Asp Ala Thr Asn Phe Leu Gln Asp Ser Lys Ala Glu Leu Glu Lys Ala
                355 360 365
Arg Lys Ile Leu Ser Glu Val Gly Arg Trp Tyr Asn Ser Arg Glu Thr
        370 375 380
Val Ile Thr Ile Ile Val Val Met Val Val Ile Leu Val Val Ile Ile
385 390 395 400
Val Ile Ile Ile Val Leu Tyr Arg Leu Arg Arg
                                405 410
SEQ ID NO: 20 Fct4 protein (fragment 2)
Gln Ile Pro Arg Asp Arg Leu Ser Asn Ile Gly Val Ile Val Asp Glu
1 5 10 15
Gly Lys Ser Leu Lys Ile Ala Gly Ser His Glu Ser Arg Tyr Ile Val
                        20 25 30
Leu Ser Leu Val Pro Gly Val Asp Phe Glu Asn Gly Cys Gly Thr Ala
                35 40 45
Gln 50 Ile Gln Tyr Lys Ser Leu Leu Asn Arg Leu Leu Ile Pro Leu
        50 55 60
Arg Asp Ala Leu Asp Leu Gln Glu Ala Leu Ile Thr Val Thr Asn Asp
65 70 75 80
Thr Thr Gln Asn Ala Gly Ala Pro Gln Ser Arg
                                85 90
SEQ ID NO: 21 Fct4 protein signal sequence
```

| | |
|---|---|
| MATYIQRVOC ISTSLLVVLT TLVSC | 25 |

```
SEQ ID NO: 22 p17 protein sequence
Gly Ala Ala Thr Ser Ala Leu Asn Arg Arg Gln Leu Asp Gln Phe Glu
1 5 10 15
Lys Ile Arg Leu Arg Pro Asn Gly Lys Lys Lys Tyr Gln Ile Lys His
                        20 25 30
Leu Ile Trp Ala Gly Lys Glu Met Glu Arg Phe Gly Leu His Glu Arg
                35 40 45
Leu Leu Glu Thr Glu Glu Gly Cys Lys Arg Ile Ile Glu Val Leu Tyr
        50 55 60
Pro Leu Glu Pro Thr Gly Ser Glu Gly Leu Lys Ser Leu Phe Asn Leu
65 70 75 80
Val Cys Val Leu Tyr Cys Leu His Lys Glu Gln Lys Val Lys Asp Thr
                                85 90 95
Glu Glu Ala Val Ala Thr Val Arg Gln His Cys His Leu Val Glu Lys
                        100 105 110
Glu Lys Ser Ala Thr Glu Thr Ser Ser Gly Gln Lys Lys Asn Asp Lys
                115 120 125
Gly Ile Ala Ala Pro Pro Gly Gly Ser Gln Asn Phe
        130 135 140
SEQ ID NO: 23 p24 protein sequence
Pro Ala Gln Gln Gln Gly Asn Ala Trp Val His Val Pro Leu Ser Pro
1 5 10 15
Arg Thr Leu Asn Ala Trp Val Lys Ala Val Glu Glu Lys Lys Phe Gly
                        20 25 30
Ala Glu Ile Val Pro Met Phe Gln Ala Leu Ser Glu Gly Cys Thr Pro
                35 40 45
Tyr Asp Ile Asn Gln Met Leu Asn Val Leu Gly Asp His Gln Gly Ala
        50 55 60
Leu Gln Ile Val Lys Glu Ile Ile Asn Glu Glu Ala Ala Gln Trp Asp
65 70 75 80
Val Thr His Pro Leu Pro Ala Gly Pro Leu Pro Ala Gly Gln Leu Arg
                                85 90 95
Asp Pro Arg Gly Ser Asp Ile Ala Gly Thr Thr Ser Ser Val Gln Glu
                        100 105 110
Gln Leu Glu Trp Ile Tyr Thr Ala Asn Pro Arg Val Asp Val Gly Ala
                115 120 125
Ile Tyr Arg Arg Trp Ile Ile Leu Gly Leu Gln Lys Cys Val Lys Met
        130 135 140
Tyr Asn Pro Val Ser Val Leu Asp Ile Arg Gln Gly Pro Lys Glu Pro
145 150 155 160
Phe Lys Asp Tyr Val Asp Arg Phe Tyr Lys Ala Ile Arg Ala Glu Gln
                                165 170 175
Ala Ser Gly Glu Val Lys Gln Trp Met Thr Glu Ser Leu Leu Ile Gln
                        180 185 190
Asn Ala Asn Pro Asp Cys Lys Val Ile Leu Lys Gly Leu Gly Met His
                195 200 205
Pro Thr Leu Glu Glu Met Leu Thr Ala Cys Gln Gly Val Gly Gly Pro
        210 215 220
Ser Tyr Lys Ala Lys Val Met
225 230
SEQ ID NO: 24 p8 protein sequence
Val Gln Gln Gly Gly Pro Lys Arg Gln Arg Pro Pro Leu Arg Cys Tyr
1 5 10 15
Asn Cys Gly Lys Phe Gly His Met Gln Arg Gln Cys Pro Glu Pro Arg
                        20 25 30
Lys Thr Lys Cys Leu Lys Cys Gly Lys Leu Gly His Leu Ala Lys Asp
                35 40 45
Cys Arg Gly Gln Val Asn
        50
SEQ ID NO: 25 Protease sequence
Phe Glu Leu Pro Leu Trp Arg Arg Pro Ile Lys Thr Val Tyr Ile Glu
1 5 10 15
Gly Val Pro Ile Lys Ala Leu Leu Asp Thr Gly Ala Asp Asp Thr Ile
                        20 25 30
Ile Lys Glu Asn Asp Leu Gln Leu Ser Gly Pro Trp Arg Pro Lys Ile
                35 40 45
Ile Gly Gly Ile Gly Gly Gly Leu Asn Val Lys Glu Tyr Asn Asp Arg
        50 55 60
Glu Val Lys Ile Glu Asp Lys Ile Leu Arg Gly Thr Ile Leu Leu Gly
65 70 75 80
Ala Thr Pro Ile Asn Ile Ile Gly Arg Asn Leu Leu Ala Pro Ala Gly
                                85 90 95
Ala Arg Leu Val Met
                        100
SEQ ID NO: 26 p51 protein sequence
Gly Gln Leu Ser Glu Lys Ile Pro Val Thr Pro Val Lys Leu Lys Glu
1 5 10 15
Gly Ala Arg Gly Pro Cys Val Arg Gln Trp Pro Leu Ser Lys Glu Lys
                        20 25 30
Ile Glu Ala Leu Gln Glu Ile Cys Ser Gln Leu Glu Gln Glu Gly Lys
                35 40 45
Ile Ser Arg Val Gly Gly Glu Asn Ala Tyr Asn Thr Pro Ile Phe Cys
        50 55 60
Ile Lys Lys Lys Asp Lys Ser Gln Trp Arg Met Leu Val Asp Phe Arg
65 70 75 80
Glu Leu Asn Lys Ala Thr Gln Asp Phe Phe Glu Val Gln Leu Gly Ile
                                85 90 95
Pro His Pro Ala Gly Leu Arg Lys Met Arg Gln Ile Thr Val Leu Asp
                        100 105 110
Val Gly Asp Ala Tyr Tyr Ser Ile Pro Leu Asp Pro Asn Phe Arg Lys
                115 120 125
Tyr Thr Ala Phe Thr Ile Pro Thr Val Asn Asn Gln Gly Pro Gly Ile
        130 135 140
Arg Tyr Gln Phe Asn Cys Leu Pro Gln Gly Trp Lys Gly Ser Pro Thr
145 150 155 160
Ile Phe Gln Asn Thr Ala Ala Ser Ile Leu Glu Glu Ile Lys Arg Asn
                                165 170 175
Leu Pro Ala Leu Thr Ile Val Gln Tyr Met Asp Asp Leu Trp Val Gly
                        180 185 190
Ser Gln Glu Asn Glu His Thr His Asp Lys Leu Val Glu Gln Leu Arg
                195 200 205
Thr Lys Leu Gln Ala Trp Gly Leu Glu Thr Pro Glu Lys Lys Val Gln
        210 215 220
Lys Glu Pro Pro Tyr Glu Trp Met Gly Tyr Lys Leu Trp Pro His Lys
225 230 235 240
Trp Glu Leu Ser Arg Ile Gln Leu Glu Glu Lys Asp Glu Trp Thr Val
                                245 250 255
Asn Asp Ile Gln Lys Leu Val Gly Lys Leu Asn Trp Ala Ala Gln Leu
                        260 265 270
Tyr Pro Gly Leu Arg Thr Lys Asn Ile Cys Lys Leu Ile Arg Gly Lys
                275 280 285
Lys Asn Leu Leu Glu Leu Val Thr Trp Thr Pro Glu Ala Glu Ala Glu
        290 295 300
Tyr Ala Glu Asn Ala Glu Ile Leu Lys Thr Glu Gln Glu Gly Thr Tyr
305 310 315 320
Tyr Lys Pro Gly Ile Pro Ile Arg Ala Ala Val Gln Lys Leu Glu Gly
                                325 330 335
Gly Gln Trp Ser Tyr Gln Phe Lys Gln Glu Gly Gln Val Leu Lys Val
                        340 345 350
Gly Lys Tyr Thr Lys Gln Lys Asn Thr His Thr Asn Glu Leu Arg Thr
                355 360 365
Leu Ala Gly Leu Val Gln Lys Ile Cys Lys Glu Ala Leu Val Ile Trp
        370 375 380
Gly Ile Leu Pro Val Leu Glu Leu Pro Ile Glu Arg Glu Val Trp Glu
385 390 395 400
Gln Trp Trp Ala Asp Tyr Trp Gln Val Ser Trp Ile Pro Glu Trp Asp
                                405 410 415
Phe Val Ser Thr Pro Pro Leu Leu Lys Leu Trp Tyr Thr Leu Thr Lys
                        420 425 430
Glu Pro Ile Pro Lys Glu Asp Val Tyr
                435 440
SEQ ID NO: 27 p15 protein sequence
Tyr Val Asp Gly Ala Cys Asn Arg Asn Ser Lys Glu Gly Lys Ala Gly
1 5 10 15
Tyr Ile Ser Gln Tyr Gly Lys Gln Arg Val Glu Thr Leu Glu Asn Thr
                        20 25 30
Thr Asn Gln Gln Ala Glu Leu Thr Ala Ile Lys Met Ala Leu Glu Asp
                35 40 45
Ser Gly Pro Asn Val Asn Ile Val Thr Asp Ser Gln Tyr Ala Met Gly
        50 55 60
Ile Leu Thr Ala Gln Pro Thr Gln Ser Asp Ser Pro Leu Val Glu Gln
65 70 75 80
Ile Ile Ala Leu Met Ile Gln Lys Gln Gln Ile Tyr Leu Gln Trp Val
                                85 90 95
Pro Ala His Lys Gly Ile Gly Gly Asn Glu Glu Ile Asp Lys Leu Val
                        100 105 110
Ser Lys Gly Ile Arg Arg Val Leu
                115 120
SEQ ID NO: 28 p31 protein sequence
Phe Leu Glu Lys Ile Glu Glu Ala Gln Glu Glu His Glu Arg Tyr His
1 5 10 15
Asn Asn Trp Lys Asn Leu Ala Asp Thr Tyr Gly Leu Pro Gln Ile Val
                        20 25 30
Ala Lys Glu Ile Val Ala Met Cys Pro Lys Cys Gln Ile Lys Gly Glu
                35 40 45
Pro Val His Gly Gln Val Asp Ala Ser Pro Gly Thr Trp Gln Met Asp
        50 55 60
Cys Thr His Leu Glu Gly Lys Val Val Ile Val Ala Val His Val Ala
65 70 75 80
Ser Gly Phe Ile Glu Ala Glu Val Ile Pro Arg Glu Thr Gly Lys Glu
                                85 90 95
Thr Ala Lys Phe Leu Leu Lys Ile Leu Ser Arg Trp Pro Ile Thr Gln
                        100 105 110
Leu His Thr Asp Asn Gly Pro Asn Phe Thr Ser Gln Glu Val Ala Ala
                115 120 125
Ile Cys Trp Trp Gly Lys Ile Glu His Thr Thr Gly Ile Pro Tyr Asn
        130 135 140
Pro Gln Ser Gln Gly Ser Ile Glu Ser Met Asn Lys Gln Leu Lys Glu
145 150 155 160
Ile Ile Gly Lys Ile Arg Asp Asp Cys Gln Tyr Thr Glu Thr Ala Val
                                165 170 175
Leu Met Ala Cys His Ile His Asn Phe Lys Arg Lys Gly Gly Ile Gly
                        180 185 190
Gly Gln Thr Ser Ala Glu Arg Leu Ile Asn Ile Ile Thr Thr Gln Leu
                195 200 205
Glu Ile Gln His Leu Gln Thr Lys Ile Gln Lys Ile Leu Asn Phe Arg
        210 215 220
Val Tyr Tyr Arg Glu Gly Arg Asp Pro Val Trp Lys Gly Pro Ala Gln
225 230 235 240
Leu Ile Trp Lys Gly Glu Gly Ala Val Val Leu Lys Asp Gly Ser Asp
                                245 250 255
Leu Lys Val Val Pro Arg Arg Lys Ala Lys Ile Ile Lys Asp Tyr Glu
                        260 265 270
Pro Lys Gln Arg Val Gly Asn Glu Gly Asp Val Glu Gly Thr Arg Gly
                275 280 285
Ser Asp Asn
        290
SEQ ID NO: 29 Gag protein
Met Gly Ala Ala Thr Ser Ala Leu Asn Arg Arg Gln Leu Asp Gln Phe
1 5 10 15
Glu Lys Ile Arg Leu Arg Pro Asn Gly Lys Lys Lys Tyr Gln Ile Lys
                        20 25 30
His Leu Ile Trp Ala Gly Lys Glu Met Glu Arg Phe Gly Leu His Glu
                35 40 45
Arg Leu Leu Glu Thr Glu Glu Gly Cys Lys Arg Ile Ile Glu Val Leu
        50 55 60
Tyr Pro Leu Glu Pro Thr Gly Ser Glu Gly Leu Lys Ser Leu Phe Asn
65 70 75 80
Leu Val Cys Val Leu Tyr Cys Leu His Lys Glu Gln Lys Val Lys Asp
                                85 90 95
Thr Glu Glu Ala Val Ala Thr Val Arg Gln His Cys His Leu Val Glu
                        100 105 110
Lys Glu Lys Ser Ala Thr Glu Thr Ser Ser Gly Gln Lys Lys Asn Asp
                115 120 125
Lys Gly Ile Ala Ala Pro Pro Gly Gly Ser Gln Asn Phe Pro Ala Gln
        130 135 140
Gln Gln Gly Asn Ala Trp Val His Val Pro Leu Ser Pro Arg Thr Leu
145 150 155 160
Asn Ala Trp Val Lys Ala Val Glu Glu Lys Lys Phe Gly Ala Glu Ile
                                165 170 175
Val Pro Met Phe Gln Ala Leu Ser Glu Gly Cys Thr Pro Tyr Asp Ile
                        180 185 190
Asn Gln Met Leu Asn Val Leu Gly Asp His Gln Gly Ala Leu Gln Ile
                195 200 205
Val Lys Glu Ile Ile Asn Glu Glu Ala Ala Gln Trp Asp Val Thr His
        210 215 220
Pro Leu Pro Ala Gly Pro Leu Pro Ala Gly Gln Leu Arg Asp Pro Arg
225 230 235 240
Gly Ser Asp Ile Ala Gly Thr Thr Ser Ser Val Gln Glu Gln Leu Glu
                                245 250 255
Trp Ile Tyr Thr Ala Asn Pro Arg Val Asp Val Gly Ala Ile Tyr Arg
                        260 265 270
Arg Trp Ile Ile Leu Gly Leu Gln Lys Cys Val Lys Met tyr Asn Pro
                275 280 285
Val Ser Val Leu Asp Ile Arg Gln Gly Pro Lys Glu Pro Phe Lys Asp
        290 295 300
Tyr Val Asp Arg Phe Tyr Lys Ala Ile Arg Ala Glu Gln Ala Ser Gly
305 310 315 320
Glu Val Lys Gln Trp Met Thr Glu Ser Leu Leu Ile Gln Asn Ala Asn
                                325 330 335
Pro Asp Cys Lys Val Ile Leu Lys Gly Leu Gly Met His Pro Thr Leu
                        340 345 350
Glu Glu Met Leu Thr Ala Cys Gln Gly Val Gly Gly Pro Ser Tyr Lys
                355 360 365
Ala Lys Val Met Ala Glu Met Met Gln Thr Met Gln Asn Gln Asn Met
        370 375 380
Val Gln Gln Gly Gly Pro Lys Arg Gln Arg Pro Pro Leu Arg Cys Tyr
385 390 395 400
Asn Cys Gly Lys Phe Gly His Met Gln Arg Gln Cys Pro Glu Pro Arg
                                405 410 415
Lys Thr Lys Cys Leu Lys Cys Gly Lys Leu Gly His Leu Ala Lys Asp
                        420 425 430
Cys Arg Gly Gln Val Asn Phe Leu Gly Tyr Gly Arg Trp Met Gly Ala
                435 440 445
Lys Pro Arg Asn Phe Pro Ala Ala Thr Leu Gly Ala Glu Pro Ser Ala
        450 455 460
Pro Pro Pro Pro Ser Gly Thr Thr Pro Tyr Asp Pro Ala Lys Lys Leu
465 470 475 480
Leu Gln Gln Tyr Ala Glu Lys Gly Lys Gln Leu Arg Glu Gln Lys Arg
                                485 490 495
Asn Pro Pro Ala Met Asn Pro Asp Trp Thr Glu Gly Tyr Ser Leu Asn
                        500 505 510
Ser Leu Phe Gly Glu Asp Gln
                515
SEQ ID NO: 30 Pol protein
Met Ser Lys Val Trp Lys Ile Gly Thr pro Ser Lys Arg Leu gln Gly
1 5 10 15
Thr Gly Glu Phe Phe Arg Val Trp Thr Val Asp Gly gly Lys Thr Glu
                        20 25 30
Lys Phe Ser Arg Arg Tyr Ser Trp Ser Gly Thr Glu Cys Ala Ser Ser
                35 40 45
Thr Glu Arg His His Pro Ile Arg Pro Ser Lys Glu Ala Pro Ala Ala
        50 55 60
Ile Cys Arg Glu Arg Glu Thr Thr Glu Gly Ala Lys Glu Glu Ser Thr
65 70 75 80
Gly Asn Glu Ser Gly Leu Asp Arg Gly Ile Phe Phe Glu Leu Pro Leu
                                85 90 95
Trp Arg Arg Pro Ile Lys Thr Val Tyr Ile Glu Gly Val Pro Ile Lys
                        100 105 110
Ala Leu Leu Asp Thr Gly Ala Asp Asp Thr Ile Ile Lys Glu Asn Asp
                115 120 125
Leu Gln Leu Ser Gly Pro Trp Arg Pro Lys Ile Ile Gly Gly Ile Gly
        130 135 140
Gly Gly Leu Asn Val Lys Glu Tyr Asn Asp Arg Glu Val Lys Ile Glu
145 150 155 160
Asp Lys Ile Leu Arg Gly Thr Ile Leu Leu Gly Ala Thr Pro Ile Asn
                                165 170 175
Ile Ile Gly Arg Asn Leu Leu Ala Pro Ala Gly Ala Arg Leu Val Met
                        180 185 190
Gly Gln Leu Ser Glu Lys Ile Pro Val Thr Pro Val Lys Leu Lys Glu
                195 200 205
Gly Ala Arg Gly Pro Cys Val Arg Gln Trp Pro Leu Ser Lys Glu Lys
        210 215 220
Ile Glu Ala Leu Gln Glu Ile Cys Ser Gln Leu Glu Gln Glu Gly Lys
225 230 235 240
Ile Ser Arg Val Gly Gly Glu Asn Ala Tyr Asn Thr Pro Ile Phe Cys
                                245 250 255
Ile Lys Lys Lys Asp Lys Ser Gln Trp Arg Met Leu Val Asp Phe Arg
                        260 265 270
Glu Leu Asn Lys Ala Thr Gln Asp Phe Phe Glu Val Gln Leu Gly Ile
                275 280 285
Pro His Pro Ala Gly Leu Arg Lys Met Arg Gln Ile Thr Val Leu Asp
        290 295 300
Val Gly Asp Ala Tyr Tyr Ser Ile Pro Leu Asp Pro Asn Phe Arg Lys
305 310 315 320
Tyr Thr Ala Phe Thr Ile Pro Thr Val Asn Asn Gln Gly Pro Gly Ile
                                325 330 335
Arg Tyr Gln Phe Asn Cys Leu Pro Gln Gly Trp Lys Gly Ser Pro Thr
                        340 345 350
Ile Phe Gln Asn Thr Ala Ala Ser Ile Leu Glu Glu Ile Lys Arg Asn
                355 360 365
Leu Pro Ala Leu Thr Ile Val Gln Tyr Met Asp Asp Leu Trp Val Gly
        370 375 380
Ser Gln Glu Asn Glu His Thr His Asp Lys Leu Val Glu Gln Leu Arg
385 390 395 400
Thr Lys Leu Gln Ala Trp Gly Leu Glu thr Pro Glu Lys Lys Val Gln
                                405 410 415
Lys Glu Pro Pro Tyr Glu Trp Met Gly Tyr Lys Leu Trp Pro His Lys
                        420 425 430
Trp Glu Leu Ser Arg Ile Gln Leu Glu Glu Lys Asp Glu Trp Thr Val
                435 440 445
Asn Asp Ile Gln Lys Leu Val Gly Lys Leu Asn Trp Ala Ala Gln Leu
        450 455 460
Tyr Pro Gly Leu Arg Thr Lys Asn Ile Cys Lys Leu Ile Arg Gly Lys
465 470 475 480
Lys Asn Leu Leu Glu Leu Val Thr Trp Thr Pro Glu Ala Glu Ala Glu
                                485 490 495
Tyr Ala Glu Asn Ala Glu Ile Leu Lys Thr Glu Gln Glu Gly Thr Tyr
                        500 505 510
Tyr Lys Pro Gly Ile Pro Ile Arg Ala Ala Val Gln Lys Leu Glu Gly
                515 520 525
Gly Gln Trp Ser Tyr Gln Phe Lys Gln Glu Gly Gln Val Leu Lys Val
        530 535 540
Gly Lys Tyr Thr Lys Gln Lys Asn Thr His Thr Asn Glu Leu Arg Thr
545 550 555 560
Leu Ala Gly Leu Val Gln Lys Ile Cys Lys Glu Ala Leu Val Ile Trp
                                565 570 575
Gly Ile Leu Pro Val Leu Glu Leu Pro Ile Glu Arg Glu Val Trp Glu
                        580 585 590
Gln Trp Trp Ala Asp Tyr Trp Gln Val Ser Trp Ile Pro Glu Trp Asp
                595 600 605
Phe Val Ser Thr Pro Pro Leu Leu Lys Leu Trp Tyr Thr Leu Thr Lys
        610 615 620
Glu Pro Ile Pro Lys Glu Asp Val Tyr Tyr Val Asp Gly Ala Cys Asn
625 630 635 640
Arg Asn Ser Lys Glu Gly Lys Ala Gly Tyr Ile Ser Gln Tyr Gly Lys
                                645 650 655
Gln Arg Val Glu Thr Leu Glu Asn Thr Thr Asn Gln Gln Ala Glu Leu
                        660 665 670
Thr Ala Ile Lys Met Ala Leu Glu Asp Ser Gly Pro Asn Val Asn Ile
                675 680 685
Val Thr Asp Ser Gln Tyr Ala Met Gly Ile Leu Thr Ala Gln Pro Thr
        690 695 700
Gln Ser Asp Ser Pro Leu Val Glu Gln Ile Ile Ala Leu Met Ile Gln
705 710 715 720
Lys Gln Gln Ile Tyr Leu Gln Trp Val Pro Ala His Lys Gly Ile Gly
                                725 730 735
Gly Asn Glu Glu Ile Asp Lys Leu Val Ser Lys Gly Ile Arg Arg Val
                        740 745 750
Leu Phe Leu Glu Lys Ile Glu Glu Ala gln Glu Glu His Glu Arg Tyr
                755 760 765
His Asn Asn Trp Lys Asn Leu Ala Asp Thr Tyr Gly Leu Pro Gln Ile
        770 775 780
Val Ala Lys Glu Ile Val Ala Met Cys Pro Lys Cys Gln Ile Lys Gly
785 790 795 800
Glu Pro Val His Gly Gln Val Asp Ala Ser Pro Gly Thr Trp Gln Met
                                805 810 815
Asp Cys Thr His Leu Glu Gly Lys Val Val Ile Val Ala Val His Val
                        820 825 830
Ala Ser Gly Phe Ile Glu Ala Glu Val Ile Pro Arg Glu Thr Gly Lys
                835 840 845
Glu Thr Ala Lys Phe Leu Leu Lys Ile Leu Ser Arg Trp pro Ile Thr
        850 855 860
Gln Leu His Thr Asp Asn Gly Pro Asn Phe Thr Ser Gln Glu Val Ala
865 870 875 880
Ala Ile Cys Trp Trp Gly Lys Ile Glu His Thr Thr Gly Ile Pro Tyr
                                885 890 895
Asn Pro Gln Ser Gln Gly Ser Ile Glu Ser Met Asn Lys Gln Leu Lys
                        900 905 910
Glu Ile Ile Gly Lys Ile Arg Asp Asp Cys Gln Tyr Thr Glu Thr Ala
                915 920 925
Val Leu Met Ala Cys His Ile His Asn Phe Lys Arg Lys Gly Gly Ile
        930 935 940
Gly Gly Gln Thr Ser Ala Glu Arg Leu Ile Asn Ile Ile Thr Thr Gln
945 950 955 960
Leu Glu Ile Gln His Leu Gln Thr Lys Ile Gln LYs Ile Leu Asn Phe
                                965 970 975
Arg Val Tyr Tyr Arg Glu Gly Arg Asp Pro Val Trp Lys Glu Pro Ala
                        980 985 990
Gln Leu Ile Trp Lys Gly Glu Gly Ala Val Val Leu Lys Asp Gly Ser
                995 1000 1005
Asp Leu Lys Val Val Pro Arg Arg Lys Ala Lys Ile Ile Lys Asp
        1010 1015 1020
Tyr Glu Pro Lys Gln Arg Val Gly Asn Glu Gly Asp Val Glu Gly
        1025 1030 1035
Thr Arg Gly Ser Asp Asn
        1040
```

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Transduction Efficiency in Human Bronchial Epithelial Cells (HBEC, F508del/F508del, Class II) Grown at an Air Liquid Interface (ALI) Culture

To analyse the transduction efficiency of HBECs (basal cells) subsequently grown at an ALI culture, cells were transduced in submerged culture with rSIV.F/HN, expressing GFP (vFM107, rSIV.F/HN-GFP) at different multiplicities of infection (MOI) of 3, 10, 30 and 90, followed by airlift at 2 days post transduction (FIG. 2A). 3-4 weeks after airlift, cells were analysed in their fully differentiated state. At day 21 after airlift the percentage of GFP positive cells was measured by flow cytometry. Cells transduced at MOIs 3, 10, 30 and 90 resulted in significant (p<0.001, p<0.0001) dose-dependent increase in transduction efficiency: 7.8±1.1%, 17.6±1.0%, 25.8±1.8 and 28.3±1.8% of GFP positive cells respectively (FIG. 2B).

Next, the cellular profile of the ALIs derived from the transduced basal cells was examined by applying immunofluorescence staining for different epithelial cell markers at day 28 post transduction. Co-localisation of GFP with ACTUB (ciliated cells), KRT5 (basal cells), SCGB1A1 (club cells) and MUC5AC (goblet cells) was detectable, confirming that rSIV.F/HN produced expression in multiple cell types subsequent to basal cell transduction (FIG. 2C). These findings were further confirmed by flow cytometry measurement of GFP positive cell percentages of KRT5⁺, ACTU13⁺ and SCGB1A1⁺ cells. At MOI 10 around 20% of cells of each cell population were transduced with the virus, whilst at MOI 30 this increased to 30-40% (FIG. 2D).

Additionally, average integrations in genome (vector copy numbers (VCN)) were analysed in DNA samples from ALI cultures, which were independently transduced with either GFP- (vGM107) or CFTR- (vGM058) expressing rSIV.F/HN. Bulk DNA analysis showed a dose related increase in VCN for both GFP- and CFTR-expressing rSIV.F/HN (30.0±5.2/35.8±4.0; 60.3±11.9/58.6±6.3; 124.2±25.2/87.2±10.5 copies/ng DNA in cells transduced at MOI 3, 10, 90 with GFP/CFTR-expressing rSIV.F/HN). No difference in VCN was observed between GFP- and CFTR-expressing rSIV.F/HN at any of the MOIs analysed (FIG. 2E).

Vector-derived Woodchuck hepatitis post-transcriptional regulatory element (WPRE) mRNA expression was also analyzed on sorted single cells of ALIs transduced at MOI 10 and again showed no difference in WPRE expression between cells transduced with GFP- (vGM107) or CFTR-expressing (vGM058) rSIV.F/HN (FIG. 2F). High variability in number of WPRE copies per cell has been observed suggesting that some cells could be identified as high expressors and other cells as medium/low expressors. Both analyses (FIG. 2E, F) suggest that the transduction efficiencies of GFP-expressing rSIV.F/HN and CFTR-expressing rSIV.F/HN are comparable.

Based on these data, transduction rates of GFP-expressing rSIV.F/HN were used as a surrogate readout to estimate transduction levels of CFTR-expressing rSIV.F/HN, thereby enabling correlative analyses between transduction levels and the degree of functional CFTR restoration.

### Example 2—Transduction of CF ALI Cultures (F508deI/F508deI, Class II) with rSIV.F/HN-CFTR (vGM058) Results in Transgenic CFTR Expression, Restoration of CFTR Chloride Current and Increased Ciliary Beat Frequency

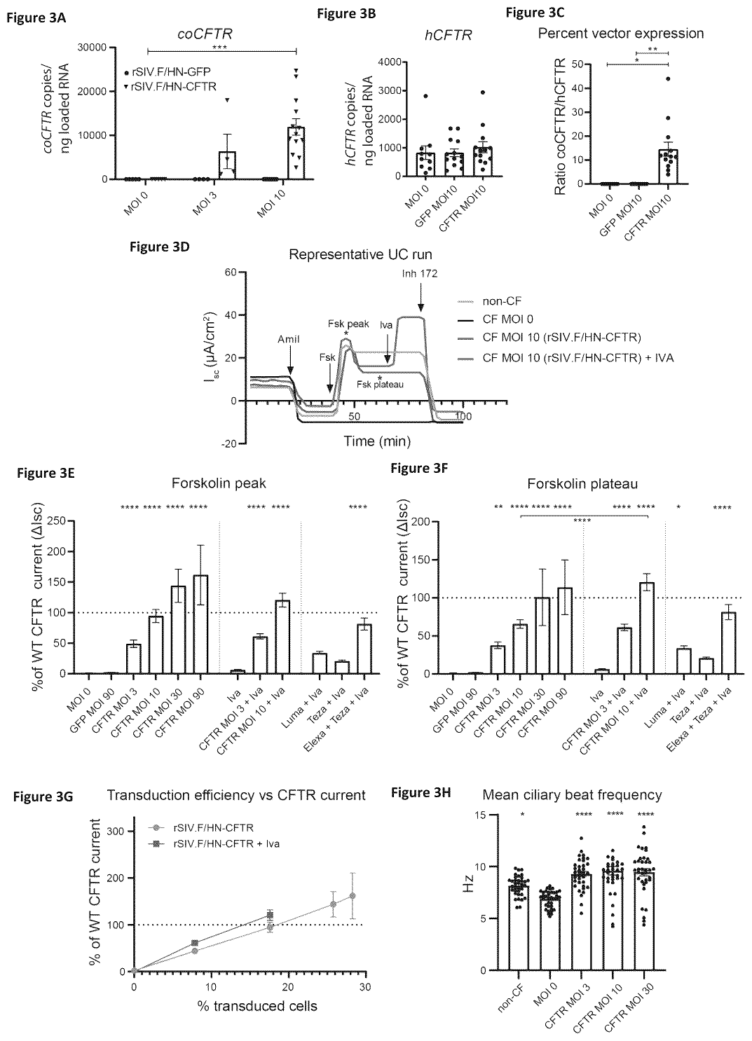

To determine whether rSIV.F/HN-CFTR transduced CF ALIs can efficiently produce vector-derived codon optimised CFTR mRNA (coCFTR), quantitative ddPCR analysis was performed. Dose-dependent coCFTR expression was observed in cells transduced with rSIV.F/HN-CFTR (vGM058) at MOI 3 and 10, while no expression was observed in cells transduced with rSIV.F/HN-GFP (vGM107) (FIG. 3A). Expression of endogenous CFTR was also analysed and no differences between non-transduced samples, samples transduced with rSIV.F/HN-GFP and samples transduced with rSIV.F/HN-CFTR at MOI 10 were observed (FIG. 3B). mRNA expression of coCFTR was 10 times higher than expression of endogenous CFTR (FIG. 3C) suggesting that a low number of transduced CFTR high-expressing cells (17% for MOI 10) are enough to restore CFTR expression higher than endogenous levels.

To analyse the functionality of rSIV.F/HN-CFTR-expressed channels, CFTR-mediated chloride current was measured in an Ussing chamber using rSIV.F/HN-CFTR (vGM058) transduced ALI cultures. First, sodium channels (ENaC) were blocked using amiloride, followed by stimulation of CFTR using forskolin and the change in short circuit current (Δlsc) was calculated (both peak and plateau values). In some experiments, ivacaftor, a small molecule CFTR potentiator which increases channel open probability, was added for additional stimulation of CFTR current. Finally, the chloride current was blocked with CFTR-Inhibitor 172 (FIG. 3D).

As expected, non-transduced cells (MOI 0), or cells transduced with rSIV.F/HN-GFP did not respond to forskolin or the CFTR-inhibitor confirming the absence of functional CFTR channels. In contrast, a dose-related increase in CFTR chloride current was observed in ALIs transduced with rSIV.F/HN-CFTR. At an MOI of 3, there was restoration of 49±6% (peak, p<0.0001) and 38±4% (plateau, p<0.01) of the non-CF chloride current. When the rSIV.F/HN-CFTR (MOI 3) was combined with the potentiator ivacaftor, there was a significant increase in stimulation of chloride current (61±4% for both peak and plateau). With an increased MOI of 10, we observed significantly higher restoration (94±11% peak, 66±6% plateau, p<0.0001) of the non-CF chloride current; a combination of MOI 10 and ivacaftor led to further increase in restoration (121±11% for both peak and plateau). Higher MOIs (30 and 90) resulted in further significant increase: 144±27 and 162±49% of restoration (peak, p<0.0001) and 101±37 and 114±36% (plateau, p<0.0001) respectively.

Thus, rSIV.F/HN-CFTR is able to completely restore the CFTR-related chloride current to non-CF values and a combination of rSIV.F/HN-CFTR with ivacaftor amplifies this effect of gene therapy by 1.3-1.8-fold. These data compare favourably with current approved modulator therapies which were also assessed. Thus, treatment with lumacaftor+ivacaftor and tezacaftor+ivacaftor produced 34% and 21% of CFTR current restoration respectively, whilst treatment with elexacaftor+tezacaftor+ivacaftor resulted in 81% of restoration (FIG. 3E, F; Table 1).

TABLE 1
Ussing chamber results for Class II F508del/F508del CF ALI cultures transduced with rSIV.F/HN (vGM058)

| | | Dose | | | ΔCFTR- | ΔCFTR- | % of | % of |
|---|---|---|---|---|---|---|---|---|
| | Treatment | of LV | ΔFskₚₑₐₖ | ΔFskₚₗₐₜₑₐᵤ | inh172ₚₑₐₖ | inh172ₚₗₐₜₑₐᵤ | WT₍ₚₑₐₖ₎ | WT₍ₚₗₐₜₑₐᵤ₎ |
| Non-CF | — | — | 32.3 ± 3.5 | 31.8 ± 3.2 | −34.3 ± 3.4 | −33.6 ± 3.4 | 100 | 100 |
| (WT) | | | | | | | | |
| CF | — | — | −0.003 ± 0.1 | −0.003 ± 0.1 | −0.4 ± 0.1 | −0.4 ± 0.1 | — | — |
| | rSIV.F/HN-GFP | MOI 90 | −0.6 ± 0.07 | −0.6 ± 0.07 | −0.7 ± 0.1 | −0.7 ± 0.1 | — | — |
| | rSIV.F/HN-CFTR | MOI 3 | 14.1 ± 1.9 | 9.7 ± 1.2 | −16.8 ± 2.1 | −12.9 ± 1.5 | 49 | 38 |
| | | MOI 10 | 30.1 ± 3.6 | 18.1 ± 1.6 | −32.4 ± 3.6 | −22.5 ± 1.9 | 94 | 67 |
| | | MOI 30 | 38.2 ± 6.1 | 23.3 ± 6.7 | −49.4 ± 9.3 | −34.6 ± 12.8 | 144 | 103 |
| | | MOI 90 | 47.3 ± 14.6 | 33.9 ± 11.4 | −55.5 ± 16.8 | −39.1 ± 12.4 | 162 | 116 |
| | Iva | — | −0.1 ± 0.3 | −0.1 ± 0.3 | −2.1 ± 0.3 | −2.1 ± 0.3 | — | — |
| | rSIV.F/HN- | MOI 3 | 20.4 ± 1.6 | 20.4 ± 1.6 | −21.0 ± 1.5 | −21.0 ± 1.5 | 61 | 62 |
| | CFTR + Iva | MOI 10 | 35.3 ± 3.3 | 35.3 ± 3.3 | −41.4 ± 3.8 | −41.4 ± 3.8 | 121 | 123 |
| | Luma + Iva | — | 12.1 ± 1.1 | 12.1 ± 1.1 | −11.6 ± 1.1 | −11.6 ± 1.1 | 34 | 34 |
| | Teza + Iva | — | 7.4 ± 0.5 | 7.4 ± 0.5 | −7.1 ± 0.6 | −7.1 ± 0.6 | 21 | 21 |
| | Elexa + | — | 33.5 ± 0.5 | 33.5 ± 0.5 | −27.9 ± 3.4 | −27.9 ± 3.4 | 81 | 83 |
| | Teza + Iva | | | | | | | |

LV—lentivirus,
Fsk—forskolin,
CFTR-inh172—CFTR inhibitor 172,
Iva—ivacaftor,
Luma—lumacaftor,
Teza—tezacaftor,
Elexa—elexacaftor

Since the transduction efficiency of rSIV.F/HN-GFP and rSIV.F/HN-CFTR was shown to be similar (FIG. 2E, F), a correlative analysis between GFP-based transduction levels and the degree of CFTR chloride current restoration was assessed (FIG. 3G). Approximately 17% of transduced cells was sufficient to restore the CFTR chloride current to physiological levels. When rSIV.F/HN-CFTR was

used in combination with ivacaftor full restoration was achieved with approximately 14% transduced cells.

To investigate the downstream functional consequences of coCFTR expression, ciliary beat frequency (CBF) was measured as a surrogate readout of mucociliary clearance. A significant reduction in CBF in the CF ALIs (6.9±0.8 Hz in comparison to 8.2±1 Hz in non-CF ALIs, p<0.05) was demonstrated. Transduction with rSIV.F/HN-CFTR was able to restore CBF to non-CF values (9.3±1.5, 9.7±2.6 and 9.5±2.2 Hz for MOI 3, 10 and 30 respectively, all p>0.0001) (FIG. 3H).

### Example 3—Generation of CFTR Knockout hSABCi Cell Line as a CF Translational Class I Mutation Model and Transduction of CFTR Knockout Cells with rSIV.F/HN (vGM107, vGM058)

Class I CFTR null mutations result in complete absence of full length CFTR protein and are thus not amenable to functional correction by current CFTR modulators. Gene therapy provides an opportunity to establish a disease-modifying treatment for patients with all mutation types, including those homozygous for these null mutations. Primary airway epithelial cells derived from the small number of patients with Class I mutations are difficult to obtain. Thus, in order to enable functional characterization of rSIV.F/HN-CFTR in a Class I mutation background, a mutation model was generated via a CRISPR/Cas9 mediated bi-allelic CFTR knockout (KO) in exon 4 of the CFTR gene using the previously described immortalized human small airway basal cell line hSABCi-NS1.1 (hSABCi). This cell line maintains basal cells features (TP63+, KRT5+) after more than 200 cell division cycles and 70 passages.

When cultured in air liquid interface conditions, hSABCi cells consistently form tight junctions and differentiate into ciliated (ARL13B+), club (SCGB1A1+), goblet (MUC5AC+, MUC5B+), neuroendocrine (CHGA+), ionocyte (FOXI1+) and surfactant protein positive cells (SFTPA+, SFTPB+, SFTPD+). Additionally, this cell line has been validated for the presence of ENaC and CFTR channel activity. hSABCi cells were transfected with a complex of synthetic tracr:cr RNA and recombinant Cas9. To analyse editing efficiency, the edited locus was amplified by PCR and then subjected to Sanger sequencing. Editing efficiency was determined for selected single clones using an established bioinformatic procedure to generate inference of CRISPR editing (ICE) metrics (data not shown). The clone (clone 5) with the highest out-of-frame editing/knock out efficiency of 99.4% (data not shown) was chosen for further experiments.

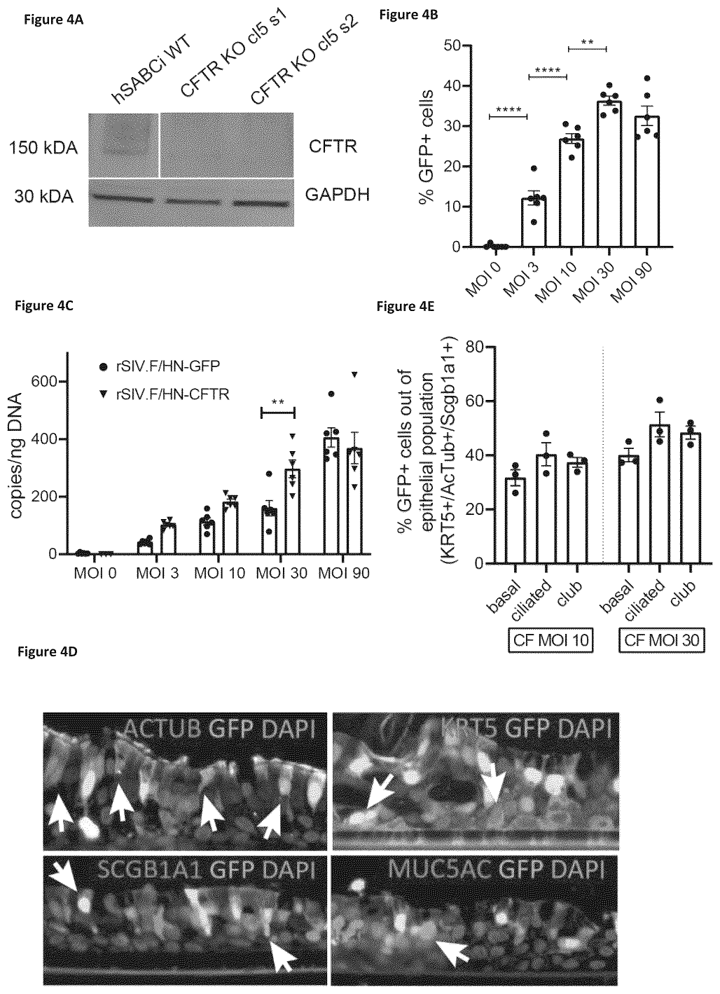

To characterize further the CFTR-KO phenotype, CFTR protein levels were analysed by Western blotting using monoclonal hCFTR antibody (R&D systems, Minneapolis, MN, US). A weak signal for the mature CFTR in the non-edited hSABCi cell line was observed, whereas no signal for the mature CFTR could be detected in CFTR KO cells (FIG. 4A), thereby confirming CFTR deficiency.

Next, the efficiency of lentiviral transduction in CFTR KO cells grown in ALI cultures was analysed. CFTR KO cells were transduced and analysed as for primary F508del/F508del cells. Flow cytometry analysis revealed that transduction with rSIV.F/HN-GFP (vGM107) at MOIs 3, 10, 30 and 90 resulted in average mean of 12.2±1.7 (p<0.0001), 26.9±1.2 (p<0.0001), 36.3±1.1 (p<0.01) and 32.6±2.4% of GFP+ cells respectively (FIG. 4B). Additionally, DNA VCN analysis of CFTR KO ALI cultures independently transduced with either rSIV.F/HN-GFP (vGM107) or rSIV.F/HN-CFTR (vGM058) showed no difference in VCN between rSIV.F/HN-GFP- and rSIV.F/HN-CFTR transduced cells at all MOIs analysed, apart from MOI 30 at which GFP-transduced cells showed a slight increase in VCN in comparison to CFTR-transduced cells (p<0.01, FIG. 4C). These data suggest that transduction efficiencies of rSIV.F/HN-GFP and rSIV.F/HN-CFTR are similar. Finally, rSIV.F/HN-GFP-mediated transduction of CFTR KO cells was analysed at the cellular level by immunofluorescence staining for different epithelial cell markers at day 28 post transduction. Co-localisation of GFP with ACTUB, KRT5, SCGB1A1 and MUC5AC was observed suggesting that rSIV.F/HN-GFP was able to transduce ciliated, basal, club and goblet cells respectively (FIG. 2E). Additionally, flow cytometry measurement of GFP+ cell percentages in KRT5+, ACTUB+ and SCGB1A1+ cells confirmed previous findings in Class II cells (Example 2). At an MOI 10 a mean of 16.5±1.8, 58±2.1 and 26.3±0.8% of ciliated, club and basal cells were transduced with the virus (FIG. 4E).

### Example 4—Transduction of CFTR KO ALI Cultures (Class I) with rSIV.F/HN-CFTR (vGM058) Results in Expression of coCFTR mRNA and CFTR Current Restoration, while Ion Channel Modulators Fail to Restore CFTR Current

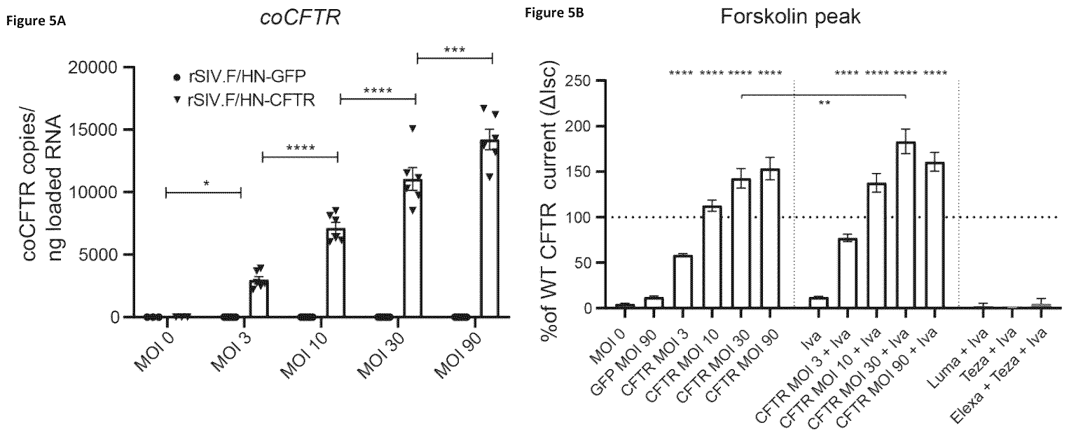

Codon optimised CFTR mRNA expression was analyzed in CFTR KO ALIs transduced with rSIV.F/HN-CFTR (vGM058). Dose-dependent increase in coCFTR expression was observed in cells transduced with rSIV.F/HN-CFTR in comparison to non-transduced cells, while no coCFTR expression was observed in cells transduced with rSIV.F/HN-GFP at all MOIs as expected (FIG. 5A).

Functional analysis via Ussing chamber measurements showed a dose-related increase in CFTR current in CFTR KO ALIs (class I) transduced with rSIV.F/HN-CFTR (vGM058) (FIGS. 5B, C). Untransduced cells did not show any activation of CFTR current, supporting functional success of the genome editing. Cells transduced with rSIV.F/HN-CFTR at an MOI 3 resulted in 58±2% of CFTR current restoration at forskolin peak and 54±2% of restoration at plateau (both p<0.0001). A combination of MOI 3 with the potentiator drug ivacaftor increased this effect to 77±4%. An MOI 10 resulted in 112±6% of CFTR current restoration at a peak (103±6% at plateau, both p<0.0001) and 138±10% in combination with ivacaftor (p<0.05 for plateau). MOIs of 30 and 90 resulted in 142±11% and 153±12% of restoration at a peak, and 135±9% and 149±11% at plateau respectively (all p<0.0001); combination with ivacaftor increased these effects to 183±13% (p<0.01 for peak and p<0.001 for plateau) and 161±10% respectively (FIG. 5C). Thus, combination of rSIV.F/HN-CFTR with ivacaftor increased the effect of gene therapy by 1.3-1.4-fold. In addition, clinically used CFTR modulators were analysed and as expected showed no evidence of CFTR restoration (FIGS. 5B, C; Table 2.

Since the transduction efficiencies of rSIV.F/HN-GFP and rSIV.F/HN-CFTR are similar (FIG. 4C), the correlation between percentage of transduced cells and degree of CFTR current restoration in CFTR KO cells was inferred (FIG. 5D). Around 23% of cells transduced with rSIV.F/HN-CFTR was sufficient to restore CFTR current to the levels of the parental hSABCi cell line. When rSIV.F/HN-CFTR was used in combination with ivacaftor full restoration was achieved with around 17% of transduced cells.

### Example 5—Comparison of Pre-Clinical Candidate vGM058 and Clinical Candidate vGM244 (BI 3720931

To compare pre-clinical candidate virus (vGM058) with the clinical candidate virus (vGM244) class II HBECs were transduced with rSIV.F/HN, expressing GFP (vGM107) and rSIV.F/HN, expressing CFTR (vGM058 and vGM244) with MOIs of 3 and 10. At day 21 after airlift cells were collected and average integrations in genome (vector copy numbers (VCN)) were analysed in DNA samples from ALI cultures transduced at MOI 10.

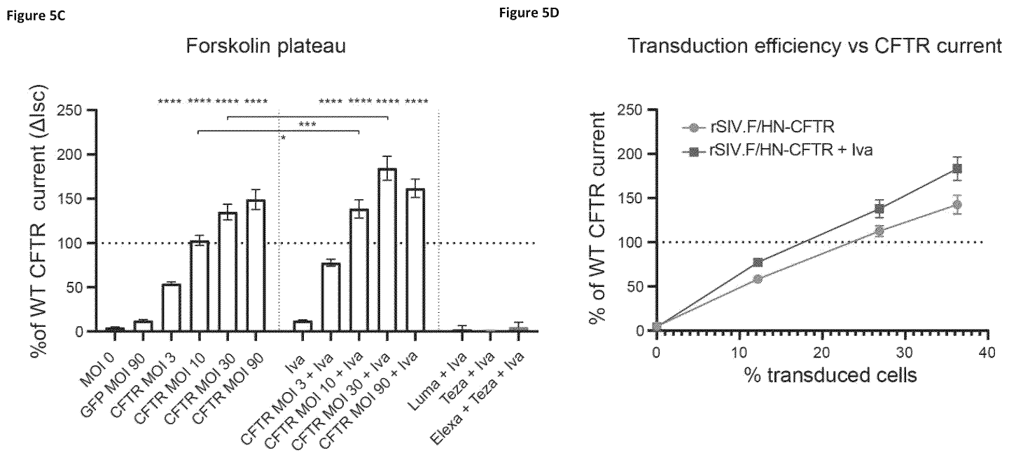

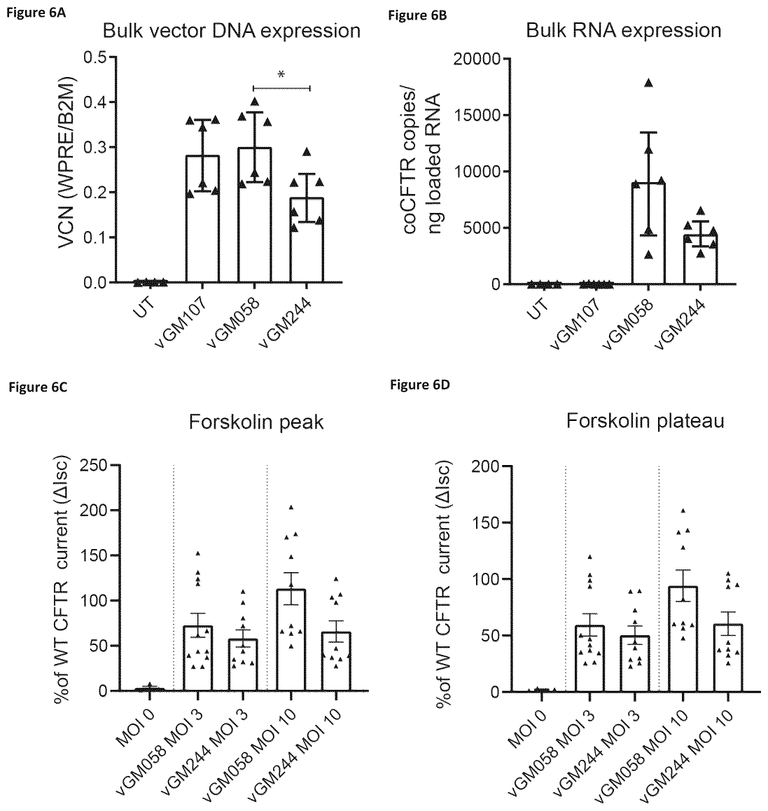

No difference in VCN levels was observed between vGM107 and vGM058. However, cells transduced with vGM244 had significantly (p<0.05) lower VCN levels in comparison to cells transduced with vGM058 (FIG. 6A). Codon optimized CFTR mRNA expression was also analysed. Cells transduced with vGM244 had a lower, non-significant (p=0.13) decrease in coCFTR expression in comparison to cells transduced with vGM058 (FIG. 6B).

To compare the functionality of vGM058 and vGM244, CFTR-mediated chloride current was measured in an Ussing chamber using class II ALI cultures transduced with both vGM058 and vGM244. For both MOI 3 and 10, levels of functional correction for the vGM244 virus were lower, however non-significant, than for the vGM058 virus (FIGS. 6C and D).

Any difference in VCN, coCFTR and Ussing chamber functional data between vGM058 and vGM244 may be attributed to titer variability of the virus due to different protocols of titer measurement between Oxford Biomedica (source of vGM244) and Oxford University (source of vGM058).

TABLE 2
Ussing chamber results for Class I CFTR KO ALI cultures transduced with rSIV.F/HN (vGM058)

| | | Dose | | | ΔCFTR- | ΔCFTR- | % of | % of |
|---|---|---|---|---|---|---|---|---|
| | Treatment | of LV | ΔFskₚₑₐₖ | ΔFsk ₚₗₐₜₑₐᵤ | inh172ₚₑₐₖ | inh172ₚₗₐₜₑₐᵤ | WT₍ₚₑₐₖ₎ | WT₍ₚₗₐₜₑₐᵤ₎ |
| Non-CF | — | — | 33.6 ± 0.7 | 33.6 ± 0.7 | −31.7 ± 1.1 | −31.7 ± 1.1 | 100 | 100 |
| (WT) | | | | | | | | |
| CF | — | — | −0.5 ± 0.1 | −0.5 ± 0.1 | −1.1 ± 0.2 | −1.1 ± 0.2 | — | — |
| | rSIV.F/HN-GFP | MOI 90 | −1 ± 0.7 | −1 ± 0.7 | −3.0 ± 0.3 | −3.0 ± 0.3 | — | — |
| | rSIV.F/HN-CFTR | MOI 3 | 6.8 ± 0.4 | 5.7 ± 0.4 | −14.3 ± 0.4 | −13.1 ± 0.5 | 45 | 41 |
| | | MOI 10 | 12.4 ± 0.6 | 10.3 ± 0.3 | −27.5 ± 1.5 | −25.0 ± 1.4 | 87 | 79 |
| | | MOI 30 | 15.2 ± 1.3 | 13.1 ± 1.6 | −34.9 ± 2.6 | −32.8 ± 2.2 | 110 | 103 |
| | | MOI 90 | 20.9 ± 1.3 | 19.6 ± 1.1 | −37.5 ± 3.0 | −36.2 ± 2.7 | 118 | 114 |
| | Iva | — | −1.9 ± 0.1 | −1.9 ± 0.1 | −3.0 ± 0.2 | −3.0 ± 0.2 | — | — |
| | rSIV.F/HN- | MOI 3 | 11.3 ± 0.7 | 11.3 ± 0.7 | −18.9 ± 1.0 | −18.9 ± 1.0 | 60 | 60 |
| | CFTR + Iva | MOI 10 | 19.0 ± 0.9 | 19.0 ± 0.9 | −33.7 ± 2.5 | −33.7 ± 2.5 | 106 | 106 |
| | | MOI 30 | 27.7 ± 3.1 | 27.7 ± 3.1 | −44.8 ± 3.3 | −44.8 ± 3.3 | 141 | 141 |
| | | MOI 90 | 26.8 ± 3.1 | 26.8 ± 3.1 | −39.3 ± 2.5 | −39.3 ± 2.5 | 124 | 124 |
| | Luma + Iva | — | −0.1 ± 1.2 | −0.1 ± 1.2 | −0.5 ± 1.1 | −0.5 ± 1.1 | — | — |
| | Teza + Iva | — | −1 ± 0.2 | −1 ± 0.2 | −0.3 ± 0.1 | −0.3 ± 0.1 | — | — |
| | Elexa + | — | −0.9 ± 0.1 | −0.9 ± 0.1 | −1.1 ± 0.6 | −1.1 ± 0.6 | — | — |
| | Teza + Iva | | | | | | | |

LV—lentivirus,
Fsk—forskolin,
CFTR-inh172—CFTR inhibitor 172,
Iva—ivacaftor,
Luma—lumacaftor,
Teza—tezacaftor,
Elexa—elexacaftor

### Example 6—Analysis of Vector Copy Number and coCFTR Expression in Human Bronchial Epithelial Cells (HBEC, F508del/F508del, Class II) Transduced with vGM244

For experiments with clinical candidate virus vGM244 class II HBECs were transduced with both rSIV.F/HN, expressing GFP (vGM107, rSIV.F/HN-GFP) and rSIV.F/HN, expressing CFTR (vGM244, rSIV.F/HN-CFTR) with MOIs of 1, 3, 10, 30 and 90.

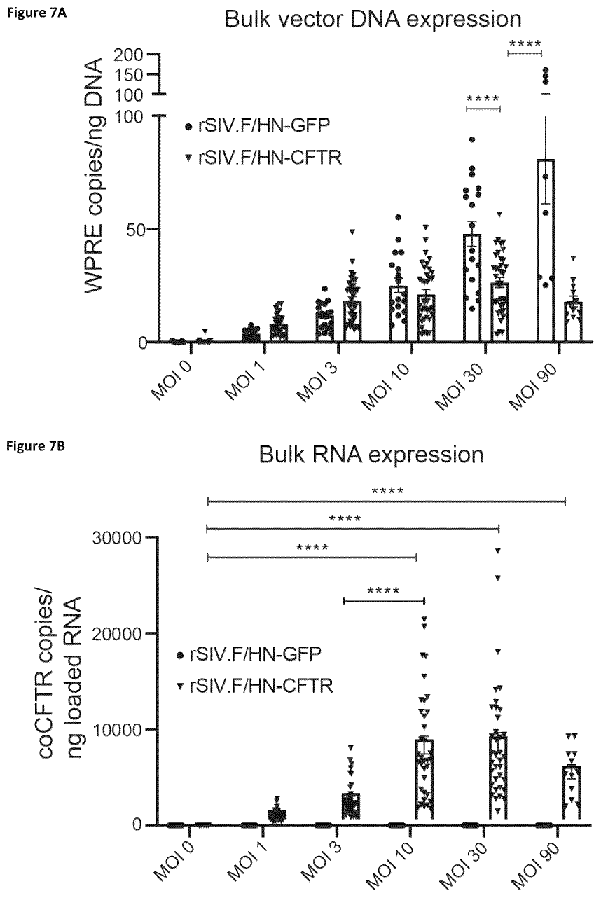

Average integrations in genome (vector copy numbers (VCN)) were analysed in DNA samples from ALI cultures and showed a dose related increase in VCN for both GFP- and CFTR-expressing rSIV.F/HN (3.8±0.6/8.3±0.8, 11.8±1.4/18.5±1.6; 25.1±3.2/21.1±2.1; 47.9±5.5/26.3±2.2; 81.0±19.8/17.9±2.4 copies/ng DNA in cells transduced at MOI 1, 3, 10, 30 and 90 with GFP/CFTR-expressing rSIV.F/HN). No difference in VCN was observed between GFP- and CFTR-expressing rSIV.F/HN at MOIs 1, 3 and 10, however at MOIs 30 and 90 the difference in VCN between GFP- and CFTR-transduced cells was significant (p<0.0001) (FIG. 7A).

Codon optimized CFTR mRNA expression was analysed in class II ALIs transduced with rSIV.F/HN-CFTR (vGM244). Dose-dependent increase in coCFTR expression was observed in cells transduced with rSIV.F/HN-CFTR in comparison to non-transduced cells, while no coCFTR expression was observed in cells transduced with rSIV.F/HN-GFP at all MOIs as expected (FIG. 7B).

### Example 7—Transduction of CF ALI Cultures (F508del/F508del, Class II) with SIV.F/HN-CFTR (vGM244) Results in Restoration of CFTR Chloride Current and Increased Ciliary Beat Frequency

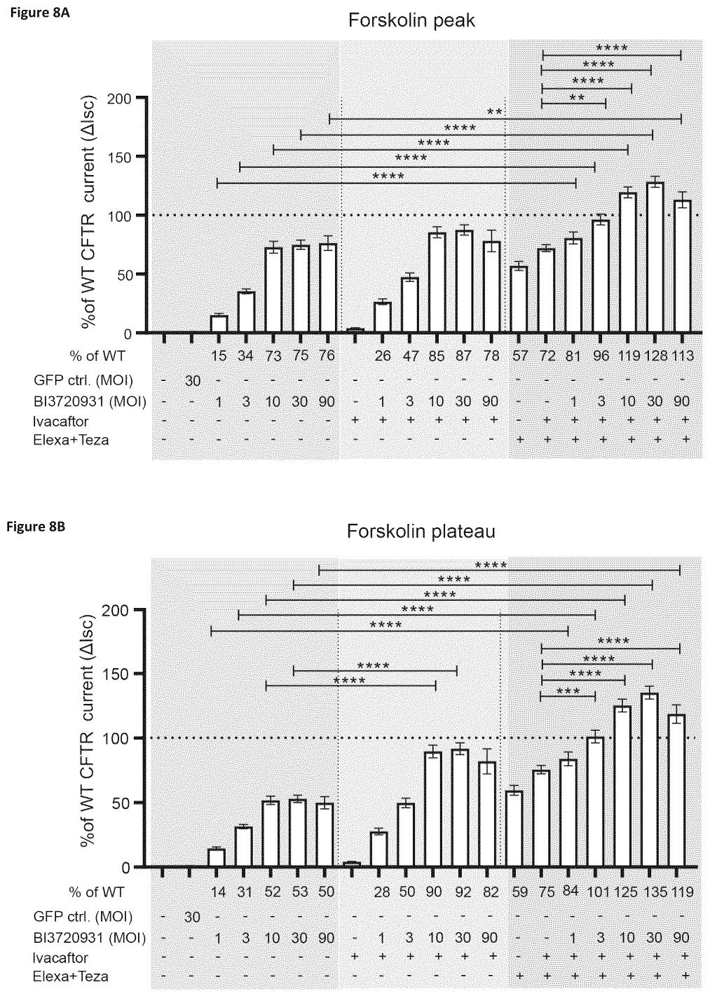

To analyse the functionality of rSIV.F/HN-CFTR-expressed channels, CFTR-mediated chloride current was measured in an Ussing chamber using rSIV.F/HN-CFTR (vGM244) transduced class II ALI cultures (Table 3). As expected, non-transduced cells (MOI 0), or cells transduced with rSIV.F/HN-GFP did not respond to forskolin or the CFTR-inhibitor confirming the absence of functional CFTR channels (FIGS. 8A and B). In contrast, a dose-related increase in CFTR chloride current was observed in ALIs transduced with rSIV.F/HN-CFTR. At an MOI of 1, there was restoration of 15±1% (peak) and 14±1% (plateau) of the non-CF chloride current. When the rSIV.F/HN-CFTR (MOI 1) was combined with the potentiator ivacaftor, there was an increase in stimulation of chloride current (26±2/28±2% for peak/plateau). At an MOI of 3, there was restoration of 34±2% (peak, p<0.0001) and 31±2% (plateau, p<0.001) of the non-CF chloride current. When the rSIV.F/HN-CFTR (MOI 3) was combined with the potentiator ivacaftor, there was an increase in stimulation of chloride current (47±3/50±4% for peak/plateau). With an increased MOI of 10, we observed higher restoration (73±5% peak, 52±3% plateau, p<0.0001) of the non-CF chloride current; a combination of MOI 10 and ivacaftor led to further increase in restoration (85±5/90±5% for peak/plateau). Higher MOIs (30 and 90) did not result in further increase in these experiments, and this was correlating with VCN and coCFTR level (FIGS. 8B and C). Thus, rSIV.F/HN-CFTR is able to completely restore the CFTR-related chloride current to non-CF values and a combination of rSIV.F/HN-CFTR with ivacaftor amplifies this effect of gene therapy by 1.2-2.0-fold. Additionally, treatment with Elexacaftor+Tezacaftor was analyzed (Trikafta without Ivacaftor) and resulted in 57±4/59±4% of restoration while treatment with Trikafta (Elexacaftor+Tezacaftor+Ivacaftor) resulted in 72±3/75±3% of restoration. Additionally, a combination of rSIV.F/HN-CFTR (vGM244) with Trikafta was analyzed and resulted in further increase in restoration effect suggesting that not only ivacaftor could give a therapeutic benefit but also ivacaftor-containing modulator therapies (FIG. 8, right-hand data set in A and B).

TABLE 3
Using chamber results for Class II F508del/F508del CF ALI cultures transduced with rSIV.F/HN (vGM244)

| | | Dose | | | ΔCFTR- | ΔCFTR- | % of | % of |
|---|---|---|---|---|---|---|---|---|
| | Treatment | of LV | ΔFskₚₑₐₖ | ΔFskₚₗₐₜₑₐᵤ | inh172ₚₑₐₖ | inh172ₚₗₐₜₑₐᵤ | WT₍ₚₑₐₖ₎ | WT₍ₚₗₐₜₑₐᵤ₎ |
| Non-CF | — | — | 31.2 ± 1.4 | 29.4 ± 1.3 | −41.8 ± 1.9 | −39.9 ± 1.7 | 100 | 100 |
| (WT) | | | | | | | | |
| CF | — | — | 0.4 ± 0.1 | 0.3 ± 0.2 | −0.2 ± 0.2 | −0.2 ± 0.1 | — | — |
| | rSIV.F/HN-GFP | MOI 90 | 0.3 ± 0.1 | 0.4 ± 0.1 | −0.3 ± 0.1 | −0.3 ± 0.1 | — | — |
| | rSIV.F/HN-CFTR | MOI 1 | 5.5 ± 0.4 | 4.9 ± 0.3 | −6.3 ± 0.6 | −5.8 ± 0.5 | 15 | 14 |
| | | MOI 3 | 10.1 ± 0.8 | 8.3 ± 0.7 | −14.3 ± 0.7 | −12.5 ± 0.6 | 34 | 31 |
| | | MOI 10 | 22.3 ± 1.8 | 12.4 ± 1.0 | −30.5 ± 2.1 | −20.6 ± 1.3 | 73 | 52 |
| | | MOI 30 | 23.0 ± 1.3 | 12.7 ± 0.7 | −31.3 ± 1.6 | −21.1 ± 1.1 | 75 | 53 |
| | | MOI 90 | 25.1 ± 1.7 | 13.0 ± 0.8 | −31.9 ± 2.6 | −19.9 ± 1.9 | 76 | 50 |
| | Iva | — | 1.0 ± 0.3 | 1.0 ± 0.3 | −1.6 ± 0.1 | −1.6 ± 0.1 | — | — |
| | rSIV.F/HN-CFTR + | MOI 1 | 10.7 ± 1.0 | 10.7 ± 1.0 | −11.1 ± 1.0 | −11.1 ± 1.0 | 26 | 28 |
| | Iva | MOI 3 | 17.3 ± 2.1 | 17.3 ± 2.1 | −19.8 ± 1.5 | −19.8 ± 1.5 | 47 | 50 |
| | | MOI 10 | 28.4 ± 2.2 | 28.4 ± 2.2 | −35.7 ± 1.9 | −35.7 ± 1.9 | 85 | 90 |
| | | MOI 30 | 30.1 ± 1.8 | 30.1 ± 1.8 | −36.6 ± 1.8 | −36.6 ± 1.8 | 87 | 92 |
| | | MOI 90 | 30.2 ± 2.8 | 30.2 ± 2.8 | −32.7 ± 3.8 | −32.7 ± 3.8 | 78 | 82 |
| | Elexa + Teza | — | 20.6 ± 0.8 | 20.5 ± 0.8 | −23.8 ± 1.6 | −23.7 ± 1.5 | 57 | 59 |
| | Elexa + Teza + | — | 26.9 ± 1.0 | 26.9 ± 1.0 | −30.1 ± 1.3 | −30.1 ± 1.3 | 72 | 75 |
| | Iva (Trikafta) | | | | | | | |
| | rSIV.F/HN-CFTR + | MOI 1 | 27.9 ± 1.7 | 27.9 ± 1.7 | −33.7 ± 2.1 | −33.4 ± 2.1 | 81 | 84 |
| | Trikafta | MOI 3 | 26.5 ± 1.4 | 26.5 ± 1.4 | −40.3 ± 1.9 | −40.3 ± 1.9 | 96 | 101 |
| | | MOI 10 | 29.3 ± 1.1 | 29.3 ± 1.1 | −50.0 ± 1.9 | −50.0 ± 2.0 | 119 | 125 |
| | | MOI 30 | 31.3 ± 1.8 | 31.3 ± 1.8 | −53.7 ± 1.9 | −53.9 ± 1.9 | 128 | 135 |
| | | MOI 90 | 32.1 ± 1.6 | 32.1 ± 1.6 | −47.3 ± 2.8 | −47.3 ± 2.8 | 113 | 119 |

LV—lentivirus,
Fsk—forskolin,
CFTR-inh172—CFTR inhibitor 172,
Iva—ivacaftor,
Luma—lumacaftor,
Teza—tezacaftor,
Elexa—elexacaftor

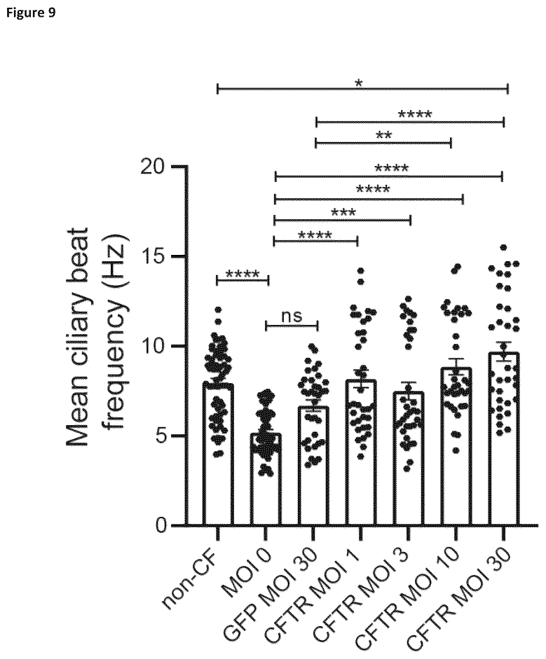

To investigate the downstream functional consequences of coCFTR expression, ciliary beat frequency (CBF) was measured as a surrogate readout of mucociliary clearance. A significant reduction in CBF in the CF ALIs (5.2±0.2 Hz in comparison to 8.0±0.2 Hz in non-CF ALIs, p<0.0001) was demonstrated. Transduction with rSIV.F/HN-CFTR was able to restore CBF to non-CF values (8.2±0.5, 7.5±0.5, 8.8±0.4 and 9.7±0.5 Hz for MOI 1, 3, 10 and 30 respectively, p<0.001, p<0.0001) (FIG. 9).

### Example 8— Analysis of Vector Copy Number and coCFTR Expression in CFTR KO Cells (Class I) Transduced with vGM244

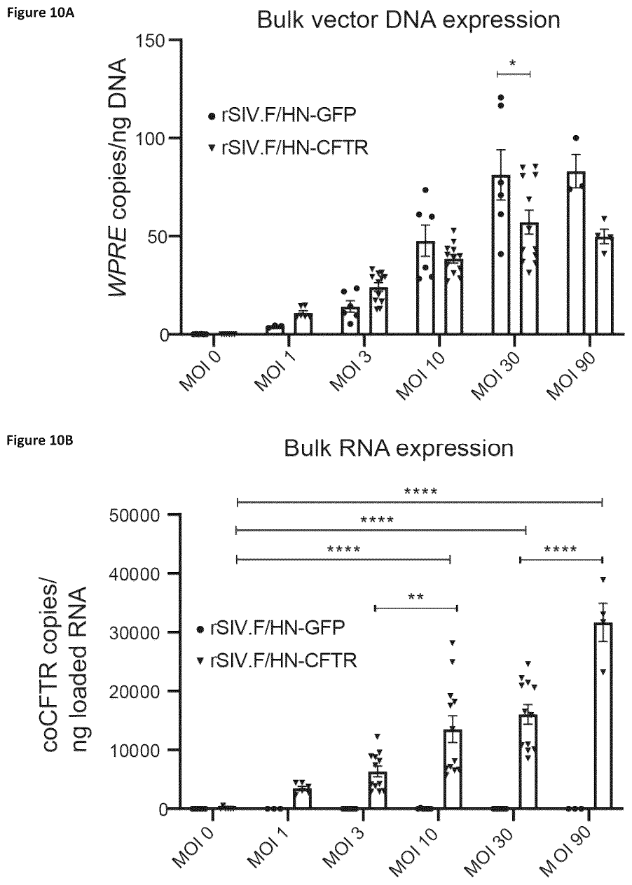

For experiments with clinical candidate virus vGM244 class I cells were transduced with both rSIV.F/HN, expressing GFP (vGM107, rSIV.F/HN-GFP) and rSIV.F/HN, expressing CFTR (vGM244, rSIV.F/HN-CFTR) with MOIs of 1, 3, 10, 30 and 90. Average integrations in genome (vector copy numbers (VCN)) were analysed in DNA samples from ALI cultures and showed a dose related increase in VCN for both GFP- and CFTR-expressing rSIV.F/HN (4.0±0.4/10.9±1.1, 14.2±2.9/24.1±2.2; 47.7±7.9/38.5±2.2; 81.3±12.8/57.2±6.2; 83.2±8.5/49.9±3.6 copies/ng DNA in cells transduced at MOI 1, 3, 10, 30 and 90 with GFP/CFTR-expressing rSIV.F/HN). No difference in VCN was observed between GFP- and CFTR-expressing rSIV.F/HN at MOIs 1, 3, 10 and 90, however at MOIs 30 the difference in VCN between GFP- and CFTR-transduced cells was significant (p<0.05) (FIG. 10A). Codon optimized CFTR mRNA expression was analysed in class I CFTR KO ALIs transduced with rSIV.F/HN-CFTR (vGM244). Dose-dependent increase in coCFTR expression was observed in cells transduced with rSIV.F/HN-CFTR in comparison to non-transduced cells, while no coCFTR expression was observed in cells transduced with rSIV.F/HN-GFP at all MOIs as expected (FIG. 10B).

### Example 9—Transduction of CFTR KO ALI Cultures (Class I) with SIV.F/HN-CFTR (vGM244) Results in Restoration of CFTR Chloride

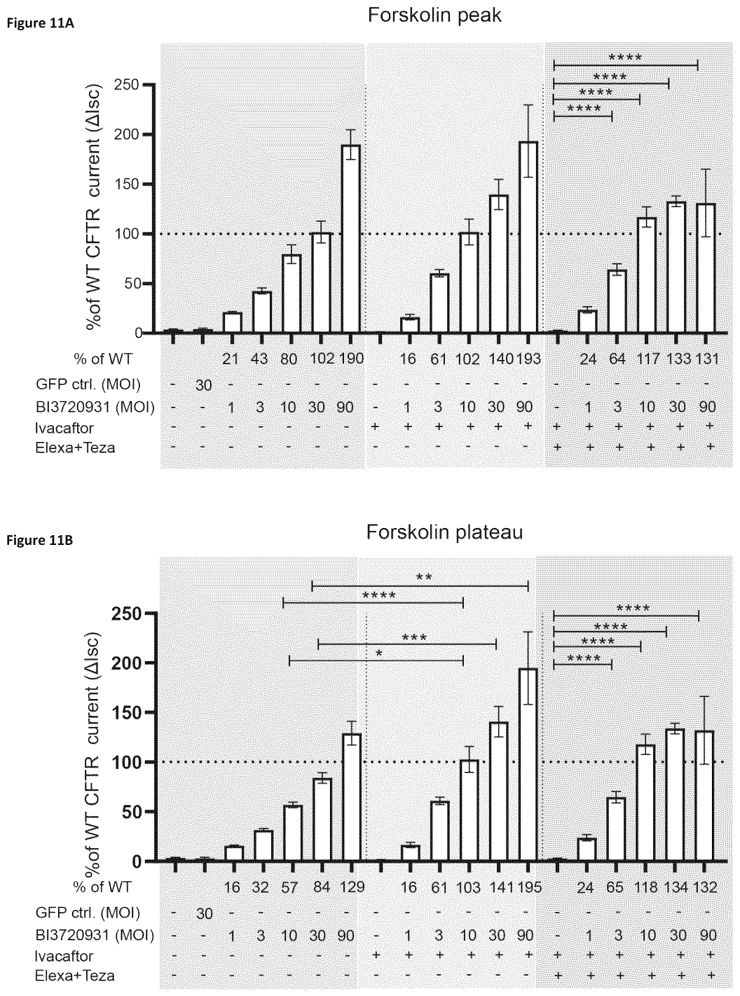

Functional analysis via Ussing chamber measurements showed a dose-related increase in CFTR current in CFTR KO ALIs (class I) transduced with rSIV.F/HN-CFTR (vGM244) (FIGS. 11A and B, Table 4). As expected, non-transduced cells (MOI 0), or cells transduced with rSIV.F/HN-GFP did not respond to forskolin or the CFTR-inhibitor confirming the absence of functional CFTR channels. In contrast, a dose-related increase in CFTR chloride current was observed in ALIs transduced with rSIV.F/HN-CFTR. At an MOI of 1, there was restoration of 21±1% (peak) and 16±1% (plateau) of the non-CF chloride current. At an MOI of 3, there was restoration of 43±3% (peak) and 32±1% (plateau) of the non-CF chloride current. When the rSIV.F/HN-CFTR (MOI 3) was combined with the potentiator ivacaftor, there was an increase in stimulation of chloride current (61±4% for both peak and plateau). With an increased MOI of 10, we observed higher restoration (80±9% peak, 57±3% plateau, p<0.001, p<0.05) of the non-CF chloride current; a combination of MOI 10 and ivacaftor led to further increase in restoration (102±11/103±13% for peak/plateau). MOI 30 resulted in further increase restoration effect (102±13/84±5% for peak/plateau, p<0.0001). MOI 30 in combination with ivacaftor resulted in 140±15/141±15% of restoration for peak/plateau (p<0.0001). Finally, MOI 90 resulted in further increase in restoration effect (190±15/129±12% for peak/plateau, p<0.0001). MOI 90 in combination with ivacaftor resulted in 193±36/195±37% of restoration for peak/plateau (p<0.0001).

Thus, rSIV.F/HN-CFTR is able to completely restore the CFTR-related chloride current to non-CF values and a combination of rSIV.F/HN-CFTR with ivacaftor amplifies this effect of gene therapy by 1.2-2.0-fold. Additionally, treatment with Trikafta (Elexacaftor+Tezacaftor+Ivacaftor) was also analysed and did not give any restoration effect for Class I cells as expected. Apart from that a combination of rSIV.F/HN-CFTR (vGM244) with Trikafta was analysed and showed restoration effect similar to the combination of rSIV.F/HN-CFTR (vGM244) with Ivacaftor. Thus, again suggesting that not only ivacaftor could give a therapeutic benefit but also ivacaftor-containing modulator therapies (FIG. 8, right-hand data set).

### Discussion

These examples provide an in-depth functional characterization of rSIV.F/HN to further prepare rSIV.F/HN vector for pre-clinical and clinical development. Specifically, these examples provide evidence for the first time in human bronchial tissues that the vector is capable of completely correcting the CF chloride defect.

TABLE 4
Ussing chamber results for Class I CFTR KO ALI cultures transduced with rSIV.F/HN (vGM244)

| | | Dose | | | ΔCFTR- | ΔCFTR- | % of | % of |
|---|---|---|---|---|---|---|---|---|
| | Treatment | of LV | ΔFskₚₑₐₖ | ΔFskₚₗₐₜₑₐᵤ | inh172ₚₑₐₖ | inh172ₚₗₐₜₑₐᵤ | WT₍ₚₑₐₖ₎ | WT₍ₚₗₐₜₑₐᵤ₎ |
| Non-CF | — | — | 26.2 ± 1.4 | 26.0 ± 1.4 | −24.5 ± 1.4 | −24.3 ± 1.5 | 100 | 100 |
| (WT) | | | | | | | | |
| CF | — | — | −0.3 ± 0.1 | −0.4 ± 0.1 | −0.9 ± 0.1 | −0.8 ± 0.1 | — | — |
| | rSIV.F/HN-GFP | MOI 90 | 0.2 ± 0.2 | −0.1 ± 0.3 | −1.0 ± 0.2 | −0.7 ± 0.2 | — | — |
| | rSIV.F/HN-CFTR | MOI 1 | 3.7 ± 0.3 | 2.3 ± 0.5 | −5.2 ± 0.2 | −3.8 ± 0.1 | 21 | 16 |
| | | MOI 3 | 9.1 ± 0.5 | 6.3 ± 0.6 | −10.4 ± 0.7 | −7.7 ± 0.3 | 43 | 32 |
| | | MOI 10 | 16.5 ± 1.5 | 10.9 ± 1.0 | −19.5 ± 2.3 | −13.8 ± 0.7 | 80 | 57 |
| | | MOI 30 | 22.0 ± 1.5 | 16.0 ± 1.6 | −24.9 ± 2.7 | −21.3 ± 1.2 | 102 | 84 |
| | | MOI 90 | 36.4 ± 3.1 | 17.6 ± 1.9 | −46.4 ± 3.6 | −31.4 ± 2.9 | 190 | 129 |
| | Iva | — | −0.7 ± 0.2 | −0.7 ± 0.2 | −0.2 ± 0.2 | −0.2 ± 0.2 | — | — |
| | rSIV.F/HN-CFTR + | MOI 1 | 3.5 ± 0.6 | 3.5 ± 0.6 | −4.0 ± 0.6 | −4.0 ± 0.6 | 16 | 16 |
| | Iva | MOI 3 | 13.9 ± 0.9 | 13.9 ± 0.9 | −14.8 ± 0.9 | −14.8 ± 0.9 | 61 | 61 |
| | | MOI 10 | 27.3 ± 2.3 | 27.3 ± 2.3 | −25.0 ± 3.2 | −25.0 ± 3.2 | 102 | 103 |
| | | MOI 30 | 44.0 ± 3.6 | 44.0 ± 3.6 | −34.2 ± 3.7 | −34.2 ± 3.7 | 140 | 141 |
| | | MOI 90 | 52.2 ± 10.1 | 52.2 ± 10.1 | −47.3 ± 8.9 | −47.3 ± 8.9 | 193 | 195 |
| | Elexa + Teza + | — | −1.0 ± 0.2 | −1.0 ± 0.2 | −0.7 ± 0.1 | −0.7 ± 0.1 | — | — |
| | Iva (Trikafta) | | | | | | | |
| | rSIV.F/HN-CFTR + | MOI 1 | 2.1 ± 0.4 | 10.2 ± 2.7 | −5.8 ± 0.8 | −5.8 ± 0.8 | 24 | 24 |
| | Trikafta | MOI 3 | 11.3 ± 1.0 | 11.3 ± 1.0 | −15.7 ± 1.4 | −15.7 ± 1.4 | 64 | 65 |
| | | MOI 10 | 28.5 ± 2.8 | 28.5 ± 2.8 | −28.7 ± 2.5 | −28.7 ± 2.5 | 117 | 118 |
| | | MOI 30 | 33.6 ± 2.6 | 33.6 ± 2.6 | −32.5 ± 1.3 | −32.5 ± 1.3 | 133 | 134 |
| | | MOI 90 | 24.0 ± 6.9 | 24.0 ± 6.9 | −32.1 ± 8.3 | −32.1 ± 8.3 | 131 | 132 |

LV—lentivirus,
Fsk—forskolin,
CFTR-inh172—CFTR inhibitor 172,
Iva—ivacaftor,
Luma—lumacaftor,
Teza—tezacaftor,
Elexa—elexacaftor

Remarkably, these experiments demonstrate that CFTR modulators, particularly CFTR potentiators, achieve a greater than expected potentiation of the CFTR transgene expressed by rSIV.F/HN. In particular, the effect of the CFTR modulator, particularly the CFTR potentiator, and rSIV.F/HN-CFTR combination is greater than the additive effects of the separate effects of the CFTR modulator, particularly the CFTR potentiator, and rSIV.F/HN-mediated CFTR expression.

A relationship between transduced cell number and degree of correction has been established, and the vector integration profile of such a self-inactivating lentiviral vector in a relevant cell type for lung gene therapy assessed. These data provide further support for the translation of this vector into first-in-man trials.

Previous studies have suggested that a range of 5-25% corrected cells should be sufficient to restore the CFTR chloride current to normal values. This is also supported by previous study that show that CF individuals with certain “mild” mutations that retain 10% of normal CFTR expression per cell do generally not suffer from disease. Therefore, without being bound by the theory, the present work indicates that even a low percentage of transduced cells, combined with a strong promotor driving CFTR expression is sufficient to provide significant restoration of the CFTR related chloride current.

As exemplified herein for the first time, the effect of gene therapy can be further enhanced with the clinically approved CFTR potentiator ivacaftor and ivacaftor containing products like TRIKAFTA.

Ivacaftor and other CFTR potentiators, as well as products containing ivacaftor (e.g. TRIKAFTA) or other potentiators, act by increasing channel open probability. Given the typical open probability of wild-type CFTR chloride channels of approximately 0.4, this effect is potentially clinically important. The combination of gene therapy plus the potentiator reduced the number of transduced cells needed to achieve full restoration of the chloride current and decreased the needed MOI by approximately 1.2-2.0 fold. This has significant potential therapeutic and economic importance, and surprising given the greater than expected potentiation of the CFTR transgene expressed by rSIV.F/HN. There are potentially benefits in terms of efficacy, cost-of-goods and applicability to a broad swathe of the CF population given that ivacaftor is a constituent of currently used modulators.

Whilst the optimal airway cell type to target for successful CF gene therapy is unclear, recent studies have provided indications. Utilizing single-cell RNA-seq technologies it has been shown that apart from the ionocytes, which is a rare cell type, CFTR is mainly expressed by SCGB1A1+ club cells and to a lesser extent by basal cells, collectively accounting for ^{˜}80% of all CFTR+ cells. The rSIV.F/HN vector is able to transduce all these relevant types of cells in the murine lung in vivo, and here further confirmation for sufficient transduction efficiency in human bronchial epithelial cells has been obtained.

Apart from providing a novel and sustainable treatment option for a broad range of CFTR patients, including patients affected by the most common F508del/F508del mutation, gene therapy is especially attractive for those carrying Class I mutations, which result in complete absence of CFTR protein and for whom there is currently no modulator treatment available. In order to analyse the functional effects of rSIV.F/HN in Class I mutation cells, a novel CFTR knock-out cell line was generated. Transduction of these cells with rSIV.F/HN again resulted in robust transgene expression in all relevant cell types and resulted in restoration of forskolin-stimulated CFTR current to wild-type levels. As expected, none of the modulators produced an effect on the CFTR chloride current. In contrast rSIV.F/HN was able to fully restore CFTR function in a dose-dependent manner, as was the case for the F508del/F508del cells. Further, similarly to Class II mutation cells, we observed an unexpected increase in effect with ivacaftor and TRIKAFTA. These data suggest that rSIV.F/H N is a viable contender for the treatment of patients carrying null mutations, or those patients who are insensitive to, or unable to tolerate modulators. Where such patients can tolerate modulators, there is the further opportunity to improve or optimise treatment.

The downstream consequences of chloride current restoration through assessment of Ciliary beat frequency (CBF) as a surrogate readout for mucociliary clearance (MCC) was also assessed. Clinical data suggest that MMC becomes impaired with increasing disease severity, likely related to lack of adequate hydration of both the mucus and the underlying airway surface liquid. The CF ALIs used in this study demonstrated a reduced ciliary beat frequency (CBF), likely secondary to these two parameters and rSIV.F/HN CFTR restored CBF in F508del/F508del ALIs to wildtype levels. These findings provide a further indication that rSIV.F/HN CFTR could improve lung physiology in CF patients in vivo, through an effect on MCC.

In conclusion, this work demonstrates the high transduction efficiency and consequent complete functional correction of the CF chloride defect in human ALIs, both in primary HBE cells from F508del/F508del patients as well as in a novel CFTR KO hSABCi cell line as a model of Class I homozygous null mutations. The potentiator ivacaftor showed a surprising and greater than expected effect when combined with rSIV.F/HN. These data suggest that this lentiviral vector is a leading candidate for the treatment of CF patients independent of mutation class and, that combination therapy using this vector with one or more CFTR modulators, particularly a potentiator such as ivacaftor, or a product containing a potentiator such as ivacaftor (e.g. TRIKAFTA) is an attractive prospect for treating CF, with an initial focus on modulator insensitive patients.

1. A method of treating cystic fibrosis (CF) in a subject in need thereof, comprising administering to the subject a therapeutically effective amount of each of (i) a lentiviral vector pseudotyped with hemagglutinin-neuraminidase (HN) and fusion (F) proteins from a respiratory paramyxovirus, wherein said lentiviral vector comprises a cystic fibrosis transmembrane conductance regulator (CFTR) transgene and (ii) a CFTR modulator.

2. The method of treating CF according to claim 1, wherein the lentiviral vector is a SIV vector and the respiratory paramyxovirus is a Sendai virus.

3. The method of treating CF according to claim 1, wherein the transgene is:
(i) operably linked to a promoter selected from the group consisting of a cytomegalovirus (CMV) promoter, elongation factor 1a (EF1a) promoter, and a hybrid human CMV enhancer/EF1a (hCEF) promoter; and/or
(ii) a codon-optimised CFTR transgene, which optionally comprises or consist of a nucleotide sequence having at least 90% identity to SEQ ID NO: 1.

4. The method of treating CF according to claim 1, wherein the lentiviral vector comprises a hybrid human CMV enhancer/EF1a (hCEF) promoter, which optionally comprises or consist of a nucleotide sequence having at least 90% identity to SEQ ID NO: 2.

5. (canceled)

6. The method of treating CF according to claim 1, wherein the lentiviral vector is produced using codon-optimised plasmids wherein preferably the lentiviral vector is produced using (i) pGM691 and/or (ii) pGM830 or pGM326; and most preferably also using pGM299, pGM301 and/or pGM303.

7. (canceled)

8. The method of treating CF according to claim 1, wherein the lentiviral vector is vGM058, vGM195 or vGM244.

9. The method of treating CF according to claim 1, wherein the lentiviral vector is an SIV vector pseudotyped with Sendai virus hemagglutinin-neuraminidase (HN) and fusion (F) proteins, wherein: said vector comprises a modified retroviral RNA sequence which comprises or consists of a nucleic acid sequence of SEQ ID NO: 16.

10. The method of treating CF according to claim 1, wherein the lentiviral vector:
(i) comprises an F protein with a first subunit which comprises or consists of an amino acid sequence of SEQ ID NO: 19 and a second subunit which comprises or consists of an amino acid sequence of SEQ ID NO: 20; and/or
(ii) further comprises:
(a) a p17 protein comprising or consisting of an amino acid sequence of SEQ ID NO: 22;
(b) a p24 protein comprising or consisting of an amino acid sequence of SEQ ID NO: 23;
(c) a p8 protein comprising or consisting of an amino acid sequence of SEQ ID NO: 24;
(d) a protease comprising or consisting of an amino acid sequence of SEQ ID NO: 25;
(e) a p51 protein comprising or consisting of an amino acid sequence of SEQ ID NO: 26;
(f) a p15 protein comprising or consisting of an amino acid sequence of SEQ ID NO: 27,
(g) a p31 protein comprising or consisting of an amino acid sequence of SEQ ID NO: 28,
(h) a Gag protein comprising or consisting of an amino acid sequence of SEQ ID NO: 29, and/or
(i) a Pol protein comprising or consisting of an amino acid sequence of SEQ ID NO: 30;
wherein optionally the vector comprises each of (a) to (g).

11. (canceled)

12. The method of treating CF according to claim 1, wherein the CFTR modulator is a CFTR potentiator and/or a CFTR corrector, preferably a CFTR potentiator and wherein the CFTR modulator selected from ivacaftor, tezacaftor, elexacaftor or lumacaftor, or a combination thereof, preferably ivacaftor.

13. (canceled)

14. (canceled)

15. A method of treating cystic fibrosis (CF) in a subject in need thereof, comprising administering to the subject a therapeutically effective amount of each of
(A) an SIV vector pseudotyped with Sendai virus hemagglutinin-neuraminidase (HN) and fusion (F) proteins, wherein:
(a) said vector comprises a modified retroviral RNA sequence which comprises or consists of a nucleic acid sequence of SEQ ID NO: 16; and
(b) the F protein comprises a first subunit which comprises or consists of an amino acid sequence of SEQ ID NO: 19 and a second subunit which comprises or consists of an amino acid sequence of SEQ ID NO: 20; and
(B) ivacaftor.

16. The method of treating CF according to claim 1, wherein the vector further comprises one or more of:
(a) a p17 protein comprising or consisting of an amino acid sequence of SEQ ID NO: 22;
(b) a p24 protein comprising or consisting of an amino acid sequence of SEQ ID NO: 23;
(c) p8 protein comprising or consisting of an amino acid sequence of SEQ ID NO: 24;
(d) a protease comprising or consisting of an amino acid sequence of SEQ ID NO: 25;
(e) a p51 protein comprising or consisting of an amino acid sequence of SEQ ID NO: 26;
(f) a p15 protein comprising or consisting of an amino acid sequence of SEQ ID NO: 27;
(g) a p31 protein comprising or consisting of an amino acid sequence of SEQ ID NO: 28;
(h) a Gag protein comprising or consisting of an amino acid sequence of SEQ ID NO: 29; and/or
(i) a Pol protein comprising or consisting of an amino acid sequence of SEQ ID NO: 30;
wherein optionally the vector comprises each of (a) to (g).

17. The method of treating CF according to claim 1, wherein a patient to be treated has at least one class I, class II, class III, class IV, class V or class VI CFTR mutation and wherein preferably wherein the patient to be treated has at least one class I and/or class II CFTR mutation.

18. (canceled)

19. The method of treating CF according to claim 17, wherein:
(a) the combination of a lentiviral vector and a CFTR modulator is suitable for use independent of the CFTR mutation of the patient; or
(b) the patient to be treated has:
i. at least one class I CFTR mutation selected from G542X, W1282X and/or R553C; and/or
ii. at least one class II CFTR mutation selected from F508del, N1303K and/or I507del.

20. The method of treating CF according to claim 1, wherein the lentiviral vector and the CFTR modulator are administered simultaneously or sequentially.

21. The method of treating CF according to claim 1, wherein:
(a) the lentiviral vector is administered by inhalation; and/or
(b) the CFTR modulator is administered orally.

22. The method of treating CF according to claim 1, wherein:
(a) the lentiviral vector is administered at a dose of between about 8⁸ to about 10¹⁴ transducing units (TU), preferably a dose of between about 10⁶ to about 10¹² TU, wherein optionally the lentiviral vector is administered at a frequency of every 3 months, every 6 months, every 12 months, every 24 months, every 36 months or every 48 months; and/or
(b) the CFTR modulator is administered at a concentration used for monotherapy of each modulator or lower.

23. The method of treating CF according to claim 1, wherein treatment restores CFTR activity to at least 10% of CFTR activity in a healthy control and wherein preferably the treatment restores CFTR activity to at least 50% of CFTR activity in a healthy control.

24. (canceled)

25. The method of treating CF according to claim 1, wherein treatment increases CFTR activity by at least 1.2 fold compared with treatment with the lentiviral vector alone and wherein preferably the treatment increases CFTR current by about 1.3 fold to about 3 fold or about 1.3 fold to about 1.8 fold compared with treatment with the lentiviral vector alone.

26. (canceled)

27. The method of treating CF according to claim 1, wherein the patient to be treated:
(i) has a class I CFTR mutation and the treatment: (i) restores CFTR activity to at least 10% of CFTR activity in a healthy control; and/or (ii) increases CFTR current by about 1.3 fold to about 1.8 fold or about 1.3 fold to about 3 fold compared with treatment with the lentiviral vector alone; or
(ii) has a class II CFTR mutation and the treatment: (i) restores CFTR activity to at least 10% of CFTR activity in a healthy control; and/or (ii) increases CFTR current by about 1.3 fold to about 3 fold or about 1.3 fold to about 1.8 fold compared with treatment with the lentiviral vector alone.

28. (canceled)

29. The method of treating CF according to claim 1, wherein a transduction rate of between about 10% to about 20%, preferably between about 14% to about 17% is sufficient to achieve a therapeutic effect on CFTR activity as defined in any one of claims 23 to 28.
